(19) United States
(12) Patent Application Publication
(10) Pub. No.: US 2023/0023446 A1
(43) Pub. Date: Jan. 26, 2023

(54) PROCESSES FOR PRODUCING FERMENTATION PRODUCTS

(71) Applicant: Novozymes A/S, Bagsvaerd (DK)

(72) Inventors: Chee-Leong Soong, Raleigh, NC (US); Yuma Kurakata, Chiba (JP); Brian Frederick Ohman, Raleigh, NC (US)

(73) Assignee: Novozymes A/S, Bagsvaerd (DK)

(21) Appl. No.: 17/781,973

(22) PCT Filed: Dec. 16, 2020
(86) PCT No.: PCT/US2020/065293

§ 371 Date: Jun. 2, 2022

Related U.S. Application Data
(60) Provisional application No. 62/948,415, filed on Dec. 16, 2019.

(51) Int. Cl. C12N 9/24; C12P 19/00; C12P 7/06

(52) U.S. Cl. CPC C12N 9/248; C12P 19/00; C12P 7/06; C12Y 302/01008

(57) ABSTRACT

The present invention relates to processes for producing fermentation products from starch-containing material, wherein a thermostable xylanase that is resistance to inhibition by metal ions in the liquefying starch-containing material is present and/or added during liquefaction.

### FIELD OF THE INVENTION

The present invention relates to processes for producing fermentation products, especially ethanol, from starch-containing material, wherein a starch-containing material is liquefied in the presence of a xylanase that is resistant to inhibition by metal ions present in the liquefying starch-containing material.

### REFERENCE TO A SEQUENCE LISTING

This application contains a Sequence Listing in computer readable form. The computer readable form is incorporated herein by reference.

### BACKGROUND OF THE INVENTION

Production of fermentation products, such as ethanol, from starch-containing material is well-known in the art. Industrially two different kinds of processes are used today. The most commonly used process, often referred to as a “conventional process”, includes liquefying gelatinized starch at high temperature using typically a bacterial alpha-amylase, followed by simultaneous saccharification and fermentation carried out in the presence of a glucoamylase and a fermentation organism. Another well-known process, often referred to as a “raw starch hydrolysis”—process (RSH process), includes simultaneously saccharifying and fermenting granular starch below the initial gelatinization temperature typically in the presence of at least a glucoamylase.

Despite significant improvement of fermentation product production processes over the past decade a significant amount of residual starch material is not converted into the desired fermentation product, such as ethanol.

Therefore, there is still a desire and need for providing processes for producing fermentation products, such as ethanol, from starch-containing material that can provide a higher fermentation product yield, or other advantages, compared to conventional processes.

### SUMMARY OF THE INVENTION

The present invention relates to processes of producing fermentation products, such as especially ethanol, from starch-containing material using a fermenting organism, wherein a starch-containing material is liquefied in the presence of a xylanase that is resistant to inhibition by metal ions present in the liquefying starch-containing material. The invention also relates to compositions for use in processes of the invention. The metal-ion inhibition resistant xylanases of the present invention exhibit improved activity in hydrolyzing corn fiber and liberate more fiber-bound starch compared to non-metal ion inhibition resistant xylanases. The metal-ion inhibition resistant xylanases of the present invention also generate greater amounts of short-chain oligosaccharides upon completion of liquefaction, such as DP1 to DP6 oligosaccharides, compared to non-metal ion inhibition resistant xylanases.

In the first aspect the invention relates to processes for producing fermentation products, such as preferably ethanol, from starch-containing material comprising the steps of:

i) liquefying a starch-containing material at a temperature above the initial gelatinization temperature, preferably between 80-90° C., using:
an alpha-amylase, such as a bacterial alpha-amylase;
a xylanase that is resistant to inhibition by metal ions in the liquefying starch-containing material, and further having a Melting Point (DSC) above 80° C.;
optionally an endoglucanase having Melting Point (DSC) above 70° C.;
   ii) saccharifying using a carbohydrate-source generating enzyme;
   iii) fermenting using a fermenting organism.

In a preferred embodiment the xylanase, especially a xylanase from the genus Thermotoga, preferably has a Melting Point (DSC) above 82° C., such as above 84° C., such as above 86° C., such as above 88° C., such as above 88° C., such as above 90° C., such as above 92° C., such as above 94° C., such as above 96° C., such as above 98° C., such as above 100° C., such as between 80° C. and 110° C., such as between 82° C. and 110° C., such as between 84° C. and 110° C.

Examples of suitable thermostable xylanases, in particular xylanases from the genus Thermotoga, include the xylanase shown in SEQ ID NOs: 2 herein, e.g., derived from a strain of Thermotoga maritima; the xylanase shown in SEQ ID NO: 3 herein, e.g., derived from a strain of Thermotoga neapolitana; the xylanase shown in SEQ ID NO: 4 herein, e.g., derived from a strain of Thermotoga naphthophila; or polypeptides having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity, such as 100% identity to the mature part of any of the polypeptides of SEQ ID NOs: 2, 3, and 4 herein, respectively.

In another aspect the invention relates to compositions comprising: —an alpha-amylase; —a metal ion inhibition resistant xylanase having a Melting Point (DSC) above 80° C., preferably above 85° C., especially above 90° C., in particular above 95° C.; —optionally an endoglucanase; —optionally a protease; —optionally a carbohydrate-source generating enzyme.

Other enzymes such as pullulanases and phytases may also be comprised in the composition of the invention.

In a preferred embodiment, the composition of the invention comprises—an alpha-amylase, preferably a bacterial alpha-amylase; —a metal ion inhibition resistant xylanase having a Melting Point (DSC) above 80° C., preferably above 85° C., especially above 90° C., in particular above 95° C.; —protease having Melting Point (DSC) above 80° C., preferably above 85° C., especially above 90° C., in particular above 95° C.

In a preferred embodiment, the composition of the invention comprises—an alpha-amylase, preferably a thermostable bacterial alpha-amylase; —a thermostable metal ion inhibition resistant xylanase having a Melting Point (DSC) above 80° C., preferably above 85° C., especially above 90° C., in particular above 95° C.; and—a thermostable protease having Melting Point (DSC) above 80° C., preferably above 85° C., especially above 90° C., in particular above 95° C.

### BRIEF DESCRIPTION OF THE DRAWINGS

FIG. 1 is a graph showing the temperature optimum of an exemplary metal ion-inhibition resistant xylanase of the present invention, for example, the Tm xylanase (SEQ ID NO: 2).

FIG. 2 is a graph showing the temperature stability of an exemplary metal ion-inhibition resistant xylanase of the present invention, for example, the Tm xylanase (SEQ ID NO: 2).

### DETAILED DESCRIPTION OF THE INVENTION

The inventors have found that certain thermostable xylanases, such as the Dictyoglomus thermophilum xylanase of SEQ ID NO: 1 are significantly inhibited by metal ions, in contrast to certain thermostable xylanases, such as xylanases from the genus Thermotoga, for instance the Thermotoga maritima xylanase of SEQ ID NO: 2, which exhibit resistance to inhibition by metal ions. Surprisingly, the inventors unexpectedly found that whereas the relative activity of the Dictyoglomus thermophilum xylanase of SEQ ID NO: 1 decreased by between 40% to 80% in the presence of the average concentrations of iron (Fe), Zinc (Zn) and Copper (Cu) ions typically found in liquefied corn mash, xylanases from the genus Thermotoga, for instance the Thermotoga maritima xylanase of SEQ ID NO: 2, retained 86% of its relative activity in the presence of the average concentration of copper ions in liquified corn mash, retained 73% of its relative activity in the presence of the average concentration of iron ions in liquefied corn mash, and retained 98% of its relative activity in the presence of the average concentration of zinc ions in liquefied corn mash, demonstrating a marked resistance to metal ion inhibition (See Example 6).

The inventors also unexpectedly found that the metal-ion inhibition resistant thermostable xylanases described herein, such as xylanases from the genus Thermotoga, for instance Thermotoga maritima xylanase of SEQ ID NO: 2, exhibited improved activity in hydrolyzing corn fiber and liberated more fiber-bound starch compared to non-metal ion inhibition resistant xylanases, such as the Dictyoglomus thermophilum xylanase of SEQ ID NO: 1.

In addition, the inventors surprisingly found that the metal-ion inhibition resistant thermostable xylanases described herein, such as xylanases from the genus Thermotoga, for instance Thermotoga maritima xylanase of SEQ ID NO: 2, generated greater amounts of short-chain oligosaccharides upon completion of liquefaction, such as DP1 to DP6 oligosaccharides, compared to non-metal ion inhibition resistant xylanases.

The xylanases of the invention from Thermotoga were observed to contain the motif comprised of the amino acids tyrosine (Y), isoleucine (I), threonine (T), glutamic acid (E), methionine (M), and aspartic acid (D), wherein the glutamic acid residue is a catalytic residue. The present invention contemplates using any Thermotoga xylanase, for instance from the GH10 family, that contains the YITEMD (SEQ ID NO: 30) motif in a composition or process of the invention.

In the first aspect the invention relates to processes for producing fermentation products, such as preferably ethanol, from starch-containing material comprising the steps of: i) liquefying a starch-containing material at a temperature above the initial gelatinization temperature in the presence of a thermostable xylanase that is resistant to inhibition by metal ions in the liquefying starch-containing material; ii) saccharifying using a carbohydrate-source generating enzyme; and iii) fermenting using a fermenting organism. In an embodiment, liquefying step i) is performed in the presence of an alpha-amylase, i.e., a thermostable alpha-amylase, preferably a thermostable bacterial alpha-amylase and/or a thermostable protease.

In an embodiment of the first aspect, liquefying the starch-containing material in step i) using the thermostable xylanase that is resistant to inhibition by metal ions in the liquefying starch-containing material decreases the amount of residual starch present at the end of liquefying step i) compared to the amount of residual starch present at the end of liquefying step i) when using a non metal-ion inhibition resistant thermostable xylanase in liquefying step i).

In an embodiment of the first aspect, liquefying the starch-containing material in step i) using the thermostable xylanase that is resistant to inhibition by metal ions in the liquefying starch-containing material increases the amount of short chain oligosaccharides at the end of liquefying step i) compared to the amount of short chain oligosaccharides at the end of liquefying step i) when using a non metal-ion inhibition resistant thermostable xylanase in liquefying step i).

In the second aspect the invention relates to a process for decreasing the amount of residual starch present at the end of liquefaction in a process for producing a fermentation product from a starch-containing material, comprising: i) liquefying a starch-containing material with a thermostable xylanase that is resistant to inhibition by metal ions in the liquefying starch-containing material to produce a liquefact, wherein the liquefact has a decreased amount of residual starch compared to a liquefact produced using a xylanase that is not resistant to inhibition due to metal ions present in the liquefying starch-containing material. In an embodiment of the second aspect, the liquefact is subjected to the steps of ii) saccharifying using a carbohydrate-source generating enzyme; and iii) fermenting using a fermenting organism.

As used herein, “thermostable” means that the polypeptide having a particular enzymatic activity (i.e., enzyme, e.g., xylanase, alpha-amylase, protease) retains a significant amount of its activity (e.g., specific activity, relative activity, etc.) within a relevant temperature range over for a particular period of time needed for the enzyme to carry out its function. For example, the presently disclosed enzymes used in liquefying step i) exhibit thermostability at temperatures ranging from about 75 C to 100 C, preferably from about 80 to 95, more preferably from about 82 to 92 C, or about 85 C, about 88 C or about 90 C. The term “thermostable” also encompasses enzymes that have a temperature optimum within the relevant temperature range, though the present disclosure contemplates enzymes with higher or lower temperature optima as long as the enzyme retains the significant amount of its activity within the relevant temperature range over the relevant time period (e.g., enzymes used in liquefaction would retain their activity from any where from 10 minutes to 2 hours at temperature ranging from 75 C to 100 C). The thermostable enzymes of the present invention (e.g., metal-ion inhibition resistant thermsostable xylanase, thermsostable protease, thermsostable alpha-amylase, thermsostable endoglucanase, thermsostable glucoamylase, thermsostable pullulanase, phytase, etc.) retain at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of their activity when used in liquefaction for up to 10 minutes, 15 minutes, 20 minutes, 25 minutes, 30 minutes, 45 minutes, 1 hour, 1.2 hours, 1.4 hours 1.5 hours, 1.6 hours, 1.8 hours, or up to 2 hours at temperatures of at least 75 C, 80 C, 82 C, 84 C, 85 C, 86 C, 88 C, 90 C, 92 C, 94 C, 95 C, 96 C, 98 C or up to 100 C. The thermostable enzymes of the present invention (e.g., metal-ion inhibition resistant thermsostable xylanase, thermsostable protease, thermsostable alpha-amylase, thermsostable endoglucanase, thermsostable glucoamylase, thermsostable pullulanase, phytase, etc.) preferably have a Melting Point (DSC) from about 75 C to about 110 C, for example above about 80 C, about 82 C, about 84 C, about 85 C, about 86 C, about 88 C, about 90 C, about 92 C, about 94 C, about 95 C, about 96 C, about 98 C, about 100 C, about 105 C, or up to about 110 C. The DSC melting point may be determined by techniques available to the skilled artisan, for example by the Differential Scanning Calotimetry assay described for Xylanases below. In addition, activity assays can be used to determine the activity of the enzymes at different temperatures to determine the temperature optimum of a particular thermostable enzyme of the present invention, as well as to determine how much activity the thermostable enzyme retains within a particular temperature range or over a period of time. Examples of activity assays are described in the examples below.

Those skilled in the art will appreciate that the extent to which residual starch is decreased in the liquefact may vary, for instance, depending on the concentration of the xylanase, amongst other factors which would be apparent to the skilled artisan. For instance, the residual starch may decrease by at least 1%, at least 1.5%, at least 2%, at least 2.5%, at least 3%, at least 3.5%, at least 4%, at least 4.5%, at least 5%, at least 6%, at least 7%, at least 8%, at least 9%, at least 10%, at least 15%, at least 20%, at least 25%, at least 30%, at least 35%, at least 40%, or at least 50% compared to the amount of residual starch in the liquefact in the absence of thermostable metal ion inhibition resistant xylanase. The assay described in Example 7 can be used to determine the amount of residual starch present in a liquefact when liquefying a starch-containing material using a metal-ion inhibition resistant xylanase and comparisons to controls (e.g., using a non-metal ion inhibition resistant xylanase, such as the xylanase of SEQ ID NO: 1) to determine the extent to which residual starch is decreased using the metal ion inhibition resistant xylanases of the present invention.

In the third aspect the invention relates to a process for increasing the amount of short-chain oligosaccharides present at the end of liquefaction in a process for producing a fermentation product from a starch-containing material, comprising: i) liquefying a starch-containing material with thermostable xylanase that is resistant to inhibition by metal ions in the liquefying starch-containing material to produce a liquefact, wherein the liquefact has an increased amount of short-chain oligosaccharides compared to a liquefact produced using a xylanase that is not resistant to inhibition due to metal ions present in the liquefying starch-containing material. In an embodiment of the third aspect, the liquefact is subjected to the steps of ii) saccharifying using a carbohydrate-source generating enzyme; and iii) fermenting using a fermenting organism.

As used herein, the phrase “short-chain oligosaccharides” refers to oligosaccharides from DP1 to DP6, including for instance glucose (DP1), maltose (DP2), maltotriose (DP3) maltotetraose (DP4), maltopentaose (DP5), and maltohexaose (DP6). Those skilled in the art will appreciate that the extent to which short-chain oligosaccharides are increased in the liquefact may vary, for instance, depending on the concentration of the xylanase, amongst other factors which would be apparent to the skilled artisan. For instance, the overall amount of short-chain oligosaccharides may increase by at least 1%, at least 1.5%, at least 2%, at least 2.5%, at least 3%, at least 3.5%, at least 4%, at least 4.5%, at least 5%, at least 6%, at least 7%, at least 8%, at least 9%, at least 10%, at least 15%, at least 20%, at least 25%, at least 30%, at least 35%, at least 40%, or at least 50% compared to the amount of residual starch in the liquefact in the absence of thermostable metal ion inhibition resistant xylanase. The assay described in Example 8 can be used to determine the amount of short-chain oligosaccharides present in a liquefact when liquefying starch-containing material using a metal-ion inhibition resistant xylanase and comparisons to controls (e.g., using a non-metal ion inhibition resistant xylanase, such as the xylanase of SEQ ID NO: 1) to determine the extent to which short-chain oligosacchardies are increased using the metal ion inhibition resistant xylanases of the present invention.

In an embodiment, the amount of DP1 present in the liquefact is increased by at least 1%, at least 1.5%, at least 2%, at least 2.5%, at least 3%, at least 3.5%, at least 4%, at least 4.5%, at least 5%, at least 6%, at least 7%, at least 8%, at least 9%, at least 10%, at least 15%, at least 20%, at least 25%, at least 30%, at least 35%, at least 40%, or at least 50% compared to the amount of DP1 in the liquefact in the absence of a thermostable metal ion inhibition resistant xylanase of the present invention. In an embodiment, the amount of DP2 present in the liquefact is increased by at least 1%, at least 1.5%, at least 2%, at least 2.5%, at least 3%, at least 3.5%, at least 4%, at least 4.5%, at least 5%, at least 6%, at least 7%, at least 8%, at least 9%, at least 10%, at least 15%, at least 20%, at least 25%, at least 30%, at least 35%, at least 40%, or at least 50% compared to the amount of DP2 in the liquefact in the absence of a thermostable metal ion inhibition resistant xylanase of the present invention. In an embodiment, the amount of DP3 present in the liquefact is increased by at least 1%, at least 1.5%, at least 2%, at least 2.5%, at least 3%, at least 3.5%, at least 4%, at least 4.5%, at least 5%, at least 6%, at least 7%, at least 8%, at least 9%, at least 10%, at least 15%, at least 20%, at least 25%, at least 30%, at least 35%, at least 40%, or at least 50% compared to the amount of DP3 in the liquefact in the absence of a thermostable metal ion inhibition resistant xylanase of the present invention. In an embodiment, the amount of DP4 present in the liquefact is increased by at least 1%, at least 1.5%, at least 2%, at least 2.5%, at least 3%, at least 3.5%, at least 4%, at least 4.5%, at least 5%, at least 6%, at least 7%, at least 8%, at least 9%, at least 10%, at least 15%, at least 20%, at least 25%, at least 30%, at least 35%, at least 40%, or at least 50% compared to the amount of DP4 in the liquefact in the absence of a thermostable metal ion inhibition resistant xylanase of the present invention. In an embodiment, the amount of DP5 present in the liquefact is increased by at least 1%, at least 1.5%, at least 2%, at least 2.5%, at least 3%, at least 3.5%, at least 4%, at least 4.5%, at least 5%, at least 6%, at least 7%, at least 8%, at least 9%, at least 10%, at least 15%, at least 20%, at least 25%, at least 30%, at least 35%, at least 40%, or at least 50% compared to the amount of DP5 in the liquefact in the absence of a thermostable metal ion inhibition resistant xylanase of the present invention. In an embodiment, the amount of DP6 present in the liquefact is increased by at least 1%, at least 1.5%, at least 2%, at least 2.5%, at least 3%, at least 3.5%, at least 4%, at least 4.5%, at least 5%, at least 6%, at least 7%, at least 8%, at least 9%, at least 10%, at least 15%, at least 20%, at least 25%, at least 30%, at least 35%, at least 40%, or at least 50% compared to the amount of DP6 in the liquefact in the absence of a thermostable metal ion inhibition resistant xylanase of the present invention.

Steps ii) and iii) may be carried out either sequentially or simultaneously. In a preferred embodiment steps ii) and iii) are carried out simultaneously. The xylanase, preferably having a Melting Point (DSC) above 80° C.; an alpha-amylase, preferably a thermostable bacterial alpha-amylase, and optionally a thermostable endoglucanase having a Melting Point (DSC) above 70° C., may be added before and/or during liquefaction step i). Optionally a protease, a carbohydrate-source generating enzyme, preferably a glucoamylase, a pullulanase, and/or a phytase may be present and/or added as well. In a preferred embodiment, a composition of the invention defined below may suitably be used in liquefaction in a process of the invention. The enzymes may be added individually or as one or more blend compositions comprising an alpha-amylase, the xylanase preferably having a Melting Point (DSC) above 80° C., and optional endoglucanase and optionally a protease, a carbohydrate-source generating enzyme, a pullulanase and/or a phytase.

Examples of alpha-amylases can be found in the “Alpha-Amylase Present and/or Added During Liquefaction”—section below.

In an embodiment the alpha-amylase is a variant of the alpha-amylase shown in SEQ ID NO: 5 herein, such as one derived from a strain Bacillus stearomthermphilus, with mutations selected from the group of: —I181*+G182*; —I181*+G182*+N193F; preferably —I181*+G182*+E129V+K177L+R179E; —I181*+G182*+N193F+E129V+K177L+R179E; —I181*+G182*+N193F+V59A+Q89R+E129V+K177L+R179E+H208Y+K220P+N224L+Q254S; —I181*+G182*+N193F+V59A+Q89R+E129V+K177L+R179E+Q254S+M284V; —I181*+G182*+N193F+E129V+K177L+R179E+K220P+N224L+S242Q+Q254S; and —I181*+G182*+N193F+E129V+K177L+R179S+K220P+N224L+S242Q+Q254S (using SEQ ID NO: 5 herein for numbering).

Bacillus stearothermophilus alpha-amylases are typically naturally truncated when produced to be around 491 amino acids long (compared to SEQ ID NO: 3 in WO 99/19467 or SEQ ID NO: 5 herein), such as from about 480-495 amino acids long.

In an embodiment, the bacterial alpha-amylase, e.g., Bacillus alpha-amylase, such as especially Bacillus stearothermophilus alpha-amylase, is dosed in liquefaction in a concentration between 0.01-10 KNU-A/g DS, e.g., between 0.02 and 5 KNU-A/g DS, such as 0.03 and 3 KNU-A, preferably 0.04 and 2 KNU-A/g DS, such as especially 0.01 and 2 KNU-A/g DS.

In an embodiment, the bacterial alpha-amylase, e.g., Bacillus alpha-amylase, such as especially Bacillus stearothermophilus alpha-amylase, is dosed in liquefaction in a concentration of between 0.0001-1 mg EP (Enzyme Protein)/g DS, e.g., 0.0005-0.5 mg EP/g DS, such as 0.001-0.1 mg EP/g DS.

In one aspect, a GH10 xylanase from Thermotoga containing the motif YITEMD (SEQ ID NO: 30) is used in a process or composition of the invention. Examples of suitable thermostable xylanases, in particular xylanases from the genus Thermotoga, include the xylanase shown in SEQ ID NOs: 2 herein, e.g., derived from a strain of Thermotoga maritima; the xylanase shown in SEQ ID NO: 3 herein, e.g., derived from a strain of Thermotoga neapolitana; the xylanase shown in SEQ ID NO: 4 herein, e.g., derived from a strain of Thermotoga naphthophila; or polypeptides having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity, such as 100% identity to the mature part of any of the polypeptides of SEQ ID NOs: 2, 3, and 4 herein, respectively.

In an embodiment, the thermostable metal-ion inhibition resistant xylanase is a GH10 family xylanase from Thermotoga containing the motif YITEMD (SEQ ID NO: 30) and further having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity, such as 100% identity to the mature part of any of the polypeptides of SEQ ID NOs: 2.

In an embodiment, the thermostable metal-ion inhibition resistant xylanase is a GH10 family xylanase from Thermotoga containing the motif YITEMD (SEQ ID NO: 30) and further having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity, such as 100% identity to the mature part of any of the polypeptides of SEQ ID NOs: 3.

In an embodiment, the thermostable metal-ion inhibition resistant xylanase is a GH10 family xylanase from Thermotoga containing the motif YITEMD (SEQ ID NO: 30) and further having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity, such as 100% identity to the mature part of any of the polypeptides of SEQ ID NOs: 4.

Examples of suitable optional endoglucanases having a Melting Point (DSC) above 70° C. can be found in the “Thermostable Endoglucanase Present and/or Added During Liquefaction”—section below.

In a preferred embodiment, the endoglucanase is the one shown in SEQ ID NO: 7 herein, such as one derived from a strain of Talaromyces leycettanus (WO2013/019780), or an endoglucanase having at least 80%, at least 85%, at least 90%, at least 91%, at least 92%, at least 93%, at least 94%, at least 95%, at least 96%, at least 97%, at least 98%, or at least 99% identity to SEQ ID NO: 7 herein.

In a preferred embodiment, the endoglucanase is the one shown in SEQ ID NO: 7 herein, such as one derived from a strain of Talaromyces leycettanus (WO2013/019780—hereby incorporated by reference), or an endoglucanase having at least 80%, at least 85%, at least 90%, at least 91%, at least 92%, at least 93%, at least 94%, at least 95%, at least 96%, at least 97%, at least 98%, or at least 99% identity to SEQ ID NO: 7 herein having a Melting Point (DSC) above 70° C.

Examples of optional proteases can be found in the “Protease Present and/or Added During Liquefaction”—section below.

Examples of suitable optional carbohydrate-source generating enzymes, preferably thermostable carbohydrate-source generating enzymes, in particular glucoamylases, can be found in the “Carbohydrate-Source Generating Enzymes Present and/or Added During Liquefaction”—section below.

A suitable optional pullulanase can be found in the “Pullulanase Present and/or Added During Liquefaction”—section below. In a preferred embodiment, the pullulanase is derived from Bacillus sp.

Examples of optional phytases can be found in the “Phytase Present and/or Added During Liquefaction”—section below. In a preferred embodiment, the phytase is derived from a strain of Buttiauxella.

A suitable cellulase or cellulolytic enzyme composition present and/or added during saccharification and/or fermentation or simultaneous saccharification and fermentation (SSF) can be found in the “Cellulase or Cellulolytic Enzyme Composition Present and/or Added During Saccharification and/or Fermentation or SSF”—section below. In an embodiment, the cellulase or cellulolytic enzyme composition is derived from Trichoderma reesei.

In a preferred embodiment, the cellulase or cellulolytic enzyme composition is a Trichoderma reesei cellulolytic enzyme composition, further comprising Thermoascus aurantiacus GH61A polypeptide having cellulolytic enhancing activity (SEQ ID NO: 6 in WO 2005/074656 or SEQ ID NO: 18 herein) and Aspergillus fumigatus beta-glucosidase (SEQ ID NO: 2 of WO 2005/047499 or SEQ ID NO: 16 herein).

In an embodiment, the cellulase or cellulolytic enzyme composition is a Trichoderma reesei cellulolytic enzyme composition further comprising Penicillium emersonii GH61A polypeptide disclosed as SEQ ID NO: 2 in WO 2011/041397 or SEQ ID NO: 19 herein, and Aspergillus fumigatus beta-glucosidase disclosed as SEQ ID NO: 2 in WO 2005/047499 or SEQ ID NO: 16 herein, or a variant thereof, which variant has one of, preferably all of, the following substitutions: F100D, S283G, N456E, F512Y (using SEQ ID NO: 16 herein for numbering).

In an embodiment, the cellulase or cellulolytic enzyme composition is a Trichoderma reesei cellulolytic enzyme composition further comprising Aspergillus fumigatus beta-glucosidase disclosed as SEQ ID NO: 2 in WO 2005/047499 or SEQ ID NO: 16 herein, or a variant thereof, which variant has one of, preferably all of the following substitutions: F100D, S283G, N456E, F512Y (using SEQ ID NO: 16 herein for numbering).

According to the process of the invention the pH during liquefaction may be between 4.0-6.5, such as 4.5-6.2, such as above 4.8-6.0, such as between 5.0-5.8.

According to the invention the temperature is above the initial gelatinization temperature. The term “initial gelatinization temperature” refers to the lowest temperature at which solubilization of starch, typically by heating, begins. The temperature can vary for different starches. The initial gelanitization temperature may be from 50-70° C.

In an embodiment, the temperature during liquefaction step i) is in the range from 70-100° C., such as between 70-95° C., such as between 75-90° C., preferably between 80-90° C., such as around 85° C.

In an embodiment, the process of the invention further comprises, prior to the step i), the steps of:

a) reducing the particle size of the starch-containing material, preferably by dry milling;

b) forming a slurry comprising the starch-containing material and water.

The starch-containing starting material, such as whole grains, may be reduced in particle size, e.g., by milling, in order to open up the structure, to increase the surface area and allowing for further processing. Generally, there are two types of processes: wet and dry milling. In dry milling whole kernels are milled and used. Wet milling gives a good separation of germ and meal (starch granules and protein). Wet milling is often applied at locations where the starch hydrolysate is used in production of, e.g., syrups. Both dry and wet millings are well known in the art of starch processing. According to the present invention dry milling is preferred. The particle size may be reduced even further, for example, by Turkish grinding. In an embodiment, the particle size is reduced to between 0.05 to 3.0 mm, preferably 0.1-0.5 mm, or so that at least 30%, preferably at least 50%, more preferably at least 70%, even more preferably at least 90% of the starch-containing material fit through a sieve with a 0.05 to 3.0 mm screen, preferably 0.1-0.5 mm screen. In another embodiment at least 50%, preferably at least 70%, more preferably at least 80%, especially at least 90% of the starch-containing material fit through a sieve with #6 screen. In an embodiment, at least 75% of the starch-containing material fit through a sieve with less than a 0.5 mm screen, more preferably at least 79% or 80% of the starch-containing material fit through a sieve with less than a 0.425 mm screen. In an embodiment, at least 50% of the starch-containing material fit through a sieve with a 0.25 to 0.425 mm screen, more preferably at least 59% or 60% of the starch-containing material fit through a sieve with a 0.25 to 0.425 mm screen.

The aqueous slurry may contain from 10-55 w/w-% dry solids (DS), preferably 25-45 w/w-% dry solids (DS), more preferably 30-40 w/w-% dry solids (DS) of starch-containing material.

The slurry may be heated to above the initial gelatinization temperature, preferably to between 70-95° C., such as between 80-90° C., between pH 5.0-7.0, preferably between 5.0 and 6.0, for 30 minutes to 5 hours, such as around 2 hours.

In an embodiment liquefaction step i) is carried out for 0.5-5 hours at a temperature from 70-95° C. at a pH from 4-6.

In a preferred embodiment liquefaction step i) is carried out for 0.5-3 hours at a temperature from 80-90° C. at a pH from 4-6.

The alpha-amylase, metal-ion inhibition resistant xylanase, and optional thermostable endoglucanase, optional protease, optional carbohydrate-source generating enzyme, in particular glucoamylase, optional pullulanase, and/or optional phytase, may initially be added to the aqueous slurry to initiate liquefaction (thinning). In an embodiment only a portion of the enzymes is added to the aqueous slurry, while the rest of the enzymes are added during liquefaction step i).

The aqueous slurry may in an embodiment be jet-cooked to further gelatinize the slurry before being subjected to liquefaction in step i). The jet-cooking may be carried out at a temperature between 95-160° C., such as between 110-145° C., preferably 120-140° C., such as 125-135° C., preferably around 130° C. for about 1-15 minutes, preferably for about 3-10 minutes, especially around about 5 minutes.

### Saccharification and Fermentation

According to the process of the invention one or more carbohydrate-source generating enzymes, in particular glucoamylase, may be present and/or added during saccharification step ii) and/or fermentation step iii). The carbohydrate-source generating enzyme may preferably be a glucoamylase, but may also be an enzyme selected from the group consisting of: beta-amylase, maltogenic amylase and alpha-glucosidase. The carbohydrate-source generating enzyme added during saccharification step ii) and/or fermentation step iii) is typically different from the optional carbohydrate-source generating enzyme, in particular glucoamylase, optionally added during liquefaction step i). In an embodiment the carbohydrate-source generating enzymes, in particular glucoamylase, is added together with a fungal alpha-amylase. Examples of carbohydrate-source generating enzymes, including glucoamylases, can be found in the “Carbohydrate-Source Generating Enzyme Present and/or Added During Saccharification and/or Fermentation”—section below.

When doing sequential saccharification and fermentation, saccharification step ii) may be carried out at conditions well-known in the art. For instance, the saccharification step ii) may last up to from about 24 to about 72 hours.

In an embodiment, a pre-saccharification step is done. In an embodiment, a carbohydrate-source generating enzyme is added during pre-saccharification carried out before saccharification step ii) and/or fermentation step iii). The carbohydrate-source generating enzyme may also be added during pre-saccharification carried out before simultaneous saccharification and fermentation (SSF).

In an embodiment, a carbohydrate-source generating enzyme, preferably glucoamylase, and/or the cellulase or cellulolytic enzyme composition, are added during pre-saccharification carried out before saccharification step ii) and/or fermentation step iii). The carbohydrate-source generating enzyme, preferably glucoamylase, and the cellulase or cellulolytic enzyme composition may also be added during pre-saccharification carried out before simultaneous saccharification and fermentation (SSF). Pre-saccharification is typically done for 40-90 minutes at a temperature between 30-65° C., typically around 60° C. Pre-saccharification may be followed by saccharification during fermentation in simultaneous saccharification and fermentation (“SSF). Saccharification is typically carried out at temperatures from 20-75° C., preferably from 40-70° C., typically around 60° C., and at a pH between 4 and 5, such as around pH 4.5.

Simultaneous saccharification and fermentation (“SSF”) is widely used in industrial scale fermentation product production processes, especially ethanol production processes. When doing SSF the saccharification step ii) and the fermentation step iii) are carried out simultaneously. There may be no holding stage for the saccharification, meaning that a fermenting organism, such as yeast, and enzyme(s), in a preferred embodiment according to the invention a glucoamylase and a cellulase or cellulolytic enzyme composition, may be added together. However, it is also contemplated to add the fermenting organism and enzyme(s) separately. Fermentation or SSF may, according to the invention, typically be carried out at a temperature from 25° C. to 40° C., such as from 28° C. to 35° C., such as from 30° C. to 34° C., preferably around about 32° C. In an embodiment fermentation is ongoing for 6 to 120 hours, in particular 24 to 96 hours. In an embodiment the pH is between 3.5-5, in particular between 3.8 and 4.3.

### Fermentation Medium

“Fermentation media” or “fermentation medium” refers to the environment in which fermentation is carried out. The fermentation medium includes the fermentation substrate, that is, the carbohydrate source that is metabolized by the fermenting organism. According to the invention the fermentation medium may comprise nutrients and growth stimulator(s) for the fermenting organism(s). Nutrient and growth stimulators are widely used in the art of fermentation and include nitrogen sources, such as ammonia; urea, vitamins and minerals, or combinations thereof.

### Fermenting Organisms

The term “fermenting organism” refers to any organism, including bacterial and fungal organisms, especially yeast, suitable for use in a fermentation process and capable of producing the desired fermentation product. Especially suitable fermenting organisms are able to ferment, i.e., convert, sugars, such as glucose or maltose, directly or indirectly into the desired fermentation product, such as ethanol. Examples of fermenting organisms include fungal organisms, such as yeast. Preferred yeast includes strains of Saccharomyces spp., in particular, Saccharomyces cerevisiae.

Suitable concentrations of the viable fermenting organism during fermentation, such as SSF, are well known in the art or can easily be determined by the skilled person in the art. In one embodiment the fermenting organism, such as ethanol fermenting yeast, (e.g., Saccharomyces cerevisiae) is added to the fermentation medium so that the viable fermenting organism, such as yeast, count per mL of fermentation medium is in the range from 10⁵ to 10¹², preferably from 10⁷ to 10¹⁰, especially about 5×10⁷.

Examples of commercially available yeast includes, e.g., RED START™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

### Starch-Containing Materials

Any suitable starch-containing material may be used according to the present invention. The starting material is generally selected based on the desired fermentation product. Examples of starch-containing materials, suitable for use in a process of the invention, include whole grains, corn, wheat, barley, rye, milo, sago, cassava, tapioca, sorghum, rice, peas, beans, or sweet potatoes, or mixtures thereof or starches derived therefrom, or cereals. Contemplated are also waxy and non-waxy types of corn and barley.

In a preferred embodiment, the starch-containing material, used for ethanol production according to the invention, is corn or wheat.

### Fermentation Products

The term “fermentation product” means a product produced by a process including a fermentation step using a fermenting organism. Fermentation products contemplated according to the invention include alcohols (e.g., ethanol, methanol, butanol; polyols such as glycerol, sorbitol and inositol); organic acids (e.g., citric acid, acetic acid, itaconic acid, lactic acid, succinic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment, the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferably processes of the invention are used for producing an alcohol, such as ethanol. The fermentation product, such as ethanol, obtained according to the invention, may be used as fuel, which is typically blended with gasoline. However, in the case of ethanol it may also be used as potable ethanol.

### Recovery

Subsequent to fermentation, or SSF, the fermentation product may be separated from the fermentation medium. The slurry may be distilled to extract the desired fermentation product (e.g., ethanol). Alternatively the desired fermentation product may be extracted from the fermentation medium by micro or membrane filtration techniques. The fermentation product may also be recovered by stripping or other method well known in the art.

Alpha-Amylase Present and/or Added During Liquefaction

According to the invention an alpha-amylase is present and/or added in liquefaction together with the metal-ion inhibition resistant xylanase, preferably having a Melting Point (DSC) above 80° C., such as between 80° C. and 95° C., and an optional endoglucanase, an optional protease, an optional carbohydrate-source generating enzyme, in particular a glucoamylase, an optional pullulanase, and/or an optional phytase.

The alpha-amylase added during liquefaction step i) may be any alpha-amylase. Preferred are bacterial alpha-amylases, such as especially Bacillus alpha-amylases, such as Bacillus stearothermophilus alpha-amylases, which are stable at temperature used during liquefaction.

### Bacterial Alpha-Amylase

The term “bacterial alpha-amylase” means any bacterial alpha-amylase classified under EC 3.2.1.1. A bacterial alpha-amylase used according to the invention may, e.g., be derived from a strain of the genus Bacillus, which is sometimes also referred to as the genus Geobacillus. In an embodiment, the Bacillus alpha-amylase is derived from a strain of Bacillus amyloliquefaciens, Bacillus licheniformis, Bacillus stearothermophilus, Bacillus sp. TS-23, or Bacillus subtilis, but may also be derived from other Bacillus sp.

Specific examples of bacterial alpha-amylases include the Bacillus stearothermophilus alpha-amylase of SEQ ID NO: 3 in WO 99/19467 or SEQ ID NO: 5 herein, the Bacillus amyloliquefaciens alpha-amylase of SEQ ID NO: 5 in WO 99/19467, and the Bacillus licheniformis alpha-amylase of SEQ ID NO: 4 in WO 99/19467 and the Bacillus sp. TS-23 alpha-amylase disclosed as SEQ ID NO: 1 in WO 2009/061380 (all sequences are hereby incorporated by reference).

In an embodiment, the bacterial alpha-amylase may be an enzyme having a degree of identity of at least 60%, e.g., at least 70%, at least 80%, at least 90%, at least 95%, at least 96%, at least 97%, at least 98% or at least 99% to any of the sequences shown in SEQ ID NOS: 3, 4 or 5, respectively, in WO 99/19467 and SEQ ID NO: 1 in WO 2009/061380.

In an embodiment, the alpha-amylase may be an enzyme having a degree of identity of at least 60%, e.g., at least 70%, at least 80%, at least 90%, at least 91%, at least 92%, at least 93%, at least 94%, at least 95%, at least 96%, at least 97%, at least 98% or at least 99% to any of the sequences shown in SEQ ID NO: 3 in WO 99/19467 or SEQ ID NO: 5 herein.

In a preferred embodiment, the alpha-amylase is derived from Bacillus stearothermophilus. The Bacillus stearothermophilus alpha-amylase may be a mature wild-type or a mature variant thereof. The mature Bacillus stearothermophilus alpha-amylases, or variant thereof, may be naturally truncated during recombinant production. For instance, the mature Bacillus stearothermophilus alpha-amylase may be truncated at the C-terminal so it is around 491 amino acids long (compared to SEQ ID NO: 3 in WO 99/19467 or SEQ ID NO: 1 herein), such as from 480-495 amino acids long.

The Bacillus alpha-amylase may also be a variant and/or hybrid. Examples of such a variant can be found in any of WO 96/23873, WO 96/23874, WO 97/41213, WO 99/19467, WO 00/60059, WO 02/10355 and WO2009/061380 (all documents are hereby incorporated by reference). Specific alpha-amylase variants are disclosed in U.S. Pat. Nos. 6,093,562, 6,187,576, 6,297,038, and 7,713,723 (hereby incorporated by reference) and include Bacillus stearothermophilus alpha-amylase (often referred to as BSG alpha-amylase) variants having a deletion of one or two amino acids at any of positions R179, G180, I181 and/or G182, preferably the double deletion disclosed in WO 96/23873—see, e.g., page 20, lines 1-10 (hereby incorporated by reference), preferably corresponding to deletion of positions I181 and G182 compared to the amino acid sequence of Bacillus stearothermophilus alpha-amylase set forth in SEQ ID NO: 3 disclosed in WO 99/19467 or SEQ ID NO: 5 herein or the deletion of amino acids R179 and G180 using SEQ ID NO: 3 in WO 99/19467 or SEQ ID NO: 5 herein. Even more preferred are Bacillus alpha-amylases, especially Bacillus stearothermophilus (BSG) alpha-amylases, which have at one or two amino acid deletions corresponding to positions R179, G180, I181 and G182, preferably which have a double deletion corresponding to R179 and G180, or preferably a deletion of positions 181 and 182 (denoted I181*+G182*), and optionally further comprises a N193F substitution (denoted I181*+G182*+N193F) compared to the wild-type BSG alpha-amylase amino acid sequence set forth in SEQ ID NO: 3 disclosed in WO 99/19467 or SEQ ID NO: 5 herein. The bacterial alpha-amylase may also have a substitution in a position corresponding to S239 in the Bacillus licheniformis alpha-amylase shown in SEQ ID NO: 4 in WO 99/19467, or a S242 variant in the Bacillus stearothermophilus alpha-amylase of SEQ ID NO: 3 in WO 99/19467 or SEQ ID NO: 5 herein.

In an embodiment, the variant is a S242A, E or Q variant, preferably a S242Q or A variant, of the Bacillus stearothermophilus alpha-amylase (using SEQ ID NO: 5 herein for numbering).

In an embodiment, the variant is a position E188 variant, preferably E188P variant of the Bacillus stearothermophilus alpha-amylase (using SEQ ID NO: 5 herein for numbering).

Other contemplated variants are Bacillus sp. TS-23 variant disclosed in WO2009/061380, especially variants defined in claim 1 of WO2009/061380 (hereby incorporated by reference).

### Bacterial Hybrid Alpha-Amylases

The bacterial alpha-amylase may also be a hybrid bacterial alpha-amylase, e.g., an alpha-amylase comprising 445 C-terminal amino acid residues of the Bacillus licheniformis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467). In a preferred embodiment, this hybrid has one or more, especially all, of the following substitutions: G48A+T49I+G107A+H156Y+A181T+N190F+1201F+A209V+Q264S (using the Bacillus licheniformis numbering in SEQ ID NO: 4 of WO 99/19467). Also preferred are variants having one or more of the following mutations (or corresponding mutations in other Bacillus alpha-amylases): H154Y, A181T, N190F, A209V and Q264S and/or the deletion of two residues between positions 176 and 179, preferably the deletion of E178 and G179 (using SEQ ID NO: 5 of WO 99/19467 for position numbering).

In an embodiment the bacterial alpha-amylase is the mature part of the chimeric alpha-amylase disclosed in Richardson et al., 2002, The Journal of Biological Chemistry 277(29): 267501-26507, referred to as BD5088 or a variant thereof. This alpha-amylase is the same as the one shown in SEQ ID NO: 2 in WO 2007134207. The mature enzyme sequence starts after the initial “Met” amino acid in position 1.

### Thermostable Alpha-Amylase

According to the invention a thermostable alpha-amylase can be used in liquefying step i) in combination with the thermostable xylanase that is resistant to inhibition due to metal ions in, preferably having a Melting Point (DSC) above 80° C. The thermostable alpha-amylase may be added together with an optional carbohydrate-source generating enzyme, in particular a thermostable glucoamylase, and/or optional pullulanase. Optionally an endoglucanase having a Melting Point (DSC) above 70° C., such as above 75° C., in particular above 80° C. may be included. The thermostable alpha-amylase, such as a bacterial an alpha-amylase, is preferably derived from Bacillus stearothermophilus or Bacillus sp. TS-23. In an embodiment the alpha-amylase has a T½ (min) at pH 4.5, 85° C., 0.12 mM CaCl₂ of at least 10.

The term “bacterial alpha-amylase” means any bacterial alpha-amylase classified under EC 3.2.1.1. A bacterial alpha-amylase used according to the invention may, e.g., be derived from a strain of the genus Bacillus, which is sometimes also referred to as the genus Geobacillus. In an embodiment the Bacillus alpha-amylase is derived from a strain of Bacillus amyloliquefaciens, Bacillus licheniformis, Bacillus stearothermophilus, or Bacillus subtilis, but may also be derived from other Bacillus sp.

Specific examples of bacterial alpha-amylases include the Bacillus stearothermophilus alpha-amylase of SEQ ID NO: 3 in WO 99/19467, the Bacillus amyloliquefaciens alpha-amylase of SEQ ID NO: 5 in WO 99/19467, and the Bacillus licheniformis alpha-amylase of SEQ ID NO: 4 in WO 99/19467 (all sequences are hereby incorporated by reference). In an embodiment the alpha-amylase may be an enzyme having a degree of identity of at least 60%, e.g., at least 70%, at least 80%, at least 90%, at least 95%, at least 96%, at least 97%, at least 98% or at least 99% to any of the sequences shown in SEQ ID NOS: 3, 4 or 5, respectively, in WO 99/19467.

In an embodiment the alpha-amylase may be an enzyme having a degree of identity of at least 60%, e.g., at least 70%, at least 80%, at least 90%, at least 91%, at least 92%, at least 93%, at least 94%, at least 95%, at least 96%, at least 97%, at least 98% or at least 99% to any of the sequences shown in SEQ ID NO: 3 in WO 99/19467 or SEQ ID NO: 5 herein.

In a preferred embodiment the alpha-amylase is derived from Bacillus stearothermophilus. The Bacillus stearothermophilus alpha-amylase may be a mature wild-type or a mature variant thereof. The mature Bacillus stearothermophilus alpha-amylases may naturally be truncated during recombinant production. For instance, the Bacillus stearothermophilus alpha-amylase may be a truncated so it has around 491 amino acids compared to SEQ ID NO: 3 in WO 99/19467.

The Bacillus alpha-amylase may also be a variant and/or hybrid. Examples of such a variant can be found in any of WO 96/23873, WO 96/23874, WO 97/41213, WO 99/19467, WO 00/60059, and WO 02/10355 (all documents are hereby incorporated by reference). Specific alpha-amylase variants are disclosed in U.S. Pat. Nos. 6,093,562, 6,187,576, 6,297,038, and 7,713,723 (hereby incorporated by reference) and include Bacillus stearothermophilus alpha-amylase (often referred to as BSG alpha-amylase) variants having a deletion of one or two amino acids at positions R179, G180, I181 and/or G182, preferably a double deletion disclosed in WO 96/23873—see, e.g., page 20, lines 1-10 (hereby incorporated by reference), preferably corresponding to deletion of positions I181 and G182 compared to the amino acid sequence of Bacillus stearothermophilus alpha-amylase set forth in SEQ ID NO: 3 disclosed in WO 99/19467 or SEQ ID NO: 5 herein or the deletion of amino acids R179 and G180 using SEQ ID NO: 3 in WO 99/19467 or SEQ ID NO: 5 herein for numbering (which reference is hereby incorporated by reference). Even more preferred are Bacillus alpha-amylases, especially Bacillus stearothermophilus alpha-amylases, which have a double deletion corresponding to a deletion of positions 181 and 182 and further comprise a N193F substitution (also denoted I181*+G182*+N193F) compared to the wild-type BSG alpha-amylase amino acid sequence set forth in SEQ ID NO: 3 disclosed in WO 99/19467 or SEQ ID NO: 5 herein. The bacterial alpha-amylase may also have a substitution in a position corresponding to S239 in the Bacillus licheniformis alpha-amylase shown in SEQ ID NO: 4 in WO 99/19467, or a S242 variant of the Bacillus stearothermophilus alpha-amylase of SEQ ID NO: 3 in WO 99/19467 or SEQ ID NO: 5 herein.

In an embodiment the variant is a S242A, E or Q variant, preferably a S242Q variant, of the Bacillus stearothermophilus alpha-amylase (using SEQ ID NO: 5 herein for numbering).

In an embodiment the variant is a position E188 variant, preferably E188P variant of the Bacillus stearothermophilus alpha-amylase (using SEQ ID NO: 5 herein for numbering).

The bacterial alpha-amylase may in an embodiment be a truncated alpha-amylase. Especially the truncation is so that the Bacillus stearothermophilus alpha-amylase shown in SEQ ID NO: 3 in WO 99/19467 or SEQ ID NO: 5 herein, is around 491 amino acids long, such as from 480 to 495 amino acids long.

Most importantly, a suitable alpha-amylase for use in liquefaction must have sufficient therm-stability, and thus accordingly any alpha-amylase having a T½ (min) at pH 4.5, 85° C., 0.12 mM CaCl₂) of at least 10, such as at least 15, such as at least 20, such as at least 25, such as at least 30, such as at least 40, such as at least 50, such as at least 60, such as between 10-70, such as between 15-70, such as between 20-70, such as between 25-70, such as between 30-70, such as between 40-70, such as between 50-70, such as between 60-70, may be used.

According to the invention the alpha-amylase may be a thermostable alpha-amylase, such as a thermostable bacterial alpha-amylase, preferably from Bacillus stearothermophilus. In an embodiment the alpha-amylase used according to the invention has a T½ (min) at pH 4.5, 85° C., 0.12 mM CaCl₂ of at least 10.

In an embodiment the thermostable alpha-amylase has a T½ (min) at pH 4.5, 85° C., 0.12 mM CaCl₂, of at least 15.

In an embodiment the thermostable alpha-amylase has a T½ (min) at pH 4.5, 85° C., 0.12 mM CaCl₂, of as at least 20.

In an embodiment the thermostable alpha-amylase has a T½ (min) at pH 4.5, 85° C., 0.12 mM CaCl₂, of as at least 25.

In an embodiment the thermostable alpha-amylase has a T½ (min) at pH 4.5, 85° C., 0.12 mM CaCl₂, of as at least 30.

In an embodiment the thermostable alpha-amylase has a T½ (min) at pH 4.5, 85° C., 0.12 mM CaCl₂, of as at least 40.

In an embodiment the thermostable alpha-amylase has a T½ (min) at pH 4.5, 85° C., 0.12 mM CaCl₂, of at least 50.

In an embodiment the thermostable alpha-amylase has a T½ (min) at pH 4.5, 85° C., 0.12 mM CaCl₂, of at least 60.

In an embodiment the thermostable alpha-amylase has a T½ (min) at pH 4.5, 85° C., 0.12 mM CaCl₂, between 10-70.

In an embodiment the thermostable alpha-amylase has a T½ (min) at pH 4.5, 85° C., 0.12 mM CaCl₂, between 15-70.

In an embodiment the thermostable alpha-amylase has a T½ (min) at pH 4.5, 85° C., 0.12 mM CaCl₂, between 20-70.

In an embodiment the thermostable alpha-amylase has a T½ (min) at pH 4.5, 85° C., 0.12 mM CaCl₂, between 25-70.

In an embodiment the thermostable alpha-amylase has a T½ (min) at pH 4.5, 85° C., 0.12 mM CaCl₂, between 30-70.

In an embodiment the thermostable alpha-amylase has a T½ (min) at pH 4.5, 85° C., 0.12 mM CaCl₂, between 40-70.

In an embodiment the thermostable alpha-amylase has a T½ (min) at pH 4.5, 85° C., 0.12 mM CaCl₂, between 50-70.

In an embodiment the thermostable alpha-amylase has a T½ (min) at pH 4.5, 85° C., 0.12 mM CaCl₂, between 60-70.

In an embodiment of the invention the alpha-amylase is a bacterial alpha-amylase, preferably derived from the genus Bacillus, especially a strain of Bacillus stearothermophilus, in particular the Bacillus stearothermophilus as disclosed in WO 99/019467 as SEQ ID NO: 3 (SEQ ID NO: 5 herein) with one or two amino acids deleted at positions R179, G180, I181 and/or G182, in particular with R179 and G180 deleted, or with I181 and G182 deleted, with mutations in below list of mutations.

In preferred embodiments the Bacillus stearothermophilus alpha-amylases have double deletion I181+G182, and optional substitution N193F, further comprising mutations selected from below list.

In a preferred embodiment the alpha-amylase is selected from the following group of Bacillus stearothermophilus alpha-amylase variants (using SEQ ID NO: 5 for numbering):

### I181*+G182*+N193F+E129V+K177L+R179E;

### I181*+G182*+N193F+V59A+Q89R+E129V+K177L+R179E+H208Y+K220P+N224L+Q254S

### I181*+G182*+N193F+V59A+Q89R+E129V+K177L+R179E+Q254S+M284V;

### I181*+G182*+N193F+V59A+E129V+K177L+R179E+Q254S+M284V;

### I181*+G182*+N193F+E129V+K177L+R179E+K220P+N224L+5242Q+Q254S;

### I181*+G182*+V59A+E129V+K177L+R179E+Q254S+M284V+V212T+Y268G+N293Y+T297N;

### I181*+G182*+V59A+E129V+K177L+R179E+Q254S+M284V+V212T+Y268G+N293Y+T297N+S173N+E188P+H208Y+S242Y+K279I;

### I181*+G182*+V59A+E129V+K177L+R179S+Q254S+M284V+V212T+Y268G+N293Y+T297N+A184Q+E188P+T191N

### I181*+G182*+V59A+E129V+K177L+R179S+Q254S+M284V+V212T+Y268G+N293Y+T297N+A184Q+E188P+T191N+S242Y+K279I;

### I181*+G182*+V59A+E129V+K177L+R179S+Q254S+M284V+V212T+Y268G+N293Y+T297N+A184Q+E188P+T191N+N193F+S242Y+K279I;

### I181*+G182*+V59A+E129V+K177L+R179E+Q254S+M284V+V212T+Y268G+N293Y+T297N+E188P+K279W;

### I181*+G182*+V59A+E129V+K177L+R179E+Q254S+M284V+V212T+Y268G+N293Y+T297N+W115D+D117Q+T133P; and

wherein the variant has at least 80%, at least 85%, at least 90%, at least 95%, at least 96%, at least 97%, at least 98%, or at least 99% sequence identity to SEQ ID NO: 5.

It should be understood, that when referring to Bacillus stearothermophilus alpha-amylase and variants thereof they are normally produced in truncated form. In particular, the truncation may be so that the Bacillus stearothermophilus alpha-amylase shown in SEQ ID NO: 3 in WO 99/19467 or SEQ ID NO: 5 herein, or variants thereof, are truncated in the C-terminal and are typically around 491 amino acids long, such as from 480-495 amino acids long.

In a preferred embodiment the alpha-amylase variant may be an enzyme having a degree of identity of at least 60%, e.g., at least 70%, at least 80%, at least 85%, at least 90%, at least 95%, at least 91%, at least 92%, at least 93%, at least 94%, at least 95%, at least 96%, at least 97%, at least 98% or at least 99%, but less than 100% to the sequence shown in SEQ ID NO: 3 in WO 99/19467 or SEQ ID NO: 5 herein.

Thermostable Metal Ion Inhibition Resistant Xylanases Present and/or Added During Liquefaction

According to the invention a metal ion inhibition resistant xylanase, preferably having a Melting Point (DSC) above 80° C. is present and/or added to liquefaction step i), for example in combination with an alpha-amylase, such as a bacterial alpha-amylase (described above). The phrases “metal ion inhibition resistant xylanase” and “xylanase that is resistant to inhibition by metal ions” are used interchangeably herein to refer to a xylanase that retains a significant amount of its relative activity in the presence of a metal ion compared to a xylanase that loses a significant amount of its activity in the presence of the metal ion. For example, a xylanase that is not metal ion inhibition resistant will lose at least 40%, at least 50%, at least 55%, at least 60%, at least 65%, at least 70%, at least 71%, at least 72%, at least 73%, at least 74%, at least 75%, at least 76%, at least 77%, at least 78%, at least 79%, or at least 80% of its relative activity in the presence of a metal ion compared to its activity in the absence of the metal ion, in contrast to a metal ion inhibition resistant xylanase that will retain at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of its relative activity in the presence of the same metal ion compared to its activity in the absence of that metal ion. To determine whether a xylanase is metal ion inhibition resistant, the relative activity of the xylanase in the presence of one or more metal ions can be assays compared to its relative activity in the absence of those one metal ions.

A metal ion inhibition resistant xylanase may also not only retain 100% of its relative activity in the presence of a metal ion, but may also exhibit increased relative activity in the presence of a metal ion. For example, a metal ion inhibition resistant xylanase of the present invention may exhibit relative activity that increases by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% compared to the relative activity of the xylanase in the absence of the metal ion.

The thermostability of a xylanase may be determined as described in the “Materials & Methods”—section under “Determination of T_{d} by Differential Scanning Calorimetry for Xylanases”.

In an embodiment the metal ion inhibition resistant xylanase, especially from the genus Thermotoga has a Melting Point (DSC) above 82° C., such as above 84° C., such as above 86° C., such as above 88° C., such as above 88° C., such as above 90° C., such as above 92° C., such as above 94° C., such as above 96° C., such as above 98° C., such as above 100° C., such as between 80° C. and 110° C., such as between 82° C. and 110° C., such as between 84° C. and 110° C.

In an embodiment the metal ion inhibition resistant xylanase, especially GH10 family xylanases from Thermotoga, contain the motif YITEMD (SEQ ID NO: 30).

In an embodiment of the first, second, and third aspects, liquefying the starch-containing material in step i) using the thermostable xylanase from the genus Thermotoga increases the amount of short chain oligosaccharides at the end of liquefying step i) (e.g., the amount of short chain oligosacchardies in the liquefact) compared to the amount of short chain oligosaccharides at the end of liquefying step i) when using a non metal-ion inhibition resistant thermostable xylanase in liquefying step i), such as for example the Dt xylanase of SEQ ID NO: 1. In an embodiment, thermostable xylanases from the genus Thermotoga containing the motif YITEMD (SEQ ID NO: 30) are used in liquefaction to increase the amount of short chain oligosaccharides in the liquefact.

In an embodiment of the first, second, and third aspects, liquefying the starch-containing material in step i) using the thermostable xylanase from the genus Thermotoga decreases the amount of residual starch present at the end of liquefying step i) (e.g., the amount of short chain oligosacchardies in the liquefact) compared to the amount of residual starch present at the end of liquefying step i) when using a non metal-ion inhibition resistant thermostable xylanase in liquefying step i), such as for example the Dt xylanase of SEQ ID NO: 1. In an embodiment, thermostable xylanases from the genus Thermotoga containing the motif YITEMD (SEQ ID NO: 30) are used in liquefaction to decrease the amount of residual starch present at the end of liquefying step i).

In a preferred embodiment the metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, at least 99%, such as 100% identity to the mature part of the polypeptide of SEQ ID NO: 2 herein, preferably derived from a strain of the genus Thermotoga, such as a strain of Thermotoga maritima.

In an embodiment of the first, second, and third aspects, liquefying the starch-containing material in step i) using a thermostable xylanase having at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, at least 99%, such as 100% identity to the mature part of the polypeptide of SEQ ID NO: 2 herein, preferably derived from a strain of the genus Thermotoga, such as a strain of Thermotoga maritima, increases the amount of short chain oligosaccharides at the end of liquefying step i) (e.g., the amount of short chain oligosacchardies in the liquefact) compared to the amount of short chain oligosaccharides at the end of liquefying step i) when using a non metal-ion inhibition resistant thermostable xylanase in liquefying step i), such as for example the Dt xylanase of SEQ ID NO: 1.

In an embodiment of the first, second, and third aspects, liquefying the starch-containing material in step i) using a thermostable xylanase having at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, at least 99%, such as 100% identity to the mature part of the polypeptide of SEQ ID NO: 2 herein, preferably derived from a strain of the genus Thermotoga, such as a strain of Thermotoga maritima, decreases the amount of residual starch present at the end of liquefying step i) (e.g., the amount of short chain oligosacchardies in the liquefact) compared to the amount of residual starch present at the end of liquefying step i) when using a non metal-ion inhibition resistant thermostable xylanase in liquefying step i), such as for example the Dt xylanase of SEQ ID NO: 1.

In an embodiment, the metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, or at least 99% identity to the amino acid sequence of SEQ ID NO: 2, and retains at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of its relative activity in the presence of the average concentration of the metal ion in the liquefying starch-containing material.

In an embodiment, metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, or at least 99% identity to the amino acid sequence of SEQ ID NO: 2, and retains at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of its relative activity in the presence of copper ions in the liquefying starch-containing material.

In an embodiment, the metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, or at least 99% identity to the amino acid sequence of SEQ ID NO: 2, and retains at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of its relative activity in the presence of iron ions in the liquefying starch-containing material.

In an embodiment, the metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, or at least 99% identity to the amino acid sequence of SEQ ID NO: 2, and retains at least 90%, at least 91%, at least 92%, at least 93%, at least 94%, at least 95%, at least 96%, at least 97%, or at least 98% of its relative activity in the presence of zinc ions in the liquefying starch-containing material.

In an embodiment, the metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, or at least 99% identity to the amino acid sequence of SEQ ID NO: 2, and retains: (i) at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of its relative activity in the presence of up to 0.25 mM copper ions in the liquefying starch-containing material; (ii) at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of its relative activity in the presence of up to 0.125 mM iron ions in the liquefying starch-containing material; and/or (iii) at least 90%, at least 91%, at least 92%, at least 93%, at least 94%, at least 95%, at least 96%, at least 97%, or at least 98% of its relative activity in the presence of up to 0.25 mM zinc ions in the liquefying starch-containing material.

In an embodiment the metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, at least 99%, such as 100% identity to the mature part of the polypeptide of SEQ ID NO: 3 herein, preferably derived from a strain of the genus Thermotoga, such as a strain of Thermotoga neopolitana.

In an embodiment of the first, second, and third aspects, liquefying the starch-containing material in step i) using a thermostable xylanase having at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, at least 99%, such as 100% identity to the mature part of the polypeptide of SEQ ID NO: 3 herein, preferably derived from a strain of the genus Thermotoga, such as a strain of Thermotoga neopolitana, increases the amount of short chain oligosaccharides at the end of liquefying step i) (e.g., the amount of short chain oligosacchardies in the liquefact) compared to the amount of short chain oligosaccharides at the end of liquefying step i) when using a non metal-ion inhibition resistant thermostable xylanase in liquefying step i), such as for example the Dt xylanase of SEQ ID NO: 1.

In an embodiment of the first, second, and third aspects, liquefying the starch-containing material in step i) using a thermostable xylanase having at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, at least 99%, such as 100% identity to the mature part of the polypeptide of SEQ ID NO: 3 herein, preferably derived from a strain of the genus Thermotoga, such as a strain of Thermotoga neopolitana, decreases the amount of residual starch present at the end of liquefying step i) (e.g., the amount of short chain oligosacchardies in the liquefact) compared to the amount of residual starch present at the end of liquefying step i) when using a non metal-ion inhibition resistant thermostable xylanase in liquefying step i), such as for example the Dt xylanase of SEQ ID NO: 1.

In an embodiment, the metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, or at least 99% identity to the amino acid sequence of SEQ ID NO: 3, and retains at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of its relative activity in the presence of the average concentration of the metal ion in the liquefying starch-containing material.

In an embodiment, metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, or at least 99% identity to the amino acid sequence of SEQ ID NO: 3, and retains at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of its relative activity in the presence of copper ions in the liquefying starch-containing material.

In an embodiment, the metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, or at least 99% identity to the amino acid sequence of SEQ ID NO: 3, and retains at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of its relative activity in the presence of iron ions in the liquefying starch-containing material.

In an embodiment, the metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, or at least 99% identity to the amino acid sequence of SEQ ID NO: 3, and retains at least 90%, at least 91%, at least 92%, at least 93%, at least 94%, at least 95%, at least 96%, at least 97%, or at least 98% of its relative activity in the presence of zinc ions in the liquefying starch-containing material.

In an embodiment, the metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, or at least 99% identity to the amino acid sequence of SEQ ID NO: 3, and retains: (i) at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of its relative activity in the presence of up to 0.25 mM copper ions in the liquefying starch-containing material; (ii) at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of its relative activity in the presence of up to 0.125 mM iron ions in the liquefying starch-containing material; and/or (iii) at least 90%, at least 91%, at least 92%, at least 93%, at least 94%, at least 95%, at least 96%, at least 97%, or at least 98% of its relative activity in the presence of up to 0.25 mM zinc ions in the liquefying starch-containing material.

In an embodiment the metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, at least 99%, such as 100% identity to the mature part of the polypeptide of SEQ ID NO: 4 herein, preferably derived from a strain of the genus Thermotoga, such as a strain of Thermotoga naphthophila.

In an embodiment of the first, second, and third aspects, liquefying the starch-containing material in step i) using a thermostable xylanase having at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, at least 99%, such as 100% identity to the mature part of the polypeptide of SEQ ID NO: 4 herein, preferably derived from a strain of the genus Thermotoga, such as a strain of Thermotoga naphthophila, increases the amount of short chain oligosaccharides at the end of liquefying step i) (e.g., the amount of short chain oligosacchardies in the liquefact) compared to the amount of short chain oligosaccharides at the end of liquefying step i) when using a non metal-ion inhibition resistant thermostable xylanase in liquefying step i), such as for example the Dt xylanase of SEQ ID NO: 1.

In an embodiment of the first, second, and third aspects, liquefying the starch-containing material in step i) using a thermostable xylanase having at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, at least 99%, such as 100% identity to the mature part of the polypeptide of SEQ ID NO: 4 herein, preferably derived from a strain of the genus Thermotoga, such as a strain of Thermotoga naphthophila, decreases the amount of residual starch present at the end of liquefying step i) (e.g., the amount of short chain oligosacchardies in the liquefact) compared to the amount of residual starch present at the end of liquefying step i) when using a non metal-ion inhibition resistant thermostable xylanase in liquefying step i), such as for example the Dt xylanase of SEQ ID NO: 1.

In an embodiment, the metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, or at least 99% identity to the amino acid sequence of SEQ ID NO: 4, and retains at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of its relative activity in the presence of the average concentration of the metal ion in the liquefying starch-containing material.

In an embodiment, metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, or at least 99% identity to the amino acid sequence of SEQ ID NO: 4, and retains at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of its relative activity in the presence of copper ions in the liquefying starch-containing material.

In an embodiment, the metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, or at least 99% identity to the amino acid sequence of SEQ ID NO: 4, and retains at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of its relative activity in the presence of iron ions in the liquefying starch-containing material.

In an embodiment, the metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, or at least 99% identity to the amino acid sequence of SEQ ID NO: 4, and retains at least 90%, at least 91%, at least 92%, at least 93%, at least 94%, at least 95%, at least 96%, at least 97%, or at least 98% of its relative activity in the presence of zinc ions in the liquefying starch-containing material.

In an embodiment, the metal ion inhibition resistant xylanase has at least 60%, such as at least 70%, such as at least 75%, at least 76%, at least 77%, at least 78%, at least 79%, preferably at least 80%, at least 81%, at least 82%, at least 83%, at least 84%, more preferably at least 85%, at least 86%, at least 87%, at least 88%, at least 89%, more preferably at least 90%, at least 91%, at least 92%, at least 93%, at least 94%, or at least 95%, such as at least 96%, at least 97%, at least 98%, or at least 99% identity to the amino acid sequence of SEQ ID NO: 4, and retains: (i) at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of its relative activity in the presence of up to 0.25 mM copper ions in the liquefying starch-containing material; (ii) at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of its relative activity in the presence of up to 0.125 mM iron ions in the liquefying starch-containing material; and/or (iii) at least 90%, at least 91%, at least 92%, at least 93%, at least 94%, at least 95%, at least 96%, at least 97%, or at least 98% of its relative activity in the presence of up to 0.25 mM zinc ions in the liquefying starch-containing material.

Thermostable Endoglucanase Present and/or Added During Liquefaction

According to the invention an optional endoglucanase (“EG”) having a Melting Point (DSC) above 70° C., such as between 70° C. and 95° C. may be present and/or added in liquefaction step i) in combination with a metal ion inhibition resistant xylanase, preferably having a Melting Point (DSC) above 80° C., and optionally a thermostable alpha-amylase, endoglucanase, carbohydrate-source generating enzyme, in particular a glucoamylase, optionally a pullulanase and/or optionally a phytase.

The thermostability of an endoglucanase may be determined as described in the “Materials & Methods”—section.

In an embodiment the endoglucanase has a Melting Point (DSC) above 72° C., such as above 74° C., such as above 76° C., such as above 78° C., such as above 80° C., such as above 82° C., such as above 84° C., such as above 86° C., such as above 88° C., such as between 70° C. and 95° C., such as between 76° C. and 94° C., such as between 78° C. and 93° C., such as between 80° C. and 92° C., such as between 82° C. and 91° C., such as between 84° C. and 90° C.

In a preferred embodiment, the endogluconase used in a process of the invention or comprised in a composition of the invention is a Glycoside Hydrolase Family 5 endoglucnase or GH5 endoglucanase (see the CAZy database on the world wide web). In an embodiment, the GH5 endoglucanase is from family EG II, such as the Talaromyces leycettanus endoglucanase shown in SEQ ID NO: 7 herein; Penicillium capsulatum endoglucanase shown in SEQ ID NO: 22 herein, and Trichophaea saccata endoglucanase shown in SEQ ID NO: 23 herein. In an embodiment, the endoglucanase is a family GH45 endoglucanase. In an embodiment, the GH45 endoglucanase is from family EG V, such as the Sordaria fimicola shown in SEQ ID NO: 25 herein or the Thielavia terrestris endoglucanase shown in SEQ ID NO: 24 herein.

In an embodiment the endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 7 herein. In an embodiment, the endoglucanase is derived from a strain of the genus Talaromyces, such as a strain of Talaromyces leycettanus.

In an embodiment the endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 22 herein, preferably derived from a strain of the genus Penicillium, such as a strain of Penicillium capsulatum.

In an embodiment the endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 23 herein, preferably derived from a strain of the genus Trichophaea, such as a strain of Trichophaea saccata.

In an embodiment the endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 24 herein, preferably derived from a strain of the genus Thielavia, such as a strain of Thielavia terrestris.

In an embodiment the endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 25 herein, preferably derived from a strain of the genus Sordaria, such as a strain of Sordaria fimicola.

In an embodiment, the endoglucanase is added in liquefaction step i) at a dose from 1-10,000 μg EP (Enzymes Protein)/g DS), such as 10-1,000 μg EP/g DS.

Protease Present and/or Added During Liquefaction

In an embodiment of the invention an optional protease, such as a thermostable protease, may be present and/or added in liquefaction together with a thermostable metal ion inhibition resistant xylanase, preferably having a melting point (DSC) above 80° C., and optionally a thermostable alpha-amylase, endoglucanase, carbohydrate-source generating enzyme, in particular a glucoamylase, optionally a pullulanase and/or optionally a phytase.

Proteases are classified on the basis of their catalytic mechanism into the following groups: Serine proteases (S), Cysteine proteases (C), Aspartic proteases (A), Metallo proteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

In a preferred embodiment the thermostable protease used according to the invention is a “metallo protease” defined as a protease belonging to EC 3.4.24 (metalloendopeptidases); preferably EC 3.4.24.39 (acid metallo proteinases).

To determine whether a given protease is a metallo protease or not, reference is made to the above “Handbook of Proteolytic Enzymes” and the principles indicated therein. Such determination can be carried out for all types of proteases, be it naturally occurring or wild-type proteases; or genetically engineered or synthetic proteases.

Protease activity can be measured using any suitable assay, in which a substrate is employed, that includes peptide bonds relevant for the specificity of the protease in question. Assay-pH and assay-temperature are likewise to be adapted to the protease in question. Examples of assay-pH-values are pH 6, 7, 8, 9, 10, or 11. Examples of assay-temperatures are 30, 35, 37, 40, 45, 50, 55, 60, 65, 70 or 80° C.

Examples of protease substrates are casein, such as Azurine-Crosslinked Casein (AZCL-casein). Two protease assays are described below in the “Materials & Methods”—section, of which the so-called “AZCL-Casein Assay” is the preferred assay.

In an embodiment, the thermostable protease has at least 20%, such as at least 30%, such as at least 40%, such as at least 50%, such as at least 60%, such as at least 70%, such as at least 80%, such as at least 90%, such as at least 95%, such as at least 100% of the protease activity of the JTP196 variant or Protease Pfu (SEQ ID NO: 11 herein) determined by the AZCL-casein assay described in the “Materials & Methods”—section.

There are no limitations on the origin of the thermostable protease used in a process or composition of the invention as long as it fulfills the thermostability properties defined below.

In one embodiment, the protease is of fungal origin.

In a preferred embodiment, the thermostable protease is a variant of a metallo protease as defined above. In an embodiment, the thermostable protease used in a process or composition of the invention is of fungal origin, such as a fungal metallo protease, such as a fungal metallo protease derived from a strain of the genus Thermoascus, preferably a strain of Thermoascus aurantiacus, especially Thermoascus aurantiacus CGMCC No. 0670 (classified as EC 3.4.24.39).

In an embodiment, the thermostable protease is a variant of the mature part of the metallo protease shown in SEQ ID NO: 2 disclosed in WO 2003/048353 or the mature part of SEQ ID NO: 1 in WO 2010/008841 and shown as SEQ ID NO: 6 herein further with mutations selected from below list:
S5*+D79L+S87P+A112P+D142L;
D79L+S87P+A112P+T124V+D142L;
S5*+N26R+D79L+S87P+A112P+D142L;
N26R+T46R+D79L+S87P+A112P+D142L;
T46R+D79L+S87P+T116V+D142L;
D79L+P81R+S87P+A112P+D142L;
A27K+D79L+S87P+A112P+T124V+D142L;
D79L+Y82F+S87P+A112P+T124V+D142L;
D79L+Y82F+S87P+A112P+T124V+D142L;
D79L+S87P+A112P+T124V+A126V+D142L;
D79L+S87P+A112P+D142L;
D79L+Y82F+S87P+A112P+D142L;
S38T+D79L+S87P+A112P+A126V+D142L;
D79L+Y82F+S87P+A112P+A126V+D142L;
A27K+D79L+S87P+A112P+A126V+D142L;
D79L+S87P+N98C+A112P+G135C+D142L;
D79L+S87P+A112P+D142L+T141C+M161C;
S36P+D79L+S87P+A112P+D142L;
A37P+D79L+S87P+A112P+D142L;
S49P+D79L+S87P+A112P+D142L;
S50P+D79L+S87P+A112P+D142L;
D79L+S87P+D104P+A112P+D142L;
D79L+Y82F+S87G+A112P+D142L;
S70V+D79L+Y82F+S87G+Y97W+A112P+D142L;
D79L+Y82F+S87G+Y97W+D104P+A112P+D142L;
S70V+D79L+Y82F+S87G+A112P+D142L;
D79L+Y82F+S87G+D104P+A112P+D142L;
D79L+Y82F+S87G+A112P+A126V+D142L;
Y82F+S87G+S70V+D79L+D104P+A112P+D142L;
Y82F+S87G+D79L+D104P+A112P+A126V+D142L;
A27K+D79L+Y82F+S87G+D104P+A112P+A126V+D142L;
A27K+Y82F+S87G+D104P+A112P+A126V+D142L;
A27K+D79L+Y82F+D104P+A112P+A126V+D142L;
A27K+Y82F+D104P+A112P+A126V+D142L;
A27K+D79L+S87P+A112P+D142L;
D79L+S87P+D142L.

In a preferred embodiment, the thermostable protease is a variant of the mature metallo protease disclosed as the mature part of SEQ ID NO: 2 disclosed in WO 2003/048353 or the mature part of SEQ ID NO: 1 in WO 2010/008841 or SEQ ID NO: 6 herein with the following mutations: D79L+S87P+A112P+D142L;

### D79L+S87P+D142L; or

### A27K+D79L+Y82F+S87G+D104P+A112P+A126V+D142L.

In an embodiment the protease variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 2 disclosed in WO 2003/048353 or the mature part of SEQ ID NO: 1 in WO 2010/008841 or SEQ ID NO: 6 herein.

The thermostable protease may also be derived from any bacterium as long as the protease has the thermostability properties defined according to the invention.

In an embodiment, the thermostable protease is derived from a strain of the bacterium Pyrococcus, such as a strain of Pyrococcus furiosus (pfu protease).

In an embodiment, the protease is one shown as SEQ ID NO: 1 in U.S. Pat. No. 6,358,726-B1 (Takara Shuzo Company) and SEQ ID NO: 11 herein.

In an embodiment, the thermostable protease is one disclosed in SEQ ID NO: 11 herein or a protease having at least 80% identity, such as at least 85%, such as at least 90%, such as at least 95%, such as at least 96%, such as at least 97%, such as at least 98%, such as at least 99% identity to SEQ ID NO: 1 in U.S. Pat. No. 6,358,726-B1 or SEQ ID NO: 11 herein. The Pyroccus furiosus protease can be purchased from Takara Bio, Japan.

The Pyrococcus furiosus protease is a thermostable protease according to the invention. The commercial product Pyrococcus furiosus protease (Pfu S) was found (see Example 5) to have a thermostability of 110% (80° C./70° C.) and 103% (90° C./70° C.) at pH 4.5 determined as described in Example 5 of US-2018-0371505 (which is hereby incorporated by reference for its description of assays for determining thermostability, especially as described in Example 5).

In one embodiment, a thermostable protease has a thermostability value of more than 20% determined as Relative Activity at 80° C./70° C. determined as described in Example 2 of US-2018-0371505 (which is hereby incorporated by reference for its description of assays for determining thermostability, especially as described in Example 2).

In an embodiment, the protease has a thermostability of more than 30%, more than 40%, more than 50%, more than 60%, more than 70%, more than 80%, more than 90%, more than 100%, such as more than 105%, such as more than 110%, such as more than 115%, such as more than 120% determined as Relative Activity at 80° C./70° C.

In an embodiment protease has a thermostability of between 20 and 50%, such as between 20 and 40%, such as 20 and 30% determined as Relative Activity at 80° C./70° C.

In an embodiment, the protease has a thermostability between 50 and 115%, such as between 50 and 70%, such as between 50 and 60%, such as between 100 and 120%, such as between 105 and 115% determined as Relative Activity at 80° C./70° C.

In an embodiment, the protease has a thermostability value of more than 10% determined as Relative Activity at 85° C./70° C. determined as described in Example 2 of US-2018-0371505 (which is hereby incorporated by reference for its description of assays for determining thermostability, especially as described in Example 2).

In an embodiment, the protease has a thermostability of more than 10%, such as more than 12%, more than 14%, more than 16%, more than 18%, more than 20%, more than 30%, more than 40%, more that 50%, more than 60%, more than 70%, more than 80%, more than 90%, more than 100%, more than 110% determined as Relative Activity at 85° C./70° C.

In an embodiment, the protease has a thermostability of between 10 and 50%, such as between 10 and 30%, such as between 10 and 25% determined as Relative Activity at 85° C./70° C.

In an embodiment, the protease has more than 20%, more than 30%, more than 40%, more than 50%, more than 60%, more than 70%, more than 80%, more than 90% determined as Remaining Activity at 80° C.; and/or

In an embodiment, the protease has more than 20%, more than 30%, more than 40%, more than 50%, more than 60%, more than 70%, more than 80%, more than 90% determined as Remaining Activity at 84° C.

Determination of “Relative Activity” and “Remaining Activity” is done as described in Example 2 of US-2018-0371505 (which is hereby incorporated by reference for its description of assays for determining thermostability, especially as described in Example 2).

In an embodiment the protease is derived from a strain of Thermobifida, such as the Thermobifida cellulosytica protease shown in SEQ ID NO: 26 herein, or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 26.

In an embodiment the protease is derived from a strain of Thermobifida, such as the Thermobifida fusca protease shown in SEQ ID NO: 27 herein (referred to as SEQ ID NO: 8 in WO2018/118815 A1, which is incorporated herein by reference in its entirety), or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 27.

In an embodiment the protease is derived from a strain of Thermobifida, such as the Thermobifida halotolerans protease shown in SEQ ID NO: 28 herein (referred to as SEQ ID NO: 10 in WO2018/118815 A1, which is incorporated herein by reference in its entirety), or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 28.

In an embodiment the protease is derived from a strain of Thermococcus, such as the Thermococcus nautili protease shown in SEQ ID NO: 29 herein (referred to as SEQ ID NO: 3 in WO2018/169780A1, which is incorporated herein by reference in its entirety), or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 29.

In an embodiment, the protease may have a themostability for above 90, such as above 100 at 85° C. as determined using the Zein-BCA assay as disclosed in the Materials & Methods section below.

In an embodiment, the protease has a themostability above 60%, such as above 90%, such as above 100%, such as above 110% at 85° C. as determined using the Zein-BCA assay.

In an embodiment protease has a themostability between 60-120, such as between 70-120%, such as between 80-120%, such as between 90-120%, such as between 100-120%, such as 110-120% at 85° C. as determined using the Zein-BCA assay.

In an embodiment, the thermostable protease has at least 20%, such as at least 30%, such as at least 40%, such as at least 50%, such as at least 60%, such as at least 70%, such as at least 80%, such as at least 90%, such as at least 95%, such as at least 100% of the activity of the JTP196 protease variant or Protease Pfu determined by the AZCL-casein assay described in the “Materials & Methods”—section.

Carbohydrate-Source Generating Enzyme Present and/or Added During Liquefaction

According to the invention an optional carbohydrate-source generating enzyme, in particular a glucoamylase, preferably a thermostable glucoamylase, may be present and/or added in liquefaction together with a thermostable metal-ion inhibition resistant xylanase, preferably having a Melting Point (DSC) above 80° C., and an optional alpha-amylase, endoglucanase having a Melting Point (DSC) above 70° C., thermostable protease, and an optional a pullulanase and/or optional phytase.

The term “carbohydrate-source generating enzyme” includes any enzymes generating fermentable sugars. A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrates may be converted directly or indirectly to the desired fermentation product, preferably ethanol. According to the invention a mixture of carbohydrate-source generating enzymes may be used. Specific examples include glucoamylase (being glucose generators), beta-amylase and maltogenic amylase (being maltose generators).

In a preferred embodiment, the carbohydrate-source generating enzyme is thermostable. The carbohydrate-source generating enzyme, in particular thermostable glucoamylase, may be added together with or separately from the alpha-amylase and the thermostable protease. In an embodiment, the carbohydrate-source generating enzyme, preferably a thermostable glucoamylase, has a Relative Activity heat stability at 85° C. of at least 20%, at least 30%, preferably at least 35% determined as described in Example 4 (heat stability) of US-2018-0371505 (which is hereby incorporated by reference for its description of assays for determining heat stability, especially as described in Example 4).

In an embodiment, the carbohydrate-source generating enzyme is a glucoamylase having a relative activity pH optimum at pH 5.0 of at least 90%, preferably at least 95%, preferably at least 97%, such as 100% determined as described in Example 4 (pH optimum).

In an embodiment, the carbohydrate-source generating enzyme is a glucoamylase having a pH stability at pH 5.0 of at least at least 80%, at least 85%, at least 90% determined as described in Example 4 (pH stability).

In a specific and preferred embodiment, the carbohydrate-source generating enzyme is a thermostable glucoamylase, preferably of fungal origin, preferably a filamentous fungi, such as from a strain of the genus Penicillium, especially a strain of Penicillium oxalicum, in particular the Penicillium oxalicum glucoamylase disclosed as SEQ ID NO: 2 in WO 2011/127802 (which is hereby incorporated by reference) and shown in SEQ ID NO: 12 herein.

In an embodiment, the thermostable glucoamylase has at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99% or 100% identity to the mature polypeptide shown in SEQ ID NO: 2 in WO 2011/127802 or SEQ ID NOs: 14 herein.

In an embodiment, the carbohydrate-source generating enzyme, in particular thermostable glucoamylase, is the Penicillium oxalicum glucoamylase shown in SEQ ID NO: 12 herein.

In a preferred embodiment, the carbohydrate-source generating enzyme is a variant of the Penicillium oxalicum glucoamylase disclosed as SEQ ID NO: 2 in WO 2011/127802 and shown in SEQ ID NO: 14 herein, having a K79V substitution (referred to as “PE001”) (using the mature sequence shown in SEQ ID NO: 12 for numbering). The K79V glucoamylase variant has reduced sensitivity to protease degradation relative to the parent as disclosed in WO 2013/036526 (which is hereby incorporated by reference).

Contemplated Penicillium oxalicum glucoamylase variants are disclosed in WO 2013/053801 (which is hereby incorporated by reference).

In an embodiment, these variants have reduced sensitivity to protease degradation.

In an embodiment, these variant have improved thermostability compared to the parent.

More specifically, in an embodiment the glucoamylase has a K79V substitution (using SEQ ID NO: 12 herein for numbering), corresponding to the PE001 variant, and further comprises at least one of the following substitutions or combination of substitutions:

T65A; Q327F; E501V; Y504T; Y504*; T65A+Q327F; T65A+E501V; T65A+Y504T; T65A+Y504*; Q327F+E501V; Q327F+Y504T; Q327F+Y504*; E501V+Y504T; E501V+Y504*; T65A+Q327F+E501V; T65A+Q327F+Y504T; T65A+E501V+Y504T; Q327F+E501V+Y504T; T65A+Q327F+Y504*; T65A+E501V+Y504*; Q327F+E501V+Y504*; T65A+Q327F+E501V+Y504T; T65A+Q327F+E501V+Y504*; E501V+Y504T; T65A+K161S; T65A+Q405T; T65A+Q327W; T65A+Q327F; T65A+Q327Y; P11F+T65A+Q327F; R1K+D3W+K5Q+G7V+N8S+T10K+P11S+T65A+Q327F; P2N+P4S+P11F+T65A+Q327F; P11F+D26C+K330+T65A+Q327F; P2N+P4S+P11F+T65A+Q327W+E501V+Y504T; R1E+D3N+P4G+G6R+G7A+N8A+T10D+P11D+T65A+Q327F; P11F+T65A+Q327W; P2N+P4S+P11F+T65A+Q327F+E501V+Y504T; P11F+T65A+Q327W+E501V+Y504T; T65A+Q327F+E501V+Y504T; T65A+S105P+Q327W; T65A+S105P+Q327F; T65A+Q327W+S364P; T65A+Q327F+S364P; T65A+S103N+Q327F; P2N+P4S+P11F+K34Y+T65A+Q327F; P2N+P4S+P11F+T65A+Q327F+D445N+V447S; P2N+P4S+P11F+T65A+I172V+Q327F; P2N+P4S+P11F+T65A+Q327F+N502*; P2N+P4S+P11F+T65A+Q327F+N502T+P563S+K571E; P2N+P4S+P11F+R31S+K33V+T65A+Q327F+N564D+K571S; P2N+P4S+P11F+T65A+Q327F+S377T; P2N+P4S+P11F+T65A+V325T+Q327W; P2N+P4S+P11F+T65A+Q327F+D445N+V447S+E501V+Y504T; P2N+P4S+P11F+T65A+I172V+Q327F+E501V+Y504T; P2N+P4S+P11F+T65A+Q327F+S377T+E501V+Y504T; P2N+P4S+P11F+D26N+K34Y+T65A+Q327F; P2N+P4S+P11F+T65A+Q327F+I375A+E501V+Y504T; P2N+P4S+P11F+T65A+K218A+K221D+Q327F+E501V+Y504T; P2N+P4S+P11F+T65A+S103N+Q327F+E501V+Y504T; P2N+P4S+T10D+T65A+Q327F+E501V+Y504T; P2N+P4S+F12Y+T65A+Q327F+E501V+Y504T; K5A+P11F+T65A+Q327F+E501V+Y504T; P2N+P4S+T10E+E18N+T65A+Q327F+E501V+Y504T; P2N+T10E+E18N+T65A+Q327F+E501V+Y504T; P2N+P4S+P11F+T65A+Q327F+E501V+Y504T+T568N; P2N+P4S+P11F+T65A+Q327F+E501V+Y504T+K524T+G526A; P2N+P4S+P11F+K34Y+T65A+Q327F+D445N+V447S+E501V+Y504T; P2N+P4S+P11F+R31S+K33V+T65A+Q327F+D445N+V447S+E501V+Y504T; P2N+P4S+P11F+D26N+K34Y+T65A+Q327F+E501V+Y504T; P2N+P4S+P11F+T65A+F80*+Q327F+E501V+Y504T; P2N+P4S+P11F+T65A+K112S+Q327F+E501V+Y504T; P2N+P4S+P11F+T65A+Q327F+E501V+Y504T+T516P+K524T+G526A; P2N+P4S+P11F+T65A+Q327F+E501V+N502T+Y504*; P2N+P4S+P11F+T65A+Q327F+E501V+Y504T; P2N+P4S+P11F+T65A+S103N+Q327F+E501V+Y504T; K5A+P11F+T65A+Q327F+E501V+Y504T; P2N+P4S+P11F+T65A+Q327F+E501V+Y504T+T516P+K524T+G526A; P2N+P4S+P11F+T65A+V79A+Q327F+E501V+Y504T; P2N+P4S+P11F+T65A+V79G+Q327F+E501V+Y504T; P2N+P4S+P11F+T65A+V791+Q327F+E501V+Y504T; P2N+P4S+P11F+T65A+V79L+Q327F+E501V+Y504T; P2N+P4S+P11F+T65A+V79S+Q327F+E501V+Y504T; P2N+P4S+P11F+T65A+L72V+Q327F+E501V+Y504T; S255N+Q327F+E501V+Y504T; P2N+P4S+P11F+T65A+E74N+Q327F+E501V+Y504T; P2N+P4S+P11F+T65A+G220N+Q327F+E501V+Y504T; P2N+P4S+P11F+T65A+Y245N+Q327F+E501V+Y504T; P2N+P4S+P11F+T65A+Q253N+Q327F+E501V+Y504T; P2N+P4S+P11F+T65A+D279N+Q327F+E501V+Y504T; P2N+P4S+P11F+T65A+Q327F+S359N+E501V+Y504T; P2N+P4S+P11F+T65A+Q327F+D370N+E501V+Y504T; P2N+P4S+P11F+T65A+Q327F+V460S+E501V+Y504T; P2N+P4S+P11F+T65A+Q327F+V460T+P468T+E501V+Y504T; P2N+P4S+P11F+T65A+Q327F+T463N+E501V+Y504T; P2N+P4S+P11F+T65A+Q327F+S465N+E501V+Y504T; or P2N+P4S+P11F+T65A+Q327F+T477N+E501V+Y504T.

In a preferred embodiment, the Penicillium oxalicum glucoamylase variant has a K79V substitution using SEQ ID NO: 12 herein for numbering (PE001 variant), and further comprises one of the following mutations: P11F+T65A+Q327F; P2N+P4S+P11F+T65A+Q327F; P11F+D26C+K33C+T65A+Q327F; P2N+P4S+P11F+T65A+Q327W+E501V+Y504T; P2N+P4S+P11F+T65A+Q327F+E501V+Y504T; or P11F+T65A+Q327W+E501V+Y504T.

In an embodiment the glucoamylase variant, such as Penicillium oxalicum glucoamylase variant has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature polypeptide of SEQ ID NO: 12 herein.

The carbohydrate-source generating enzyme, in particular glycoamylase, may be added in amounts from 0.1-100 micrograms EP/g DS, such as 0.5-50 micrograms EP/g DS, such as 1-25 micrograms EP/g DS, such as 2-12 micrograms EP/g DS.

Pullulanase Present and/or Added During Liquefaction

Optionally a pullulanase may be present and/or added during liquefaction step i) together with a metal ion inhibition resistant xylanase, preferably having a melting point (DSC) above 80° C. As mentioned above a thermostable alpha-amylase, protease, carbohydrate-source generating enzyme, preferably a thermostable glucoamylase, may also optionally be present and/or added during liquefaction step i).

The pullulanase may be present and/or added during liquefaction step i) and/or saccharification step ii) or simultaneous saccharification and fermentation.

Pullulanases (E.C. 3.2.1.41, pullulan 6-glucano-hydrolase), are debranching enzymes characterized by their ability to hydrolyze the alpha-1,6-glycosidic bonds in, for example, amylopectin and pullulan.

Contemplated pullulanases according to the present invention include the pullulanases from Bacillus amyloderamificans disclosed in U.S. Pat. No. 4,560,651 (hereby incorporated by reference), the pullulanase disclosed as SEQ ID NO: 2 in WO 01/151620 (hereby incorporated by reference), the Bacillus deramificans disclosed as SEQ ID NO: 4 in WO 01/151620 (hereby incorporated by reference), and the pullulanase from Bacillus acidopullulyticus disclosed as SEQ ID NO: 6 in WO 01/151620 (hereby incorporated by reference) and also described in FEMS Mic. Let. (1994) 115, 97-106.

Additional pullulanases contemplated according to the present invention included the pullulanases from Pyrococcus woesei, specifically from Pyrococcus woesei DSM No. 3773 disclosed in WO 92/02614.

In an embodiment, the pullulanase is a family GH57 pullulanase. In an embodiment, the pullulanase includes an X47 domain as disclosed in WO 2011/087836 (which are hereby incorporated by reference). More specifically the pullulanase may be derived from a strain of the genus Thermococcus, including Thermococcus litoralis and Thermococcus hydrothermalis, such as the Thermococcus hydrothermalis pullulanase shown WO 2011/087836 truncated at the X4 site right after the X47 domain. The pullulanase may also be a hybrid of the Thermococcus litoralis and Thermococcus hydrothermalis pullulanases or a T. hydrothermalis/T. litoralis hybrid enzyme with truncation site X4 disclosed in WO 2011/087836 (which is hereby incorporated by reference).

In another embodiment, the pullulanase is one comprising an X46 domain disclosed in WO 2011/076123 (Novozymes).

The pullulanase may according to the invention be added in an effective amount which include the preferred amount of about 0.0001-10 mg enzyme protein per gram DS, preferably 0.0001-0.10 mg enzyme protein per gram DS, more preferably 0.0001-0.010 mg enzyme protein per gram DS. Pullulanase activity may be determined as NPUN. An Assay for determination of NPUN is described in the “Materials & Methods”—section below.

Suitable commercially available pullulanase products include PROMOZYME 400L, PROMOZYME™ D2 (Novozymes A/S, Denmark), OPTIMAX L-300 (Genencor Int., USA), and AMANO 8 (Amano, Japan).

Phytase Present and/or Added During Liquefaction

Optionally a phytase may be present and/or added in liquefaction in combination with a metal ion inhibition resistant xylanase, preferably having a melting point (DSC) above 80° C. As mentioned above a thermostable alpha-amylase, protease, carbohydrate-source generating enzyme, preferably a thermostable glucoamylase, may also optionally be present and/or added during liquefaction step i).

A phytase used according to the invention may be any enzyme capable of effecting the liberation of inorganic phosphate from phytic acid (myo-inositol hexakisphosphate) or from any salt thereof (phytates). Phytases can be classified according to their specificity in the initial hydrolysis step, viz. according to which phosphate-ester group is hydrolyzed first. The phytase to be used in the invention may have any specificity, e.g., be a 3-phytase (EC 3.1.3.8), a 6-phytase (EC 3.1.3.26) or a 5-phytase (no EC number). In an embodiment, the phytase has a temperature optimum above 50° C., such as in the range from 50-90° C.

The phytase may be derived from plants or microorganisms, such as bacteria or fungi, e.g., yeast or filamentous fungi.

A plant phytase may be from wheat-bran, maize, soy bean or lily pollen. Suitable plant phytases are described in Thomlinson et al, Biochemistry, 1 (1962), 166-171; Barrientos et al, Plant. Physiol., 106 (1994), 1489-1495; WO 98/05785; WO 98/20139.

A bacterial phytase may be from genus Bacillus, Citrobacter, Hafnia, Pseudomonas, Buttiauxella or Escherichia, specifically the species Bacillus subtilis, Citrobacter braakii, Citrobacter freundii, Hafnia alvei, Buttiauxella gaviniae, Buttiauxella agrestis, Buttiauxella noackies and E. coli. Suitable bacterial phytases are described in Paver and Jagannathan, 1982, Journal of Bacteriology 151:1102-1108; Cosgrove, 1970, Australian Journal of Biological Sciences 23:1207-1220; Greiner et al, Arch. Biochem. Biophys., 303, 107-113, 1993; WO 1997/33976; WO 1997/48812, WO 1998/06856, WO 1998/028408, WO 2004/085638, WO 2006/037327, WO 2006/038062, WO 2006/063588, WO 2008/092901, WO 2008/116878, and WO 2010/034835.

A yeast phytase may be derived from genus Saccharomyces or Schwanniomyces, specifically species Saccharomyces cerevisiae or Schwanniomyces occidentalis. The former enzyme has been described as a Suitable yeast phytases are described in Nayini et al, 1984, Lebensmittel Wissenschaft and Technologie 17:24-26; Wodzinski et al, Adv. Appl. Microbiol., 42, 263-303; AU-A-24840/95.

Phytases from filamentous fungi may be derived from the fungal phylum of Ascomycota (ascomycetes) or the phylum Basidiomycota, e.g., the genus Aspergillus, Thermomyces (also called Humicola), Myceliophthora, Manascus, Penicillium, Peniophora, Agrocybe, Paxillus, or Trametes, specifically the species Aspergillus terreus, Aspergillus niger, Aspergillus niger var. awamori, Aspergillus ficuum, Aspergillus fumigatus, Aspergillus oryzae, T. lanuginosus (also known as H. lanuginosa), Myceliophthora thermophila, Peniophora lycii, Agrocybe pediades, Manascus anka, Paxillus involtus, or Trametes pubescens. Suitable fungal phytases are described in Yamada et al., 1986, Agric. Biol. Chem. 322:1275-1282; Piddington et al., 1993, Gene 133:55-62; EP 684,313; EP 0 420 358; EP 0 684 313; WO 1998/28408; WO 1998/28409; JP 7-67635; WO 1998/44125; WO 1997/38096; WO 1998/13480.

In a preferred embodiment the phytase is derived from Buttiauxella, such as Buttiauxella gaviniae, Buttiauxella agrestis, or Buttiauxella noackies, such as the ones disclosed as SEQ ID NO: 2, SEQ ID NO: 4 and SEQ ID NO: 6, respectively, in WO 2008/092901 (hereby incorporated by reference).

In a preferred embodiment the phytase is derived from Citrobacter, such as Citrobacter braakii, such as one disclosed in WO 2006/037328 (hereby incorporated by reference).

Modified phytases or phytase variants are obtainable by methods known in the art, in particular by the methods disclosed in EP 897010; EP 897985; WO 99/49022; WO 99/48330, WO 2003/066847, WO 2007/112739, WO 2009/129489, and WO 2010/034835.

Commercially available phytase containing products include BIO-FEED PHYTASE™, PHYTASE NOVO™ CT or L (all from Novozymes), LIQMAX (DuPont) or RONOZYME™ NP, RONOZYME® HiPhos, RONOZYME® P5000 (CT), NATUPHOS™ NG 5000 (from DSM).

Carbohydrate-Source Generating Enzyme Present and/or Added During Saccharification and/or Fermentation

According to the invention a carbohydrate-source generating enzyme, preferably a glucoamylase, is present and/or added during saccharification and/or fermentation.

In a preferred embodiment the carbohydrate-source generating enzyme is a glucoamylase, of fungal origin, preferably from a stain of Aspergillus, preferably A. niger, A. awamori, or A. oryzae; or a strain of Trichoderma, preferably T. reesei; or a strain of Talaromyces, preferably T. emersonii,

### Glucoamylase

According to the invention the glucoamylase present and/or added in saccharification and/or fermentation may be derived from any suitable source, e.g., derived from a microorganism or a plant. Preferred glucoamylases are of fungal or bacterial origin, selected from the group consisting of Aspergillus glucoamylases, in particular Aspergillus niger G1 or G2 glucoamylase (Boel et al. (1984), EMBO J. 3 (5), p. 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1199-1204.

Other glucoamylases include Athelia rolfsii (previously denoted Corticium rolfsii) glucoamylase (see U.S. Pat. No. 4,727,026 and (Nagasaka et al. (1998) “Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (US patent no. Re. 32,153), Talaromyces duponti, Talaromyces thermophilus (U.S. Pat. No. 4,587,215). In a preferred embodiment the glucoamylase used during saccharification and/or fermentation is the Talaromyces emersonii glucoamylase disclosed in WO 99/28448.

Bacterial glucoamylases contemplated include glucoamylases from the genus Clostridium, in particular C. thermoamylolyticum (EP 135,138), and C. thermohydrosulfuricum (WO 86/01831).

Contemplated fungal glucoamylases include Trametes cingulata, Pachykytospora papyracea; and Leucopaxillus giganteus all disclosed in WO 2006/069289; and Peniophora rufomarginata disclosed in WO2007/124285; or a mixture thereof. Also hybrid glucoamylase are contemplated according to the invention. Examples include the hybrid glucoamylases disclosed in WO 2005/045018. Specific examples include the hybrid glucoamylase disclosed in Table 1 and 4 of Example 1 (which hybrids are hereby incorporated by reference).

In an embodiment the glucoamylase is derived from a strain of the genus Pycnoporus, in particular a strain of Pycnoporus as described in WO 2011/066576 (SEQ ID NOs 2, 4 or 6), or from a strain of the genus Gloephyllum, in particular a strain of Gloephyllum as described in WO 2011/068803 (SEQ ID NO: 2, 4, 6, 8, 10, 12, 14 or 16) or a strain of the genus Nigrofomes, in particular a strain of Nigrofomes sp. disclosed in WO 2012/064351 (SEQ ID NO: 2) (all references hereby incorporated by reference). Contemplated are also glucoamylases which exhibit a high identity to any of the above-mentioned glucoamylases, i.e., at least 70%, at least 75%, at least 80%, at least 85%, at least 90%, at least 95%, at least 96%, at least 97%, at least 98%, at least 99%, such as 100% identity to any one of the mature parts of the enzyme sequences mentioned above.

Glucoamylases may in an embodiment be added to the saccharification and/or fermentation in an amount of 0.0001-20 AGU/g DS, preferably 0.001-10 AGU/g DS, especially between 0.01-5 AGU/g DS, such as 0.1-2 AGU/g DS.

Commercially available compositions comprising glucoamylase include AMG 200L; AMG 300 L; SANT™ SUPER, SANT™ EXTRA L, SPIRIZYME™ PLUS, SPIRIZYME™ FUEL, SPIRIZYME™ B4U, SPIRIZYME™ ULTRA, SPIRIZYME™ EXCEL, SPIRIZYME™ ACHIEVE and AMG™ E (from Novozymes A/S); OPTIDEX™ 300, GC480, GC417 (from Genencor Int.); AMIGASE™ and AMIGASE™ PLUS (from DSM); G-ZYME™ G900, G-ZYME™ and G990 ZR (from Danisco US).

### Maltogenic Amylase

The carbohydrate-source generating enzyme present and/or added during saccharification and/or fermentation may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic amylase from Bacillus stearothermophilus strain NCIB 11837 is commercially available from Novozymes A/S. Maltogenic alpha-amylases are described in U.S. Pat. Nos. 4,598,048, 4,604,355 and 6,162,628, which are hereby incorporated by reference. The maltogenic amylase may in a preferred embodiment be added in an amount of 0.05-5 mg total protein/gram DS or 0.05-5 MANU/g DS.

Cellulase or Cellulolytic Enzyme Composition Present and/or Added During Saccharification and/or Fermentation or SSF

In a preferred embodiment of the invention a cellulase or cellulolytic enzyme composition is present and/or added in saccharification in step ii) and/or fermentation in step iii) or SSF.

The cellulase or cellulolytic enzyme composition may comprise one or more cellulolytic enzymes. The cellulase or cellulolytic enzyme composition may be of any origin. In a preferred embodiment the cellulase or cellulolytic enzyme composition comprises cellulolytic enzymes of fungal origin. In an embodiment the cellulase or cellulolytic enzyme composition is derived from a strain of Trichoderma, such as Trichoderma reesei; or a strain of Humicola, such as Humicola insolens; or a strain of Chrysosporium, such as Chrysosporium lucknowense; or a strain of Penicillium, such as Penicillium decumbens. In a preferred embodiment the cellulolytic enzyme composition is derived from a strain of Trichoderma reesei. The cellulase may be a beta-glucosidase, a cellobiohydrolase, and an endoglucanase or a combination thereof. The cellulolytic enzyme composition may comprise a beta-glucosidase, a cellobiohydrolase, and an endoglucanase.

In an embodiment the cellulase or cellulolytic enzyme composition comprising one or more polypeptides selected from the group consisting of:—beta-glucosidase; —cellobiohydrolase I; —cellobiohydrolase II; or a mixture thereof.

In an embodiment, the cellulase or cellulolytic enzyme composition comprising one or more polypeptides selected from the group consisting of: —beta-glucosidase; —cellobiohydrolase; and —endoglucanase; or a mixture thereof.

In an embodiment the cellulase or cellulolytic enzyme composition comprising one or more polypeptides selected from the group consisting of: —beta-glucosidase; —cellobiohydrolase I; and —endoglucanase; or a mixture thereof.

In a preferred embodiment the cellulase or cellulolytic enzyme composition further comprises a GH61 polypeptide having cellulolytic enhancing activity. Cellulolytic enhancing activity is defined and determined as described in WO 2011/041397 (incorporated by reference).

The term “GH61 polypeptide having cellulolytic enhancing activity” means a GH61 polypeptide that enhances the hydrolysis of a cellulosic material by enzymes having cellulolytic activity. For purposes of the present invention, cellulolytic enhancing activity may be determined by measuring the increase in reducing sugars or the increase of the total of cellobiose and glucose from hydrolysis of a cellulosic material by cellulolytic enzyme under the following conditions: 1-50 mg of total protein/g of cellulose in PCS (Pretreated Corn Stover), wherein total protein is comprised of 50-99.5% w/w cellulolytic enzyme protein and 0.5-50% w/w protein of a GH61 polypeptide having cellulolytic enhancing activity for 1-7 days at 50° C. compared to a control hydrolysis with equal total protein loading without cellulolytic enhancing activity (1-50 mg of cellulolytic protein/g of cellulose in PCS). In a preferred aspect, a mixture of CELLUCLAST™ 1.5 L (Novozymes A/S, Bagsværd, Denmark) in the presence of 2-3% of total protein weight Aspergillus oryzae beta-glucosidase (recombinantly produced in Aspergillus oryzae according to WO 02/095014) or 2-3% of total protein weight Aspergillus fumigatus beta-glucosidase (recombinantly produced in Aspergillus oryzae as described in WO 2002/095014) of cellulase protein loading is used as the source of the cellulolytic activity.

The cellulolytic enzyme composition may comprise a beta-glucosidase, preferably one derived from a strain of the genus Aspergillus, such as Aspergillus oryzae, such as the one disclosed in WO 2002/095014 or the fusion protein having beta-glucosidase activity disclosed in WO 2008/057637 (see SEQ ID NOs: 74 or 76), or Aspergillus fumigatus, such as one disclosed in SEQ ID NO: 2 in WO 2005/047499 or SEQ ID NO: 16 herein; or an Aspergillus fumigatus beta-glucosidase variant disclosed in WO 2012/044915; or a strain of the genus a strain Penicillium, such as a strain of the Penicillium brasilianum disclosed in WO 2007/019442, or a strain of the genus Trichoderma, such as a strain of Trichoderma reesei.

In an embodiment the beta-glucosidase is from a strain of Aspergillus, such as a strain of Aspergillus fumigatus, such as Aspergillus fumigatus beta-glucosidase (SEQ ID NO: 16 herein), or a variant thereof, which variant comprises one or more substitutions selected from the group consisting of L89M, G91L, F100D, I140V, I186V, S283G, N456E, and F512Y; such as a variant thereof with the following substitutions:
F100D+S283G+N456E+F512Y; —L89M+G91L+I186V+I140V;
I186V+L89M+G91L+I140V+F100D+S283G+N456E+F512Y (using SEQ ID NO: 16 herein for numbering).

The parent beta-glucosidase may have at least 60% identity, such as at least 70%, such as at least 80%, such as at least 90%, such as at least 95%, such as at least 96%, such as at least 97%, such as at least 98%, such as at least 99%, such as 100% identity to the mature polypeptide of SEQ ID NO: 16 herein.

In case the beta-glucosidase is a beta-glucosidase variant it may have at least 60% identity, such as at least 70%, such as at least 80%, such as at least 90%, such as at least 95%, such as at least 96%, such as at least 97%, such as at least 98%, such as at least 99%, but less than 100% identity to the mature polypeptide of SEQ ID NO: 16 herein.

In case the cellulolytic enzyme composition may comprise a GH61 polypeptide, it may be one derived from the genus Thermoascus, such as a strain of Thermoascus aurantiacus, such as the one described in WO 2005/074656 as SEQ ID NO: 6 or SEQ ID NO: 18 herein; or one derived from the genus Thielavia, such as a strain of Thielavia terrestris, such as the one described in WO 2005/074647 as SEQ ID NO: 7 and SEQ ID NO: 8; or one derived from a strain of Aspergillus, such as a strain of Aspergillus fumigatus, such as the one described in WO 2010/138754 as SEQ ID NO: 1 and SEQ ID NO: 2; or one derived from a strain derived from Penicillium, such as a strain of Penicillium emersonii, such as the one disclosed in WO 2011/041397 as SEQ ID NO: 6 or SEQ ID NO: 19 herein.

In a preferred embodiment the GH61 polypeptide, such as one derived from a strain of Penicillium, preferably a strain of Penicillium emersonii, is selected from the group consisting of:

(i) a GH61 polypeptide comprising the mature polypeptide of SEQ ID NO: 19 herein;
(ii) a GH61 polypeptide comprising an amino acid sequence having at least 60%, such as at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the mature polypeptide of SEQ ID NO: 19 herein.

In an embodiment the cellulolytic enzyme composition comprises a cellobiohydrolase I (CBH I), such as one derived from a strain of the genus Aspergillus, such as a strain of Aspergillus fumigatus, such as the Cel7a CBH I disclosed in SEQ ID NO: 6 in WO 2011/057140 or SEQ ID NO: 20 herein, or a strain of the genus Trichoderma, such as a strain of Trichoderma reesei.

In a preferred embodiment the cellobiohydrolase I, such as one derived from a strain of Aspergillus, preferably a strain of Aspergillus fumigatus, is selected from the group consisting of:

(i) a cellobiohydrolase I comprising the mature polypeptide of SEQ ID NO: 20 herein;
(ii) a cellobiohydrolase I comprising an amino acid sequence having at least 60%, such as at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the mature polypeptide of SEQ ID NO: 20 herein.

In an embodiment the cellulolytic enzyme composition comprises a cellobiohydrolase II (CBH II), such as one derived from a strain of the genus Aspergillus, such as a strain of Aspergillus fumigatus; such as the one disclosed as SEQ ID NO: 21 herein or a strain of the genus Trichoderma, such as Trichoderma reesei, or a strain of the genus Thielavia, such as a strain of Thielavia terrestris, such as cellobiohydrolase II CEL6A from Thielavia terrestris.

In a preferred embodiment cellobiohydrolase II, such as one derived from a strain of Aspergillus, preferably a strain of Aspergillus fumigatus, is selected from the group consisting of:

(i) a cellobiohydrolase II comprising the mature polypeptide of SEQ ID NO: 21 herein;
(ii) a cellobiohydrolase II comprising an amino acid sequence having at least 60%, such as at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the mature polypeptide of SEQ ID NO: 21 herein.

In an embodiment the cellulolytic enzyme composition comprises a GH61 polypeptide having cellulolytic enhancing activity and a beta-glucosidase.

In an embodiment the cellulolytic enzyme composition comprises a GH61 polypeptide having cellulolytic enhancing activity derived from a strain of Penicillium, such as a strain of Penicillium emersonii, such as the one disclosed as SEQ ID NO: 2 in WO 2011/041397 or SEQ ID NO: 19 herein, and a beta-glucosidase.

In an embodiment the cellulolytic enzyme composition comprises a GH61 polypeptide having cellulolytic enhancing activity, a beta-glucosidase, and a CBH I.

In an embodiment the cellulolytic enzyme composition comprises a GH61 polypeptide having cellulolytic enhancing activity derived from a strain of Penicillium, such as a strain of Penicillium emersonii, such as the one disclosed as SEQ ID NO: 2 in WO 2011/041397 or SEQ ID NO: 19 herein, a beta-glucosidase, and a CBHI.

In an embodiment the cellulolytic enzyme composition comprises a GH61 polypeptide having cellulolytic enhancing activity, a beta-glucosidase, a CBHI, and a CBHII.

In an embodiment the cellulolytic enzyme composition comprises a GH61 polypeptide having cellulolytic enhancing activity derived from a strain of Penicillium, such as a strain of Penicillium emersonii, such as the one disclosed as SEQ ID NO: 2 in WO 2011/041397 or SEQ ID NO: 19 herein, a beta-glucosidase, a CBHI, and a CBHII.

In an embodiment the cellulolytic enzyme composition is a Trichoderma reesei cellulolytic enzyme composition, further comprising Thermoascus aurantiacus GH61A polypeptide (SEQ ID NO: 2 in WO 2005/074656 or SEQ ID NO: 18 herein), and Aspergillus oryzae beta-glucosidase fusion protein (WO 2008/057637).

In an embodiment the cellulolytic enzyme composition is a Trichoderma reesei cellulolytic enzyme composition, further comprising Thermoascus aurantiacus GH61A polypeptide having cellulolytic enhancing activity (SEQ ID NO: 2 in WO 2005/074656 or SEQ ID NO: 18 herein) and Aspergillus fumigatus beta-glucosidase (SEQ ID NO: 2 of WO 2005/047499 or SEQ ID NO: 16 herein).

In an embodiment the cellulolytic enzyme composition is a Trichoderma reesei cellulolytic enzyme composition further comprising Penicillium emersonii GH61A polypeptide disclosed as SEQ ID NO: 2 in WO 2011/041397 or SEQ ID NO: 19 herein, and Aspergillus fumigatus beta-glucosidase disclosed as SEQ ID NO: 2 in WO 2005/047499 or SEQ ID NO: 16 herein, or a variant thereof, which variant has one of, preferably all of the following substitutions: F100D, S283G, N456E, F512Y.

In an embodiment the cellulolytic enzyme composition comprises one or more of the following components: (i) an Aspergillus fumigatus cellobiohydrolase I; (ii) an Aspergillus fumigatus cellobiohydrolase II; and (iii) an Aspergillus fumigatus beta-glucosidase or variant thereof.

In an embodiment the cellulolytic enzyme composition comprises one or more of the following components: (i) an Aspergillus fumigatus cellobiohydrolase I; (ii) an Aspergillus fumigatus beta-glucosidase or variant thereof; and (iii) a Trichoderma reesei endoglucanase I.

In an embodiment the Aspergillus fumigatus beta-glucosidase (SEQ ID NO: 16 herein), comprises one or more substitutions selected from the group consisting of L89M, G91L, F100D, I140V, I186V, S283G, N456E, and F512Y; such as a variant thereof, with the following substitutions: —F100D+S283G+N456E+F512Y; —L89M+G91L+I186V+I140V; or —I186V+L89M+G91L+I140V+F100D+S283G+N456E+F512Y.

In an embodiment the cellulolytic enzyme composition further comprises the Penicillium sp. GH61 polypeptide shown in SEQ ID NO: 19 herein; or a GH61 polypeptide comprising an amino acid sequence having at least 60%, such as at least 70%, e.g., at least 75%, at least 80%, at least 85%, at least 90%, at least 91%, at least 92%, at least 93%, at least 94%, at least 95%, at least 96%, at least 97%, at least 98%, or at least 99%, such as 100% identity to the mature polypeptide of SEQ ID NO: 19 herein.

In an embodiment the cellulolytic enzyme composition further comprises the Trichoderma reesei polypeptide shown in SEQ ID NO: 17 herein; or a polypeptide comprising an amino acid sequence having at least 60%, such as at least 70%, e.g., at least 75%, at least 80%, at least 85%, at least 90%, at least 91%, at least 92%, at least 93%, at least 94%, at least 95%, at least 96%, at least 97%, at least 98%, or at least 99%, such as 100% identity to the mature polypeptide of SEQ ID NO: 17 herein.

In an embodiment the cellulolytic enzyme composition comprising the following components: (i) Aspergillus fumigatus cellobiohydrolase 1 shown in SEQ ID NO: 20 herein; (ii) Aspergillus fumigatus cellobiohydrolase 11 shown in SEQ ID NO: 21 herein; (iii) a variant of Aspergillus fumigatus beta-glucosidase shown in SEQ ID NO: 16 with the following substitutions: F100D+S283G+N456E+F512Y; and (iv) Penicillium sp. GH61 polypeptide shown in SEQ ID NO: 19 herein.

In an embodiment, the cellulolytic enzyme composition comprising the following components: (i) Aspergillus fumigatus cellobiohydrolase I shown in SEQ ID NO: 20 herein; (ii) a variant of Aspergillus fumigatus beta-glucosidase shown in SEQ ID NO: 16 with the following substitutions: F100D+S283G+N456E+F512Y; and (iii) Trichoderma reesei endoglucanase I shown in SEQ ID NO: 17 herein.

In an embodiment, the cellulolytic enzyme composition is derived from Trichoderma reesei and further comprises: (i) Aspergillus fumigatus cellobiohydrolase I shown in SEQ ID NO: 20 herein; (ii) a variant of Aspergillus fumigatus beta-glucosidase shown in SEQ ID NO: 16 with the following substitutions: F100D+S283G+N456E+F512Y; and (iii) Trichoderma reesei endoglucanase I shown in SEQ ID NO: 17 herein.

In an embodiment, cellulolytic enzyme composition is dosed (i.e. during saccharification in step ii) and/or fermentation in step iii) or SSF) from 0.0001-3 mg EP/g DS, preferably 0.0005-2 mg EP/g DS, preferably 0.001-1 mg/g DS, more preferred from 0.005-0.5 mg EP/g DS, even more preferred 0.01-0.1 mg EP/g DS.

### Examples of Preferred Processes of the Invention

In a preferred embodiment, the invention relates to a process for producing fermentation products from starch-containing material comprising the steps of:

i) liquefying the starch-containing material at a pH in the range between from above 4.0-6.5 at a temperature in the range from 70-100° C. using:
an alpha-amylase derived from Bacillus stearothermophilus;
a xylanase that is resistant to inhibition by metal ions in the liquefying starch-containing material, and further having a Melting Point (DSC) above 80° C.
an optional endoglucanase having a Melting Point (DSC) above 70° C.;

ii) saccharifying using a glucoamylase enzyme;

iii) fermenting using a fermenting organism.

In a preferred embodiment the process of the invention comprises the steps of:

i) liquefying the starch-containing material at a pH in the range between from above 4.5-6.2 at a temperature above the initial gelatinization temperature using:
an alpha-amylase, preferably derived from Bacillus stearothermophilus, having a T½ (min) at pH 4.5, 85° C., 0.12 mM CaCl₂ of at least 10;
a xylanase that is resistant to inhibition by metal ions in the liquefying starch-containing material, and further having a Melting Point (DSC) above 80° C.;
an optional endoglucanase having a Melting Point (DSC) above 70° C.;
   ii) saccharifying using a glucoamylase enzyme;
   iii) fermenting using a fermenting organism.

In a preferred embodiment the process of the invention comprises the steps of:

i) liquefying the starch-containing material at a pH in the range between from above 4.0-6.5 at a temperature between 70-100° C.:
a bacterial alpha-amylase, preferably derived from Bacillus stearothermophilus, having a T½ (min) at pH 4.5, 85° C., 0.12 mM CaCl₂ of at least 10;
a xylanase that is resistant to inhibition by metal ions in the liquefying starch-containing material, and further having a Melting Point (DSC) above 80° C.;—an optional endoglucanase having a Melting Point (DSC), between 70° C. and 95° C.;
optionally a protease, preferably derived from Pyrococcus furiosus or Thermoascus aurantiacus, having a thermostability value of more than 20% determined as Relative Activity at 80° C./70° C.;
   ii) saccharifying using a glucoamylase enzyme;
   iii) fermenting using a fermenting organism.

In a preferred embodiment the process of the invention comprises the steps of:

i) liquefying the starch-containing material at a pH in the range between from above 4.0-6.5 at a temperature above the initial gelatinization temperature using:
an alpha-amylase shown in SEQ ID NO: 5 having a double deletion in positions R179+G180 or I181+G182, and optional substitution N193F; and optionally further one of the following set of substitutions:
E129V+K177L+R179E;
V59A+Q89R+E129V+K177L+R179E+H208Y+K220P+N224L+Q254S;
V59A+E129V+K177L+R179S+Q254S;
E129V+K177L+R179E+K220P+N224L+S242Q+Q254S;
V59A+Q89R+E129V+K177L+R179E+Q254S+M284V (using SEQ ID NO: 5 herein for numbering);
a xylanase that is resistant to inhibition by metal ions in the liquefying starch-containing material, having a Melting Point (DSC) above 80° C.;
such as an xylanase having at least 60%, such as at least 70%, such as at least 75% identity, preferably at least 80%, at least 81%, at least 82%, at least 83%, at least 84%, more preferably at least 85%, at least 86%, at least 87%, at least 88%, at least 89%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NOs: 2, 3, and 4 herein;
an optional endoglucanase having a Melting Point (DSC), between 70° C. and 95° C.; such as an endoglucanase having at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% to the mature part of the any of the polypeptides shown in SEQ ID NOs: 7, 22, 23, 24 or 25 herein;
optionally a protease having a thermostability value of more than 20% determined as Relative Activity at 80° C./70° C. derived from Pyrococcus furiosus and/or Thermoascus aurantiacus;
optionally a Penicillium oxalicum glucoamylase in SEQ ID NO: 12 herein, preferably having substitutions selected from the group of:
K79V;
K79V+P11F+T65A+Q327F; or
K79V+P2N+P4S+P11F+T65A+Q327F; (using SEQ ID NO: 12 for numbering);
   ii) saccharifying using a glucoamylase enzyme;
   iii) fermenting using a fermenting organism.

In a preferred embodiment the process of the invention comprises the steps of:

i) liquefying the starch-containing material at a pH in the range between from above 4.0-6.5 at a temperature between 70-100° C. using:
an alpha-amylase derived from Bacillus stearothermophilus having a double deletion in positions I181+G182, and optional substitution N193F; and optionally further one of the following set of substitutions:
I181*+G182*+N193F+E129V+K177L+R179E;
N193F+V59A+Q89R+E129V+K177L+R179E+H208Y+K220P+N224L+Q254S
N193F+V59A+Q89R+E129V+K177L+R179E+Q254S+M284V;
N193F+V59A+E129V+K177L+R179E+Q254S+M284V;
N193F+E129V+K177L+R179E+K220P+N224L+S242Q+Q254S;
V59A+E129V+K177L+R179E+Q254S+M284V+V212T+Y268G+N293Y+T297N;
V59A+E129V+K177L+R179E+Q254S+M284V+V212T+Y268G+N293Y+T297N+S173N+E188P+H208Y+S242Y+K279I;
V59A+E129V+K177L+R179S+Q254S+M284V+V212T+Y268G+N293Y+T297N+A184Q+E188P+T191N
V59A+E129V+K177L+R179S+Q254S+M284V+V212T+Y268G+N293Y+T297N+A184Q+E188P+T191N+S242Y+K279I;
V59A+E129V+K177L+R179S+Q254S+M284V+V212T+Y268G+N293Y+T297N+A184Q+E188P+T191N+N193F+S242Y+K279I;
V59A+E129V+K177L+R179E+Q254S+M284V+V212T+Y268G+N293Y+T297N+E188P+K279W;
V59A+E129V+K177L+R179E+Q254S+M284V+V212T+Y268G+N293Y+T297N+W115D+D117Q+T133P;
V59A+Q89R+E129V+K177L+R179E+H208Y+K220P+N224L+Q254S; or
V59A+Q89R+E129V+K177L+R179E+Q254S+M284V (using SEQ ID NO: 5 herein for numbering);
a xylanase that is resistant to inhibition by metal ions in the liquefying starch-containing material, having a Melting Point (DSC) above 80° C.;
such as an xylanase having at least 60%, such as at least 70%, such as at least 75% identity, preferably at least 80%, at least 81%, at least 82%, at least 83%, at least 84%, more preferably at least 85%, at least 86%, at least 87%, at least 88%, at least 89%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NOs: 2, 3, and 4 herein;
an optional endoglucanase having a Melting Point (DSC), between 70° C. and 95° C.; preferably having at least 90% identity to the mature part of the polypeptide of SEQ ID NO: 7 herein;
a optional protease having a thermostability value of more than 20% determined as Relative Activity at 80° C./70° C. derived from Pyrococcus furiosus and/or Thermoascus aurantiacus; and
optionally a Penicillium oxalicum glucoamylase in SEQ ID NO: 12 herein, preferably having substitutions selected from the group of:
K79V; or
K79V+P11F+T65A+Q327F; or
K79V+P2N+P4S+P11F+T65A+Q327F (using SEQ ID NO: 12 herein for numbering);
   ii) saccharifying using a glucoamylase enzyme;
   iii) fermenting using a fermenting organism.

In a preferred embodiment the process of the invention comprises the steps of:

i) liquefying the starch-containing material at a pH in the range between from above 4.0-6.5 at a temperature between 70-100° C. using:
an alpha-amylase derived from Bacillus stearothermophilus having a double deletion in positions I181+G182, and optional substitution N193F; and optionally further one of the following set of substitutions:
V59A+Q89R+E129V+K177L+R179S+H208Y+K220P+N224L+Q254S; or
V59A+Q89R+E129V+K177L+R179S+Q254S+M284V (using SEQ ID NO: 5 herein for numbering);
a xylanase that is resistant to inhibition by metal ions in the liquefying starch-containing material, and further having a Melting Point (DSC) above 80° C.;
such as an xylanase having at least 60%, such as at least 70%, such as at least 75% identity, preferably at least 80%, at least 81%, at least 82%, at least 83%, at least 84%, more preferably at least 85%, at least 86%, at least 87%, at least 88%, at least 89%, at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NOs: 2, 3, and 4 herein;
an optional endoglucanase having a Melting Point (DSC), between 70° C. and 95° C.; preferably having at least 90% identity to the mature part of the polypeptide of SEQ ID NO: 7 herein;
a optional protease having a thermostability value of more than 20% determined as Relative Activity at 80° C./70° C. derived from Pyrococcus furiosus and/or Thermoascus aurantiacus; and
optionally a Penicillium oxalicum glucoamylase in SEQ ID NO: 12 herein, preferably having substitutions selected from the group of:
K79V; or
K79V+P11F+T65A+Q327F; or
K79V+P2N+P4S+P11F+T65A+Q327F (using SEQ ID NO: 12 herein for numbering);
   ii) saccharifying using a glucoamylase enzyme;
   iii) fermenting using a fermenting organism.

In a preferred embodiment the process of the invention comprises the steps of:

i) liquefying the starch-containing material at a pH in the range between from above 4.0-6.5 at a temperature between 70-100° C. using:
an alpha-amylase derived from Bacillus stearothermophilus having a double deletion in positions I181+G182, and optional substitution N193F; and optionally further one of the following set of substitutions:
I181*+G182*+N193F+E129V+K177L+R179E;
N193F+V59A+Q89R+E129V+K177L+R179E+H208Y+K220P+N224L+Q254S
N193F+V59A+Q89R+E129V+K177L+R179E+Q254S+M284V;
N193F+V59A+E129V+K177L+R179E+Q254S+M284V;
N193F+E129V+K177L+R179E+K220P+N224L+5242Q+Q254S;
V59A+E129V+K177L+R179E+Q254S+M284V+V212T+Y268G+N293Y+T297N;
V59A+E129V+K177L+R179E+Q254S+M284V+V212T+Y268G+N293Y+T297N+S173N+E188P+H208Y+S242Y+K279I;
V59A+E129V+K177L+R179S+Q254S+M284V+V212T+Y268G+N293Y+T297N+A184Q+E188P+T191N
V59A+E129V+K177L+R179S+Q254S+M284V+V212T+Y268G+N293Y+T297N+A184Q+E188P+T191N+S242Y+K279I;
V59A+E129V+K177L+R179S+Q254S+M284V+V212T+Y268G+N293Y+T297N+A184Q+E188P+T191N+N193F+S242Y+K279I;
V59A+E129V+K177L+R179E+Q254S+M284V+V212T+Y268G+N293Y+T297N+E188P+K279W;
V59A+E129V+K177L+R179E+Q254S+M284V+V212T+Y268G+N293Y+T297N+W115D+D117Q+T133P;
E129V+K177L+R179E;
V59A+Q89R+E129V+K177L+R179E+H208Y+K220P+N224L+Q254S;
V59A+Q89R+E129V+K177L+R179E+Q254S+M284V;
E129V+K177L+R179E+K220P+N224L+S242Q+Q254S (using SEQ ID NO: 5 herein for numbering);
a xylanase that is resistant to inhibition by metal ions in the liquefying starch-containing material, having a Melting Point (DSC) above 80° C.;
an optional endoglucanase having a Melting Point (DSC), between 70° C. and 95° C.; preferably having at least 90% identity to the mature part of the polypeptide of SEQ ID NO: 7 herein;
a protease having a thermostability value of more than 20% determined as Relative Activity at 80° C./70° C. derived from Pyrococcus furiosus;
a Penicillium oxalicum glucoamylase in SEQ ID NO: 12 herein, preferably having substitutions selected from the group of:
K79V;
K79V+P11F+T65A+Q327F; or
K79V+P2N+P4S+P11F+T65A+Q327F; or
K79V+P11F+D26C+K33C+T65A+Q327F; or
K79V+P2N+P4S+P11F+T65A+Q327W+E501V+Y504T; or
K79V+P2N+P4S+P11F+T65A+Q327F+E501V+Y504T; or
K79V+P11F+T65A+Q327W+E501V+Y504T (using SEQ ID NO: 12 herein for numbering);
   ii) saccharifying using a glucoamylase enzyme;
   iii) fermenting using a fermenting organism.

In a preferred embodiment, the process of the invention comprises the steps of:

i) liquefying the starch-containing material at a pH in the range between from above 4.0-6.5 at a temperature between 80-95° C. using:
an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations I181*+G182*+N193F+V59A+Q89R+E129V+K177L+R179E+Q254S+M284V;
a thermostable metal ion inhibition resistant xylanase, more preferably a thermostable xylanase having at least 80%, at least 85%, at least 90%, or at least 95%, at least 96%, at least 97%, at least 98%, at least 99%, or 100% identity to the amino acid sequence of the mature polypeptide of SEQ ID NO: 2; and
a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 11;

ii) saccharifying using a glucoamylase enzyme;

iii) fermenting using a fermenting organism.

In a preferred embodiment, the process of the invention comprises the steps of:

i) liquefying the starch-containing material at a pH in the range between from above 4.0-6.5 at a temperature between 80-95° C. using:
an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+5242Y+K279I;
a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 2; and
a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 11;

ii) saccharifying using a glucoamylase enzyme;

iii) fermenting using a fermenting organism.

In a preferred embodiment, the process of the invention comprises the steps of:

i) liquefying the starch-containing material at a pH in the range between from above 4.0-6.5 at a temperature between 80-95° C. using:
an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+5242Y+K279I;
a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 3; and
a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 11;

ii) saccharifying using a glucoamylase enzyme; and

iii) fermenting using a fermenting organism.

In a preferred embodiment, the process of the invention comprises the steps of:

i) liquefying the starch-containing material at a pH in the range between from above 4.0-6.5 at a temperature between 80-95° C. using:
an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+5242Y+K279I;
a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 3; and

a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 11;

ii) saccharifying using a glucoamylase enzyme; and

iii) fermenting using a fermenting organism.

In a preferred embodiment, the process of the invention comprises the steps of:

i) liquefying the starch-containing material at a pH in the range between from above 4.0-6.5 at a temperature between 80-95° C. using:
an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+5242Y+K279I;
a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 4; and
a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 11;

ii) saccharifying using a glucoamylase enzyme;

iii) fermenting using a fermenting organism.

In a preferred embodiment, the process of the invention comprises the steps of:

i) liquefying the starch-containing material at a pH in the range between from above 4.0-6.5 at a temperature between 80-95° C. using:
an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+5242Y+K279I;
a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 4; and
a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 11;

ii) saccharifying using a glucoamylase enzyme; and

iii) fermenting using a fermenting organism.

In a preferred embodiment, the process of the invention comprises the steps of:

i) liquefying the starch-containing material at a pH in the range between from above 4.0-6.5 at a temperature between 80-95° C. using:
an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+5242Y+K279I;
a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 2; and
a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 26, SEQ ID NO: 27, SEQ ID NO: 28, or SEQ ID NO: 29;

ii) saccharifying using a glucoamylase enzyme; and

iii) fermenting using a fermenting organism.

In a preferred embodiment, the process of the invention comprises the steps of:

i) liquefying the starch-containing material at a pH in the range between from above 4.0-6.5 at a temperature between 80-95° C. using:
an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+5242Y+K279I;
a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 2; and
a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 26, SEQ ID NO: 27, SEQ ID NO: 28, or SEQ ID NO: 29;

ii) saccharifying using a glucoamylase enzyme; and

iii) fermenting using a fermenting organism.

In a preferred embodiment, the process of the invention comprises the steps of:

i) liquefying the starch-containing material at a pH in the range between from above 4.0-6.5 at a temperature between 80-95° C. using:
an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+5242Y+K279I;
a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 3; and
a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 26, SEQ ID NO: 27, SEQ ID NO: 28, or SEQ ID NO: 29;

ii) saccharifying using a glucoamylase enzyme; and

iii) fermenting using a fermenting organism.

In a preferred embodiment, the process of the invention comprises the steps of:

i) liquefying the starch-containing material at a pH in the range between from above 4.0-6.5 at a temperature between 80-95° C. using:
an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+5242Y+K279I;
a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 3; and

a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 26, SEQ ID NO: 27, SEQ ID NO: 28, or SEQ ID NO: 29;

ii) saccharifying using a glucoamylase enzyme; and

iii) fermenting using a fermenting organism.

In a preferred embodiment, the process of the invention comprises the steps of:

i) liquefying the starch-containing material at a pH in the range between from above 4.0-6.5 at a temperature between 80-95° C. using:
an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+5242Y+K279I;
a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 4; and
a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 26, SEQ ID NO: 27, SEQ ID NO: 28, or SEQ ID NO: 29;

ii) saccharifying using a glucoamylase enzyme; and

iii) fermenting using a fermenting organism.

In a preferred embodiment, the process of the invention comprises the steps of:

i) liquefying the starch-containing material at a pH in the range between from above 4.0-6.5 at a temperature between 80-95° C. using:
an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+5242Y+K279I;
a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 4; and
a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 26, SEQ ID NO: 27, SEQ ID NO: 28, or SEQ ID NO: 29.

ii) saccharifying using a glucoamylase enzyme; and

iii) fermenting using a fermenting organism.

In a preferred embodiment, a cellulase or cellulolytic enzyme composition is present and/or added during fermentation or simultaneous saccharification and fermentation.

In a preferred embodiment, a cellulase or cellulolytic enzyme composition derived from Trichoderma reesei is present and/or added during fermentation or simultaneous saccharification and fermentation (SSF).

In a preferred embodiment, a cellulase or cellulolytic enzyme composition and a glucoamylase are present and/or added during fermentation or simultaneous saccharification and fermentation.

In an embodiment, the cellulase or cellulolytic enzyme composition is derived from Trichoderma reesei, Humicola insolens, Chrysosporium lucknowense or Penicillium decumbens.

### A Composition of the Invention

A composition of the invention comprises an alpha-amylase, such as a thermostable alpha-amylase, and a xylanase that is resistant to inhibition by metal ions in liquefying starch-containing material, having a Melting Point (DSC) above 80° C.; an optional endoglucanase having a Melting Point (DSC) above 70° C.; an optional protease, such as a thermostable protease. The composition may also further comprise a carbohydrate-source generating enzyme, in particular a glucoamylase, optionally a pullulanase and optionally a phytase too. Therefore, in this aspect the invention relates to composition comprising:
an alpha-amylase;
a xylanase that is resistant to inhibition by metal ions in liquefying starch-containing material, and further having a Melting Point (DSC) above 80° C.
an optional endoglucanase having a Melting Point (DSC) above 70° C.;
optionally a protease;
optionally a carbohydrate-source generating enzyme.
   Alpha-amylase: The alpha-amylase may be any alpha-amylase. In a preferred embodiment the alpha-amylase is a bacterial alpha-amylases, such as alpha-amylases derived from the genus Bacillus, such as Bacillus stearomthermphilus, preferably the one shown in SEQ ID NO: 5 herein.

The alpha-amylase may be a thermostable alpha-amylase. The thermostable alpha-amylase may have a T½ (min) at pH 4.5, 85° C., 0.12 mM CaCl₂) of at least 10, such as at least 15, such as at least 20, such as at least 25, such as at least 30, such as at least 40, such as at least 50, such as at least 60, such as between 10-70, such as between 15-70, such as between 20-70, such as between 25-70, such as between 30-70, such as between 40-70, such as between 50-70, such as between 60-70.

In an embodiment the alpha-amylase is selected from the group of Bacillus stearomthermphilus alpha-amylase variants, in particular truncated to be 491 amino acids long, such as from 480 to 495 amino acids long, with mutations selected from the group of:

### I181*+G182*;

### I181*+G182*+N193F;

preferably

### I181*+G182*+E129V+K177L+R179E;

### I181*+G182*+N193F+E129V+K177L+R179E;

### I181*+G182*+N193F+V59A+Q89R+E129V+K177L+R179E+H208Y+K220P+N224L+Q254S;

### I181*+G182*+N193F+E129V+K177L+R179E;

### N193F+V59A+Q89R+E129V+K177L+R179E+H208Y+K220P+N224L+Q254S

### N193F+V59A+Q89R+E129V+K177L+R179E+Q254S+M284V;

### N193F+V59A+E129V+K177L+R179E+Q254S+M284V;

### N193F+E129V+K177L+R179E+K220P+N224L+S242Q+Q254S;

### V59A+E129V+K177L+R179E+Q254S+M284V+V212T+Y268G+N293Y+T297N;

### V59A+E129V+K177L+R179E+Q254S+M284V+V212T+Y268G+N293Y+T297N+S173N+E188P+H208Y+S242Y+K279I;

### V59A+E129V+K177L+R179S+Q254S+M284V+V212T+Y268G+N293Y+T297N+A184Q+E188P+T191N

### V59A+E129V+K177L+R179S+Q254S+M284V+V212T+Y268G+N293Y+T297N+A184Q+E188P+T191N+S242Y+K279I;

### V59A+E129V+K177L+R179S+Q254S+M284V+V212T+Y268G+N293Y+T297N+A184Q+E188P+T191N+N193F+S242Y+K279I;

### V59A+E129V+K177L+R179E+Q254S+M284V+V212T+Y268G+N293Y+T297N+E188P+K279W;

### V59A+E129V+K177L+R179E+Q254S+M284V+V212T+Y268G+N293Y+T297N+W115D+D117Q+T133P;

### I181*+G182*+N193F+V59A Q89R+E129V+K177L+R179E+Q254S+M284V; and

I181*+G182*+N193F+E129V+K177L+R179E+K220P+N224L+S242Q+Q254S (using SEQ ID NO: 5 herein for numbering).

It should be understood that these alpha-amylases are only specific examples. Any alpha-amylase disclosed above in the “Alpha-Amylase Present and/or Added During Liquefaction”—section above may be used as the alpha-amylase component in a composition of the invention.

Endoglucanase: According to the invention an optional endoglucanase component may be comprised in the composition. It may be any endoglucanase having a Melting Point (DSC) above 70° C., such as above 75° C., in particular above 80° C., such as between 70° C. and 95° C., determined using the “Differential Scanning Calorimetry (DSC) Assay” described in the “Materials & Methods”—section below.

In an embodiment the endoglucanase has a Melting Point (DSC) above 72° C., such as above 74° C., such as above 76° C., such as above 78° C., such as above 80° C., such as above 82° C., such as above 84° C., such as above 86° C., such as above 88° C., such as between 70° C. and 95° C., such as between 76° C. and 94° C., such as between 78° C. and 93° C., such as between 80° C. and 92° C., such as between 82° C. and 91° C., such as between 84° C. and 90° C.

In a preferred embodiment the endogluconase used in a process of the invention comprised in a composition of the invention is a Glycoside Hydrolase Family 5 endoglucnase or GH5 endoglucanase (see the CAZy database on the “www.cazy.org” webpage. In an embodiment the GH5 endoglocianase is from family EG II, such as the Talaromyces leycettanus endoglucanase shown in SEQ ID NO: 7 herein; Penicillium capsulatum endoglucanase show in SEQ ID NO: 22 herein, and Trichophaea saccata endoglucanase shown in SEQ ID NO: 23 herein.

In an embodiment the endoglucanase is a family GH45 endoglucanase. In an embodiment the GH45 endoglocianase is from family EG V, such as the Sordaria fimicola shown in SEQ ID NO: 25 herein or Thielavia terrestris endoglucnase shown in SEQ ID NO: 24 herein.

In an embodiment the endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 7 herein. In an embodiment the endoglucanase is derived from a strain of the genus Talaromyces, such as a strain of Talaromyces leycettanus.

In an embodiment the endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 22 herein, preferably derived from a strain of the genus Penicillium, such as a strain of Penicillium capsulatum.

In an embodiment the endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 23 herein, preferably derived from a strain of the genus Trichophaea, such as a strain of Trichophaea saccata.

In an embodiment the endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 24 herein, preferably derived from a strain of the genus Thielavia, such as a strain of Thielavia terrestris.

In an embodiment the endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 25 herein, preferably derived from a strain of the genus Sordaria, such as a strain of Sordaria fimicola.

It should be understood that these endoglucanases are only specific examples. Any endoglucanase disclosed above in the “Thermostable Endoglucanase Present and/or Added During Liquefaction”—section above may be used as the optional endoglucoanase component in a composition of the invention.

In an especially preferred embodiment the endoglucanase (EG) has at least 90% identity to the mature part of the polypeptide of SEQ ID NO: 7 herein derived from a strain of Talaromyces leycettanus having a Melting Point (DSC) above 80° C.

Protease: A composition of the invention may optionally comprise a protease, such as a thermostable protease. There is no limitation on the origin of the protease component as long as it fulfills the thermostability properties defined herein.

In an embodiment the protease is of fungal origin. In an embodiment the protease is a metallo protease. In an embodiment the protease is derived from Thermoascus aurantiacus shown in SEQ ID NO: 6 herein.

In a preferred embodiment the protease is a variant of the Thermoascus aurantiacus protease mentioned above having a thermostability value of more than 20% determined as Relative Activity at 80° C./70° C. determined as described in Example 2 of US-2018-0371505 0371505 (which is hereby incorporated by reference for its description of assays for determining thermostability, especially as described in Example 2).

In a specific preferred embodiment the protease is a variant of the metallo protease derived from Thermoascus aurantiacus disclosed as the mature part of SEQ ID NO: 2 disclosed in WO 2003/048353 or the mature part of SEQ ID NO: 1 in WO 2010/008841 or SEQ ID NO: 6 herein with mutations selected from the group of:
D79L+S87P+A112P+D142L;
D79L+S87P+D142L; and
A27K+D79L+Y82F+S87G+D104P+A112P+A126V+D142L.

In another embodiment the protease is a bacterial protease. In another embodiment the protease is a serine protease. In a preferred embodiment the serine protease is derived from a strain of Pyrococcus furiosus, such as the one shown in SEQ ID NO: 1 in U.S. Pat. No. 6,358,726 or SEQ ID NO: 11 herein, or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 1. In an embodiment the serine protease is derived from a strain of Thermobifida, such as the one shown in SEQ ID NO: 26 herein, or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 26. In an embodiment the protease is derived from a strain of Thermobifida, such as the Thermobifida fusca protease shown in SEQ ID NO: 27 herein (referred to as SEQ ID NO: 8 in WO2018/118815 A1, which is incorporated herein by reference in its entirety), or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 27. In an embodiment the protease is derived from a strain of Thermobifida, such as the Thermobifida halotolerans protease shown in SEQ ID NO: 28 herein (referred to as SEQ ID NO: 10 in WO2018/118815 A1, which is incorporated herein by reference in its entirety), or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 28. In an embodiment the protease is derived from a strain of Thermococcus, such as the Thermococcus nautili protease shown in SEQ ID NO: 29 herein (referred to as SEQ ID NO: 3 in WO2018/169780A1, which is incorporated herein by reference in its entirety), or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 29.

It should be understood that these proteases are only examples. Any protease disclosed above in the “Protease Present and/or Added During Liquefaction” section above may be used as the protease component in a composition of the invention.

Carbohydrate-source generating enzymes: A composition of the invention may optionally further comprise a carbohydrate-source generating enzyme, in particular a glucoamylase, such as a thermostable glucoamylase which has a heat stability at 85° C., pH 5.3, of at least 30%, preferably at least 35%.

Said carbohydrate-source generating enzyme may be a thermostable glucoamylase having a Relative Activity heat stability at 85° C. of at least 20%, at least 30%, preferably at least 35% determined as described in Example 4 (Heat stability).

In an embodiment the carbohydrate-source generating enzyme is a glucoamylase having a relative activity pH optimum at pH 5.0 of at least 90%, preferably at least 95%, preferably at least 97%, such as 100% determined as described in Example 4 (pH optimum).

In an embodiment the carbohydrate-source generating enzyme is a glucoamylase having a pH stability at pH 5.0 of at least at least 80%, at least 85%, at least 90% determined as described in Example 4 (pH stability).

In a preferred embodiment the carbohydrate-source generating enzyme is a thermostable glucoamylase, preferably of fungal origin, preferably a filamentous fungi, such as from a strain of the genus Penicillium, especially a strain of Penicillium oxalicum disclosed as SEQ ID NO: 2 in WO 2011/127802 (which is hereby incorporated by reference), or a variant thereof, and shown in SEQ ID NO: 12 herein.

In an embodiment the glucoamylase, or a variant thereof, may have at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature polypeptide shown in SEQ ID NO: 2 in WO 2011/127802 or SEQ ID NO: 12 herein.

In a specific and preferred embodiment the carbohydrate-source generating enzyme is a variant of the Penicillium oxalicum glucoamylase disclosed as SEQ ID NO: 2 in WO 2011/127802 and shown in SEQ ID NO: 12 herein, having a K79V substitution (using the mature sequence shown in SEQ ID NO: 12 herein for numbering). The K79V glucoamylase variant has reduced sensitivity to protease degradation relative to the parent as disclosed in WO 2013/036526 (which is hereby incorporated by reference).

Examples of suitable thermostable Penicillium oxalicum glucoamylase variants are listed above.

In an embodiment the carbohydrate-source generating enzyme, such as glucoamyase, such as Penicillium oxalicum glucoamylase, has pullulanase side-activity.

It should be understood that these carbohydrate-source generating enzymes, in particular glucoamylases, are only examples. Any carbohydrate-source generating enzyme disclosed above in the “Carbohydrate-source generating enzyme Present and/or Added During Liquefaction” section above may be used as component in a composition of the invention.

In a preferred embodiment the carbohydrate-source generating enzyme is the Penicillium oxalicum glucoamylase shown in SEQ ID NO: 12 herein or a sequence having at least 90% identity thereto further comprising a K79V substitution.

Pullulanase: A composition of the invention may optionally further comprise a pullulanase. The pullulanase may be of any origin.

In an embodiment the pullulanase is of bacterial origin. In an embodiment the pullulanase is derived from a strain of Bacillus sp.

In an embodiment the pullulanase is a family GH57 pullulanase. In a preferred embodiment the pullulanase includes an X47 domain as disclosed in WO 2011/087836 (which is hereby incorporated by reference).

Specifically the pullulanase may be derived from a strain from the genus Thermococcus, including Thermococcus litoralis and Thermococcus hydrothermalis or a hybrid thereof.

The pullulanase may be Thermococcus hydrothermalis pullulanase shown in SEQ ID NO: 9 herein truncated at site X4 or a Thermococcus hydrothermalis/T. litoralis hybrid enzyme (SEQ ID NO: 10 herein) with truncation site X4 as disclosed in WO 2011/087836.

The another embodiment the pullulanase is one comprising an X46 domain disclosed in WO 2011/076123 (Novozymes).

It should be understood that these pullulanases are only specific examples. Any pullulanase disclosed above in the “Pullulanase Present and/or Added During Liquefaction”—section above may be used as the optional pullulanase component in a composition of the invention.

Phytase: A composition of the invention may optionally further comprise a phytase. The phytase may be of any origin.

In an embodiment the phytase is of bacterial origin. In an embodiment the phytase is derived from a strain of from Buttiauxella, such as Buttiauxella gaviniae, such as the one disclosed as SEQ ID NO: 2 (amino acids 1-33 are expected signal peptide) in WO 2008/092901; or Buttiauxella agrestis, such as the one shown as SEQ ID NO: 4 (amino acids −9 to −1 are expected to be a part of the signal peptide) in WO 2008/092901; or Buttiauxella noackies, such as the one shown as SEQ ID NO: 6 in WO 2008/092901.

In another embodiment the phytase is derived from a strain of Citrobacter, such as a strain of Citrobacter braakii, such as ones disclosed as SEQ ID NOs: 2 or 4 in WO 2006/037328 (hereby incorporated by reference).

It should be understood that these phytases are only specific examples. Any phytase disclosed above in the “Phytase Present and/or Added During Liquefaction”—section above may be used as the optional pullulanase component in a composition of the invention.

In a preferred embodiment the phytase is derived from a strain of Buttiauxella.

### Examples of Preferred Embodiments of the Composition of the Invention

In a preferred embodiment the composition of the invention comprises
an alpha-amylase derived from Bacillus stearothermophilus;
a xylanase that is resistant to inhibition by metal ions in liquefying starch-containing material, and further having a Melting Point (DSC) above 80° C.;
an optional endoglucanase having a Melting Point (DSC) above 70° C., such as between 70° C. and 95° C.;
optionally a protease having a thermostability value of more than 20% determined as Relative Activity at 80° C./70° C. derived from Pyrococcus furiosus or Thermoascus auranticus; and
optionally a glucoamylase, such as one derived from Penicillium oxalicum.

In another embodiment the composition of the invention comprises
an alpha-amylase, preferably derived from Bacillus stearothermophilus, having a T½ (min) at pH 4.5, 85° C., 0.12 mM CaCl₂) of at least 10;
a xylanase that is resistant to inhibition by metal ions in liquefying starch-containing material, and further having a Melting Point (DSC) above 80° C.;
an optional endoglucanase having a Melting Point (DSC) between 70° C. and 95° C.;
an optional protease, preferably derived from Pyrococcus furiosus or Thermoascus aurantiacus, having a thermostability value of more than 20% determined as Relative Activity at 80° C./70° C.; and
optionally a glucoamylase, e.g., derived from Penicillium oxalicum.

In another embodiment the composition of the invention comprises
an alpha-amylase derived from Bacillus stearothermophilus having a double deletion in positions I181+G182, and optionally substitution N193F; and optionally further one of the following set of substitutions:
I181*+G182*+E129V+K177L+R179E;
I181*+G182*+N193F+E129V+K177L+R179E;
I181*+G182*+N193F+V59A+Q89R+E129V+K177L+R179E+H208Y+K220P+N224L+Q254S;
I181*+G182*+N193F+E129V+K177L+R179E;
N193F+V59A+Q89R+E129V+K177L+R179E+H208Y+K220P+N224L+Q254S
N193F+V59A+Q89R+E129V+K177L+R179E+Q254S+M284V;
N193F+V59A+E129V+K177L+R179E+Q254S+M284V;
N193F+E129V+K177L+R179E+K220P+N224L+S242Q+Q254S;
V59A+E129V+K177L+R179E+Q254S+M284V+V212T+Y268G+N293Y+T297N;
V59A+E129V+K177L+R179E+Q254S+M284V+V212T+Y268G+N293Y+T297N+S173N+E188P+H208Y+S242Y+K279I;
V59A+E129V+K177L+R179S+Q254S+M284V+V212T+Y268G+N293Y+T297N+A184Q+E188P+T191N
V59A+E129V+K177L+R179S+Q254S+M284V+V212T+Y268G+N293Y+T297N+A184Q+E188P+T191N+S242Y+K279I;
V59A+E129V+K177L+R179S+Q254S+M284V+V212T+Y268G+N293Y+T297N+A184Q+E188P+T191N+N193F+S242Y+K279I;
V59A+E129V+K177L+R179E+Q254S+M284V+V212T+Y268G+N293Y+T297N+E188P+K279W;
V59A+E129V+K177L+R179E+Q254S+M284V+V212T+Y268G+N293Y+T297N+W115D+D117Q+T133P;
I181*+G182*+N193F+V59A Q89R+E129V+K177L+R179E+Q254S+M284V; and
I181*+G182*+N193F+E129V+K177L+R179E+K220P+N224L+S242Q+Q254S (using SEQ ID NO: 5 herein for numbering);
a xylanase that is resistant to inhibition by metal ions in liquefying starch-containing material and further having a Melting Point (DSC) above 80° C.;
an optional endoglucanase having a Melting Point (DSC) above 70° C.;
optionally a protease, preferably derived from Pyrococcus furiosus and/or Thermoascus aurantiacus, having a thermostability value of more than 20% determined as Relative Activity at 80° C./70° C.; and
optionally a Penicillium oxalicum glucoamylase in SEQ ID NO: 12 having substitutions selected from the group of:
K79V;
K79V+P11F+T65A+Q327F; or
K79V+P2N+P4S+P11F+T65A+Q327F; or
K79V+P11F+D26C+K33C+T65A+Q327F; or
K79V+P2N+P4S+P11F+T65A+Q327W+E501V+Y504T; or
K79V+P2N+P4S+P11F+T65A+Q327F+E501V+Y504T; or
K79V+P11F+T65A+Q327W+E501V+Y504T (using SEQ ID NO: 12 for numbering).

In an embodiment the Bacillus stearothermophilus alpha-amylase (SEQ ID NO: 5 herein), or a variant thereof, is the mature alpha-amylase or corresponding mature alpha-amylases having at least 80% identity, at least 90% identity, at least 95% identity at least 96% identity at least 97% identity at least 99% identity to the SEQ ID NO: 5 herein.

In an embodiment the xylanase such as from the genus Thermotoga, has a Melting Point (DSC) above 82° C., such as above 84° C., such as above 86° C., such as above 88° C., such as above 88° C., such as above 90° C., such as above 92° C., such as above 94° C., such as above 96° C., such as above 98° C., such as above 100° C., such as between 80° C. and 110° C., such as between 82° C. and 110° C., such as between 84° C. and 110° C.

Examples of suitable thermostable xylanases, in particular xylanases from the genus Thermotoga, include the xylanase shown in SEQ ID NOs: 2 herein, e.g., derived from a strain of Thermotoga maritima; the xylanase shown in SEQ ID NO: 3 herein, e.g., derived from a strain of Thermotoga neapolitana; the xylanase shown in SEQ ID NO: 5 herein, e.g., derived from a strain of Thermotoga naphthophila; or polypeptides having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity, such as 100% identity to the mature part of any of the polypeptides of SEQ ID NOs: 2, 3, and 4 herein, respectively.

In a preferred embodiment, the composition of the invention comprises—an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations I181*+G182*+N193F+V59A+Q89R+E129V+K177L+R179E+Q254S+M284V; —a thermostable metal ion inhibition resistant xylanase, more preferably a thermostable xylanase having at least 80%, at least 85%, at least 90%, or at least 95%, at least 96%, at least 97%, at least 98%, at least 99%, or 100% identity to the amino acid sequence of the mature polypeptide of SEQ ID NO: 2; and a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 11.

In a preferred embodiment, the composition of the invention comprises —an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+S242Y+K279I; —a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 2; and a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 11.

In a preferred embodiment, the composition of the invention comprises—an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+S242Y+K279I; —a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 2; and a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 11.

In a preferred embodiment, the composition of the invention comprises—an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+S242Y+K279I; —a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 3; and a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 11.

In a preferred embodiment, the composition of the invention comprises—an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+S242Y+K279I; —a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 3; and a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 11.

In a preferred embodiment, the composition of the invention comprises—an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+S242Y+K279I; —a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 4; and a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 11.

In a preferred embodiment, the composition of the invention comprises—an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+S242Y+K279I; —a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 4; and a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 11.

In a preferred embodiment, the composition of the invention comprises—an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+S242Y+K279I; —a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 2; and a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 26, SEQ ID NO: 27, SEQ ID NO: 28, or SEQ ID NO: 29.

In a preferred embodiment, the composition of the invention comprises—an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+S242Y+K279I; —a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 2; and a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 26, SEQ ID NO: 27, SEQ ID NO: 28, or SEQ ID NO: 29.

In a preferred embodiment, the composition of the invention comprises—an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+S242Y+K279I; —a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 3; and a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 26, SEQ ID NO: 27, SEQ ID NO: 28, or SEQ ID NO: 29.

In a preferred embodiment, the composition of the invention comprises—an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+S242Y+K279I; —a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 3; and a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 26, SEQ ID NO: 27, SEQ ID NO: 28, or SEQ ID NO: 29.

In a preferred embodiment, the composition of the invention comprises—an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+S242Y+K279I; —a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 4; and a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 26, SEQ ID NO: 27, SEQ ID NO: 28, or SEQ ID NO: 29.

In a preferred embodiment, the composition of the invention comprises—an alpha-amylase, preferably a thermostable bacterial alpha-amylase, more preferably an alpha-amylase having at least 80%, at least 85%, at least 90%, or at least 95%, at least 96%, at least 97%, at least 98%, at least 99%, or 100% identity to the amino acid sequence of amino acids 1 to 491 of SEQ ID NO: 5 with the mutations—I181*+G182*+V59A+E129V+K177L+R179S+Q254S+M284V+V212T+Y268G+N293Y+T297N+A184Q+E188P+T191N+N193F+S242Y+K279I; —a thermostable metal ion inhibition resistant xylanase, more preferably a thermostable metal ion inhibition resistant xylanase having at least 80%, at least 85%, at least 90%, or at least 95%, at least 96%, at least 97%, at least 98%, at least 99%, or 100% identity to the amino acid sequence of the mature polypeptide of SEQ ID NO: 4; and a thermostable protease having at least 80%, at least 85%, at least 90%, or at least 95%, at least 96%, at least 97%, at least 98%, at least 99%, or 100% identity to the amino acid sequence of the mature polypeptide of SEQ ID NO: 26, SEQ ID NO: 27, SEQ ID NO: 28, or SEQ ID NO: 29.

In an embodiment the optional endoglucoanase has a Melting Point (DSC) above 74° C., such as above 76° C., such as above 78° C., such as above 80° C., such as above 82° C., such as above 84° C., such as above 86° C., such as above 88° C., such as between 70° C. and 95° C., such as between 76° C. and 94° C., such as between 78° C. and 93° C., such as between 80° C. and 92° C., such as between 82° C. and 91° C., such as between 84° C. and 90° C.

In an embodiment the optional endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NOs: 7, 22, 23, 24 or 25 herein.

In an embodiment, the endoglucanase has at least 80% identity to the mature part of the polypeptide of SEQ ID NO: 7 herein.

In an embodiment, the endoglucanase has at least 90% identity to the mature part of the polypeptide of SEQ ID NO: 7 herein having a Melting Point (DSC) above 70° C.

In an embodiment, the Pyrococcus furiosus protease (SEQ ID NO: 11 herein) and/or Thermoascus aurantiacus protease (SEQ ID NO: 6 herein), or a variant thereof, is the mature protease or corresponding mature protease having at least 80% identity, at least 85% identity, at least 90% identity, at least 95% identity at least 96% identity at least 97% identity at least 99% identity to the SEQ ID NO: 6 herein or SEQ ID NO: 11 herein, respectively.

In an embodiment 1 the Penicillium oxalicum glucoamylase (SEQ ID NO: 12 herein), or a variant thereof, is the mature glucoamylase or corresponding mature glucoamylase having at least 80% identity, at least 85% identity, at least 90% identity, at least 95% identity at least 96% identity at least 97% identity at least 99% identity to the SEQ ID NO: 12 herein.

### Further Aspects of the Invention

In a further aspect of the invention it relates to the use of a composition of the invention for liquefying a starch-containing material.

In a further aspect of the invention is relates to methods of producing liquefied starch, comprising liquefying a starch-containing material with a composition of the invention.

In a further aspect of the invention it relates to the use of a xylanase that is resistant to metal ion inhibition for liquefying a starch-containing material.

In a further aspect of the invention it relates to methods of producing liquefied starch, comprising liquefying a starch-containing material with a xylanase that is resistant to metal ion inhibition.

In a further aspect of the invention it relates to use of a composition of the invention for reducing the residual starch in a liquefact.

In a further aspect of the invention it relates to the use of a xylanase that is resistant to metal ion inhibition for reducing the residual starch in a liquefact.

In a further aspect of the invention it relates to use of a composition of the invention for increasing short chain oligosaccharides in a liquefact.

In a further aspect of the invention it relates to the use of a xylanase that is resistant to metal ion inhibition for increasing short chain oligosaccharides in a liquefact

The invention is further summarized in the following paragraphs:

1. A process for producing a fermentation product from a starch-containing material comprising the steps of:
i) liquefying a starch-containing material at a temperature above the initial gelatinization temperature in the presence of thermostable xylanase that is resistance to inhibition by metal ions in the liquefying starch-containing material;
ii) saccharifying using a carbohydrate-source generating enzyme; and
iii) fermenting using a fermenting organism to produce the fermentation product.
2. The process of paragraph 1, wherein the amount of residual starch present at the end of liquefying step i) is decreased compared to the amount of residual starch present at the end of liquefying step i) in the absence of the xylanase or compared to the amount of residual starch at the end of liquefying step i) when using a thermostable xylanase that is not resistant or is less resistant to inhibition by metal ions in the liquefying starch-containing material, such as for example the xylanase of SEQ ID NO: 1.
3. The process of paragraph 1, wherein the amount of short chain oligosaccharides present at the end of liquefying step i) is increased compared to the amount of short chain oligosaccharides at the end of liquefying step i) when using a thermostable xylanase that is not resistant or is less resistant to inhibition by metal ions in the liquefying starch-containing material, such as for example the xylanase of SEQ ID NO: 1.
4. A process for decreasing the amount of residual starch present in a liquefact, comprising:

i) liquefying a starch-containing material with thermostable xylanase that is resistant to inhibition by metal ions in the liquefying starch-containing material to produce a liquefact, wherein the liquefact has a decreased amount of residual starch compared to a liquefact produced without the thermostable xylanase or when using a thermostable xylanase that is not resistant or is less resistant to inhibition by metal ions in the liquefying starch-containing material;

optionally ii) saccharifying using a carbohydrate-source generating enzyme; and

optionally iii) fermenting using a fermenting organism to produce the fermentation product.

5. A process for increasing the amount of short-chain oligosaccharides present in a liquefact, comprising:

i) liquefying a starch-containing material with thermostable xylanase that is resistant to inhibition by metal ions in the liquefying starch-containing material to produce a liquefact, wherein the liquefact has an increased amount of short-chain oligosaccharides compared to a liquefact produced without the thermostable xylanase or when using a thermostable xylanase that is not resistant or is less resistant to inhibition by metal ions in the liquefying starch-containing material;

optionally ii) saccharifying using a carbohydrate-source generating enzyme; and optionally iii) fermenting using a fermenting organism to produce the fermentation product.

6. The process of paragraphs 4 or 5, further comprising ii) saccharifying using a carbohydrate-source generating enzyme; and optionally iii) fermenting using a fermenting organism to produce the fermentation product.
7. The process of any one of paragraphs 1 to 6, wherein the thermostable xylanase is derived from a strain of the genus Thermatoga.
8. The process of any one of paragraphs 1 to 7, wherein the thermostable xylanase has at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity, such as 100% identity to the mature part of the polypeptide of SEQ ID NO: 2 herein, preferably derived from a strain of the genus Thermotoga, such as a strain of Thermotoga maritima.
9. The process any of paragraphs 1 to 8, wherein the thermostable xylanase has at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity, such as 100% identity to the mature part of the polypeptide of SEQ ID NO: 3 herein, preferably derived from a strain of the genus Thermotoga, such as a strain of Thermotoga neopolitana.
10. The process of any of paragraphs 1 to 9, wherein the thermostable xylanase has at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity, such as 100% identity to the mature part of the polypeptide of SEQ ID NO: 4 herein, preferably derived from a strain of the genus Thermotoga, such as a strain of Thermotoga naphthophila.
11. The process of any one of paragraphs 1 to 10, wherein the thermostable xylanase has a Melting Point (DSC) above 82° C., such as above 84° C., such as above 86° C., such as above 88° C., such as above 90° C., such as above 92° C., such as above 94° C., such as above 96° C., such as above 98° C., such as above 100° C., such as between 80° C. and 110° C., such as between 82° C. and 110° C., such as between 84° C. and 110° C.
12. The process of any one of paragraphs 1 to 11, wherein the starch-containing material is corn and the metal ions in the liquefying starch-containing material are Copper, Iron and Zinc ions.
13. The process of any one of paragraphs 1 to 12, wherein resistance to inhibition by metal ions in the liquefying starch-containing material is the retention of at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of the relative activity of the xylanase in the presence of the average concentration of the metal ion in the liquefying starch-containing material.
14. The process of any one of paragraphs 1 to 13, wherein the thermostable xylanase retains at least 80% of its relative activity in the presence of Copper ions in the liquefying starch-containing material.
15. The process of any one of paragraphs 1 to 14, wherein the thermostable xylanase retains at least 70% of its relative activity in the presence of Iron ions in the liquefying starch-containing material.
16. The process of any one of paragraphs 1 to 15, wherein the thermostable xylanase retains at least 95% of its relative activity in the presence of Zinc ions in the liquefying starch-containing material.
17. The process of any one of paragraphs 1 to 16, wherein the average concentration of metal ions present in the liquefying starch-containing material ranges from 0.012 mM to 0.15 mM.
18. The process of any one of paragraphs 1 to 17, wherein the average concentration of Copper ions present in the corn is 0.012 mM.
19. The process of any one of paragraphs 1 to 18, wherein the average concentration of Iron ions present in the corn is 0.15 mM.
20. The process of any one of paragraphs 1 to 19, wherein the average concentration of Zinc ions present in the corn is 0.12 mM.
21. The process of any one of paragraphs 1 to 20, wherein a thermostable alpha-amylase and/or thermostable protease are present in liquefying step i).
22. The process of any one of paragraphs 1 to 21, wherein the thermostable alpha-amylase has at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity, such as 100% identity to the mature part of the polypeptide of SEQ ID NO: 5 herein, preferably derived from a strain of Bacillus, such as Bacillus stearothermophilus.
23. The process of any one of paragraphs 1 to 22, wherein the thermostable protease is selected from the group consisting of:

(i) a protease derived from a strain of Pyrococcus, such as the Pyrococcus furiosus protease shown in SEQ ID NO: 11, or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 11.

(ii) a protease derived from a strain of Thermobifida, such as the Thermobifida cellulosytica protease shown in SEQ ID NO: 26 herein, or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 26, or the Thermobifida fusca protease shown in SEQ ID NO: 27 herein, or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 27, or the Thermobifida halotolerans protease shown in SEQ ID NO: 28, or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 28; and

(iii) a protease derived from a strain of Thermococcus, such as the Thermococcus nautili protease shown in SEQ ID NO: 29 herein, or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 29.

24. The process of any of paragraphs 1 to 23, wherein the pH during liquefying step i) is between 4.0-6.5, such as 4.5-6.2, such as above 4.8-6.0, such as between 5.0-5.8.
25. The process of any one of paragraphs 1 to 24, wherein the temperature during liquefaction is in the range from 70-100° C., such as between 70-95° C., such as between 75-90° C., preferably between 80-90° C., such as around 85° C.
26. The process of any of paragraphs 1 to 25, wherein saccharification and fermentation is carried out sequentially or simultaneously.
27. The process of any of paragraphs 1 to 27, wherein the fermentation product is an alcohol, preferably ethanol, especially fuel ethanol, potable ethanol and/or industrial ethanol.
28. The process of any of paragraphs 1 to 28, wherein the starch-containing starting material is whole grains.
29. The process of any of paragraphs 1 to 28, wherein the starch-containing material is derived from corn, wheat, barley, rye, milo, sago, cassava, manioc, tapioca, sorghum, rice or potatoes.
30. The process of any of paragraphs 1 to 29, wherein the fermenting organism is yeast, preferably a strain of Saccharomyces, especially a strain of Saccharomyces cerevisiae.
31. The process of any of paragraphs 1 to 30, wherein the thermostable xylanase is a GH10 family xylanase that contains the motif YITEMD (SEQ ID NO: 30).

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control. Various references are cited herein, the disclosures of which are incorporated by reference in their entireties. The present invention is further described by the following examples which should not be construed as limiting the scope of the invention.

### Materials & Methods

### Materials:

Alpha-Amylase 369 (AA369): Bacillus stearothermophilus alpha-amylase with the mutations: I181*+G182*+N193F+V59A+Q89R+E129V+K177L+R179E+Q254S+M284V truncated to 491 amino acids (SEQ ID NO: 5 herein).
Alpha-Amylase 2330 (AA2330): Bacillus stearothermophilus alpha-amylase with the mutations: —I181*+G182*+V59A+E129V+K177L+R179S+Q254S+M284V+V212T+Y268G+N293Y+T297N+A184Q+E188P+T191N+N193F+S242Y+K279I truncated to 491 amino acids (SEQ ID NO: 5 herein).
Endoglucanase TL (EG TL): Endoglucoanase GH5 from Talaromyces leycettanus disclosed in WO2013/019780 as SEQ ID NO: 2 and SEQ ID NO: 7 herein. (P23YSQ).
Protease Pfu: Protease derived from Pyrococcus furiosus purchased from Takara Bio (Japan) as Pfu Protease S (activity 10.5 mg/mL) and also shown in SEQ ID NO: 11 herein.
Glucoamylase Po: Mature part of the Penicillium oxalicum glucoamylase disclosed as SEQ ID NO: 2 in WO 2011/127802 and shown in SEQ ID NO: 12 herein.
Glucoamylase PE001: Variant of the Penicillium oxalicum glucoamylase having a K79V substitution using the mature sequence shown in SEQ ID NO: 12 for numbering.
Glucoamylase Po 498 (GA498): Variant of Penicillium oxalicum glucoamylase having the following mutations: K79V+P2N+P4S+P11F+T65A+Q327F (using SEQ ID NO: 12 for numbering).
Glucoamylase SA (GSA): Blend comprising Talaromyces emersonii glucoamylase disclosed as SEQ ID NO: 34 in WO99/28448 or SEQ ID NO: 15 herein, Trametes cingulata glucoamylase disclosed as SEQ ID NO: 2 in WO 06/69289 or SEQ ID NO: 13 herein, and Rhizomucor pusillus alpha-amylase with Aspergillus niger glucoamylase linker and starch binding domain (SBD) disclosed in SEQ ID NO: 14 herein having the following substitutions G128D+D143N (activity ratio in AGU:AGU:FAU-F is about 20:5:1).
Glucoamylase BL (GBL): Blend comprising Trametes cingulata glucoamylase disclosed as SEQ ID NO: 2 in WO 06/69289 or SEQ ID NO: 13 herein, and Rhizomucor pusillus alpha-amylase with Aspergillus niger glucoamylase linker and starch binding domain (SBD) disclosed in SEQ ID NO: 14 herein having the following substitutions G128D+D143N (activity ratio in AGU:AGU:FAU-F is about 20:5:1).
Protease X: Metallo protease derived from Thermoascus aurantiacus CGMCC No. 0670 disclosed as amino acids 1-177 in SEQ ID NO: 6 herein and amino acids 1-177 in SEQ ID NO: 2 in WO 2003/048353 Yeast: ETHANOL RED™ available from Red Star/Lesaffre, USA.

### Methods

### Determination of Td by Differential Scanning Calorimetry for Xylanases.

The thermostability of an enzyme is determined by Differential Scanning Calorimetry (DSC) using a VP-Capillary Differential Scanning Calorimeter (MicroCal Inc., Piscataway, N.J., USA). The thermal denaturation temperature, Td (° C.), is taken as the top of denaturation peak (major endothermic peak) in thermograms (Cp vs. T) obtained after heating enzyme solutions (approx. 0.5 mg/ml) in buffer (50 mM acetate, pH 5.0) at a constant programmed heating rate of 200 K/hr.

Sample- and reference-solutions (approx. 0.2 ml) are loaded into the calorimeter (reference: buffer without enzyme) from storage conditions at 10° C. and thermally pre-equilibrated for 20 minutes at 20° C. prior to DSC scan from 20° C. to 120° C. Denaturation temperatures are determined at an accuracy of approximately +/−1° C.

Identity: The relatedness between two amino acid sequences or between two nucleotide sequences is described by the parameter “identity”.

For purposes of the present invention the degree of identity between two amino acid sequences, as well as the degree of identity between two nucleotide sequences, may be determined by the program “align” which is a Needleman-Wunsch alignment (i.e. a global alignment). The program is used for alignment of polypeptide, as well as nucleotide sequences. The default scoring matrix BLOSUM50 is used for polypeptide alignments, and the default identity matrix is used for nucleotide alignments. The penalty for the first residue of a gap is −12 for polypeptides and −16 for nucleotides. The penalties for further residues of a gap are −2 for polypeptides, and −4 for nucleotides.

“Align” is part of the FASTA package version v20u6 (see W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA,” Methods in Enzymology 183:63-98). FASTA protein alignments use the Smith-Waterman algorithm with no limitation on gap size (see “Smith-Waterman algorithm”, T. F. Smith and M. S. Waterman (1981) J. Mol. Biol. 147:195-197).

### Zein-BCA Assay:

Zein-BCA assay can be performed to detect soluble protein quantification released from zein by variant proteases at various temperatures.

### Protocol:

1□ Mix 10 microliters of 10 micrograms/ml enzyme solutions and 100u1 of 0.025% zein solution in a micro titer plate (MTP).
2□ Incubate at various temperatures for 60 min.
3□ Add 10 microliters of 100% trichloroacetic acid (TCA) solution.
4□ Centrifuge MTP at 3500 rpm for 5 min.
5□ Take out 15 microliters to a new MTP containing 100 microliters of BCA assay solution (Pierce Cat #: 23225, BCA Protein Assay Kit).
6□ Incubate for 30 min. at 60° C.
7□ Measure A562.

The results of the assay can be used to determine proteases with improved thermo-stability as compared to a reference protease.

### Determination of Pullulanase Activity (NPUN)

Endo-pullulanase activity in NPUN is measured relative to a Novozymes pullulanase standard. One pullulanase unit (NPUN) is defined as the amount of enzyme that releases 1 micro mol glucose per minute under the standard conditions (0.7% red pullulan (Megazyme), pH 5, 40° C., 20 minutes). The activity is measured in NPUN/ml using red pullulan.

1 mL diluted sample or standard is incubated at 40° C. for 2 minutes. 0.5 mL 2% red pullulan, 0.5 M KCl, 50 mM citric acid, pH 5 are added and mixed. The tubes are incubated at 40° C. for 20 minutes and stopped by adding 2.5 ml 80% ethanol. The tubes are left standing at room temperature for 10-60 minutes followed by centrifugation 10 minutes at 4000 rpm. OD of the supernatants is then measured at 510 nm and the activity calculated using a standard curve.

### AZCL-Casein Assay

A solution of 0.2% of the blue substrate AZCL-casein is suspended in Borax/NaH₂PO₄ buffer pH9 while stirring. The solution is distributed while stirring to microtiter plate (100 microL to each well), 30 microL enzyme sample is added and the plates are incubated in an Eppendorf Thermomixer for 30 minutes at 45° C. and 600 rpm. Denatured enzyme sample (100° C. boiling for 20 min) is used as a blank. After incubation the reaction is stopped by transferring the microtiter plate onto ice and the coloured solution is separated from the solid by centrifugation at 3000 rpm for 5 minutes at 4° C. 60 microL of supernatant is transferred to a microtiter plate and the absorbance at 595 nm is measured using a BioRad Microplate Reader.

### Protease Assays

### AZCL-Casein Assay

A solution of 0.2% of the blue substrate AZCL-casein is suspended in Borax/NaH₂PO₄ buffer pH9 while stirring. The solution is distributed while stirring to microtiter plate (100 microL to each well), 30 microL enzyme sample is added and the plates are incubated in an Eppendorf Thermomixer for 30 minutes at 45° C. and 600 rpm. Denatured enzyme sample (100° C. boiling for 20 min) is used as a blank. After incubation the reaction is stopped by transferring the microtiter plate onto ice and the coloured solution is separated from the solid by centrifugation at 3000 rpm for 5 minutes at 4° C. 60 microL of supernatant is transferred to a microtiter plate and the absorbance at 595 nm is measured using a BioRad Microplate Reader.

pNA-Assay

50 microL protease-containing sample is added to a microtiter plate and the assay is started by adding 100 microL 1 mM pNA substrate (5 mg dissolved in 100 microL DMSO and further diluted to 10 mL with Borax/NaH₂PO₄ buffer pH 9.0). The increase in OD₄₀₅ at room temperature is monitored as a measure of the protease activity.

### Glucoamylase Activity (AGU)

Glucoamylase activity may be measured in Glucoamylase Units (AGU).

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| AMG incubation: | |
|---|---|
| Substrate: | maltose 23.2 mM |
| Buffer: | acetate 0.1M |
| pH: | 4.30 ± 0.05 |
| Incubation temperature: | 37° C. ± 1 |
| Reaction time: | 5 minutes |
| Enzyme working range: | 0.5-4.0 AGU/mL |

| Color reaction: | |
|---|---|
| GlucDH: | 430 U/L |
| Mutarotase: | 9 U/L |
| NAD: | 0.21 mM |
| Buffer: | phosphate 0.12M; 0.15M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

### Acid Alpha-Amylase Activity (AFAU)

Acid alpha-amylase activity may be measured in AFAU (Acid Fungal Alpha-amylase Units), which are determined relative to an enzyme standard. 1 AFAU is defined as the amount of enzyme which degrades 5.260 mg starch dry matter per hour under the below mentioned standard conditions.

Acid alpha-amylase, an endo-alpha-amylase (1,4-alpha-D-glucan-glucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

$${STARCH + IODINE}_{λ = 590 nm} {{\to }^{ALPHA ‐ AMYLASE}}_{40 ° , pH 2 , 5} DEXTRINS + OLIGOSACCHARIDES \\ blue / violet t = 23 sec . decoloration$$

Standard Conditions/Reaction Conditions:

Substrate: Soluble starch, approx. 0.17 g/L

Buffer: Citrate, approx. 0.03 M

Iodine (I2): 0.03 g/L

CaCl2: 1.85 mM

pH: 2.50±0.05

Incubation temperature: 40° C.

Reaction time: 23 seconds

Wavelength: 590 nm

Enzyme concentration: 0.025 AFAU/mL

Enzyme working range: 0.01-0.04 AFAU/mL

A folder EB-SM-0259,02/01 describing this analytical method in more detail is available upon request to Novozymes A/S, Denmark, which folder is hereby included by reference.

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum soluble.

A folder EB-SM-0009.02/01 describing this analytical method in more detail is available upon request to Novozymes A/S, Denmark, which folder is hereby included by reference.

### Determination of FAU(F)

FAU(F) Fungal Alpha-Amylase Units (Fungamyl) is measured relative to an enzyme standard of a declared strength.

Reaction conditions

| Temperature | 37° | C. |
|---|---|---|
| pH | 7.15 | |
| Wavelength | 405 | nm |
| Reaction time | 5 | min |
| Measuring time | 2 | min |

A folder (EB-SM-0216.02) describing this standard method in more detail is available on request from Novozymes A/S, Denmark, which folder is hereby included by reference.

### Determination of Pullulanase Activity (NPUN)

Endo-pullulanase activity in NPUN is measured relative to a Novozymes pullulanase standard. One pullulanase unit (NPUN) is defined as the amount of enzyme that releases 1 micro mol glucose per minute under the standard conditions (0.7% red pullulan (Megazyme), pH 5, 40° C., 20 minutes). The activity is measured in NPUN/ml using red pullulan.

1 mL diluted sample or standard is incubated at 40° C. for 2 minutes. 0.5 mL 2% red pullulan, 0.5 M KCl, 50 mM citric acid, pH 5 are added and mixed. The tubes are incubated at 40° C. for 20 minutes and stopped by adding 2.5 ml 80% ethanol. The tubes are left standing at room temperature for 10-60 minutes followed by centrifugation 10 minutes at 4000 rpm. OD of the supernatants is then measured at 510 nm and the activity calculated using a standard curve.

The present invention is described in further detail in the following examples which are offered to illustrate the present invention, but not in any way intended to limit the scope of the invention as claimed. All references cited herein are specifically incorporated by reference for that which is described therein.

### EXAMPLES

### Example 1—Stability of Alpha-Amylase Variants

The stability of a reference alpha-amylase (Bacillus stearothermophilus alpha-amylase with the mutations I181*+G182*+N193F truncated to 491 amino acids (SEQ ID NO: 1 numbering)) and alpha-amylase variants thereof was determined by incubating the reference alpha-amylase and variants at pH 4.5 and 5.5 and temperatures of 75° C. and 85° C. with 0.12 mM CaCl₂ followed by residual activity determination using the EnzChek® substrate (EnzChek® Ultra Amylase assay kit, E33651, Molecular Probes).

Purified enzyme samples were diluted to working concentrations of 0.5 and 1 or 5 and 10 ppm (micrograms/ml) in enzyme dilution buffer (10 mM acetate, 0.01% Triton X100, 0.12 mM CaCl₂, pH 5.0). Twenty microliters enzyme sample was transferred to 48-well PCR MTP and 180 microliters stability buffer (150 mM acetate, 150 mM MES, 0.01% Triton X100, 0.12 mM CaCl₂, pH 4.5 or 5.5) was added to each well and mixed. The assay was performed using two concentrations of enzyme in duplicates. Before incubation at 75° C. or 85° C., 20 microliters was withdrawn and stored on ice as control samples. Incubation was performed in a PCR machine at 75° C. and 85° C. After incubation samples were diluted to 15 ng/mL in residual activity buffer (100 mM Acetate, 0.01% Triton X100, 0.12 mM CaCl₂, pH 5.5) and 25 microliters diluted enzyme was transferred to black 384-MTP. Residual activity was determined using the EnzChek substrate by adding 25 microliters substrate solution (100 micrograms/ml) to each well. Fluorescence was determined every minute for 15 minutes using excitation filter at 485-P nm and emission filter at 555 nm (fluorescence reader is Polarstar, BMG). The residual activity was normalized to control samples for each setup.

Assuming logarithmic decay half life time (T % (min)) was calculated using the equation:

T½ (min)=T(min)*LN(0.5)/LN(% RA/100), where T is assay incubation time in minutes, and % RA is % residual activity determined in assay.

Using this assay setup the half life time was determined for the reference alpha-amylase and variant thereof as shown in Table 1.

TABLE 1

| | | T½ (min) | |
|---|---|---|---|
| | T½ (min) | (pH 4.5, 85° C., | T½ (min) |
| | (pH 4.5, 75° C., | 0.12 mM | (pH 5.5, 85° C., |
| Mutations | 0.12 mM CaCl₂) | CaCl₂) | 0.12 mM CaCl₂) |
| Reference Alpha-Amylase A | 21 | 4 | 111 |
| Reference Alpha-Amylase A with | 32 | 6 | 301 |
| the substitution V59A | | | |
| Reference Alpha-Amylase A with | 28 | 5 | 230 |
| the substitution V59E | | | |
| Reference Alpha-Amylase A with | 28 | 5 | 210 |
| the substitution V59I | | | |
| Reference Alpha-Amylase A with | 30 | 6 | 250 |
| the substitution V59Q | | | |
| Reference Alpha-Amylase A with | 149 | 22 | ND |
| the substitutions V59A + Q89R + | | | |
| G112D + E129V + K177L + R179E + | | | |
| K220P + N224L + Q254S | | | |
| Reference Alpha-Amylase A with | >180 | 28 | ND |
| the substitutions | | | |
| V59A + Q89R + E129V + | | | |
| K177L + R179E + H208Y + K220P + | | | |
| N224L + Q254S | | | |
| Reference Alpha-Amylase A with | 112 | 16 | ND |
| the substitutions | | | |
| V59A + Q89R + E129V + | | | |
| K177L + R179E + K220P + N224L + | | | |
| Q254S + D269E + D281N | | | |
| Reference Alpha-Amylase A with | 168 | 21 | ND |
| the substitutions | | | |
| V59A + Q89R + E129V + | | | |
| K177L + R179E + K220P + N224L + | | | |
| Q254S + I270L | | | |
| Reference Alpha-Amylase A with | >180 | 24 | ND |
| the substitutions | | | |
| V59A + Q89R + E129V + | | | |
| K177L + R179E + K220P + N224L + | | | |
| Q254S + H274K | | | |
| Reference Alpha-Amylase A with | 91 | 15 | ND |
| the substitutions | | | |
| V59A + Q89R + E129V + | | | |
| K177L + R179E + K220P + N224L + | | | |
| Q254S + Y276F | | | |
| Reference Alpha-Amylase A with | 141 | 41 | ND |
| the substitutions V59A + E129V + | | | |
| R157Y + K177L + R179E + K220P + | | | |
| N224L + S242Q + Q254S | | | |
| Reference Alpha-Amylase A with | >180 | 62 | ND |
| the substitutions V59A + E129V + | | | |
| K177L + R179E + H208Y + K220P + | | | |
| N224L + S242Q + Q254S | | | |
| Reference Alpha-Amylase A with | >180 | 49 | >480 |
| the substitutions V59A + E129V + | | | |
| K177L + R179E + K220P + N224L + | | | |
| S242Q + Q254S | | | |
| Reference Alpha-Amylase A with | >180 | 53 | ND |
| the substitutions V59A + E129V + | | | |
| K177L + R179E + K220P + N224L+ | | | |
| S242Q + Q254S + H274K | | | |
| Reference Alpha-Amylase A with | >180 | 57 | ND |
| the substitutions V59A + E129V + | | | |
| K177L + R179E + K220P + N224L + | | | |
| S242Q + Q254S + Y276F | | | |
| Reference Alpha-Amylase A with | >180 | 37 | ND |
| the substitutions V59A + E129V + | | | |
| K177L + R179E + K220P + N224L + | | | |
| S242Q + Q254S + D281N | | | |
| Reference Alpha-Amylase A with | >180 | 51 | ND |
| the substitutions V59A + E129V + | | | |
| K177L + R179E + K220P + N224L + | | | |
| S242Q + Q254S + M284T | | | |
| Reference Alpha-Amylase A with | >180 | 45 | ND |
| the substitutions V59A + E129V + | | | |
| K177L + R179E + K220P + N224L + | | | |
| S242Q + Q254S + G416V | | | |
| Reference Alpha-Amylase A with | 143 | 21 | >480 |
| the substitutions V59A + E129V + | | | |
| K177L + R179E + K220P + N224L + | | | |
| Q254S | | | |
| Reference Alpha-Amylase A with | >180 | 22 | ND |
| the substitutions V59A + E129V + | | | |
| K177L + R179E + K220P + N224L + | | | |
| Q254S + M284T | | | |
| Reference Alpha-Amylase A with | >180 | 38 | ND |
| the substitutions | | | |
| A91L + M96I + E129V + | | | |
| K177L + R179E + K220P + N224L + | | | |
| S242Q + Q254S | | | |
| Reference Alpha-Amylase A with | 57 | 11 | 402 |
| the substitutions E129V + K177L + | | | |
| R179E | | | |
| Reference Alpha-Amylase A with | 174 | 44 | >480 |
| the substitutions E129V + K177L + | | | |
| R179E + K220P + N224L + S242Q + | | | |
| Q254S | | | |
| Reference Alpha-Amylase A with | >180 | 49 | >480 |
| the substitutions E129V + K177L + | | | |
| R179E + K220P + N224L + S242Q + | | | |
| Q254S + Y276F + L427M | | | |
| Reference Alpha-Amylase A with | >180 | 49 | >480 |
| the substitutions E129V + K177L + | | | |
| R179E + K220P + N224L + S242Q + | | | |
| Q254S + M284T | | | |
| Reference Alpha-Amylase A with | 177 | 36 | >480 |
| the substitutions E129V + K177L + | | | |
| R179E + K220P + N224L + S242Q + | | | |
| Q254S + N376* + I377* | | | |
| Reference Alpha-Amylase A with | 94 | 13 | >480 |
| the substitutions E129V + K177L + | | | |
| R179E + K220P + N224L + Q254S | | | |
| Reference Alpha-Amylase A with | 129 | 24 | >480 |
| the substitutions E129V + K177L + | | | |
| R179E + K220P + N224L + Q254S + | | | |
| M284T | | | |
| Reference Alpha-Amylase A with | 148 | 30 | >480 |
| the substitutions E129V + K177L + | | | |
| R179E + S242Q | | | |
| Reference Alpha-Amylase A with | 78 | 9 | >480 |
| the substitutions E129V + K177L + | | | |
| R179V | | | |
| Reference Alpha-Amylase A with | 178 | 31 | >480 |
| the substitutions E129V + K177L + | | | |
| R179V + K220P + N224L + S242Q + | | | |
| Q254S | | | |
| Reference Alpha-Amylase A with | 66 | 17 | >480 |
| the substitutions K220P + N224L + | | | |
| S242Q + Q254S | | | |
| Reference Alpha-Amylase A with | 30 | 6 | 159 |
| the substitutions K220P + N224L + | | | |
| Q254S | | | |
| Reference Alpha-Amylase A with | 35 | 7 | 278 |
| the substitution M284T | | | |
| Reference Alpha-Amylase A with | 59 | 13 | ND |
| the substitutions M284V | | | |

ND not determined

The results demonstrate that the alpha-amylase variants have a significantly greater half-life and stability than the reference alpha-amylase.

### Example 2—Preparation of Protease Variants and Test of Thermostability Strains and Plasmids

E. coli DH12S (available from Gibco BRL) was used for yeast plasmid rescue. pJTP000 is a S. cerevisiae and E. coli shuttle vector under the control of TPI promoter, constructed from pJC039 described in WO 01/92502, in which the Thermoascus aurantiacus M35 protease gene (WO 03048353) has been inserted.

Saccharomyces cerevisiae YNG318 competent cells: MATa Dpep4[cir+] ura3-52, leu2-D2, his 4-539 was used for protease variants expression. It is described in J. Biol. Chem. 272 (15), pp 9720-9727, 1997.

### Media and Substrates

10× Basal solution: Yeast nitrogen base w/o amino acids (DIFCO) 66.8 g/l, succinate 100 g/l, NaOH 60 g/l.
SC-glucose: 20% glucose (i.e., a final concentration of 2%=2 g/100 ml)) 100 ml/l, 5% threonine 4 ml/l, 1% tryptophan 10 ml/l, 20% casamino acids 25 ml/l, 10× basal solution 100 ml/l. The solution is sterilized using a filter of a pore size of 0.20 micrometer. Agar (2%) and H₂O (approx. 761 ml) is autoclaved together, and the separately sterilized SC-glucose solution is added to the agar solution.
YPD: Bacto peptone 20 g/l, yeast extract 10 g/l, 20% glucose 100 ml/l.

### YPD+Zn: YPD+0.25 mM ZnSO₄.

PEG/LiAc solution: 40% PEG4000 50 ml, 5 M Lithium Acetate 1 ml.

### 96 Well Zein Micro Titre Plate:

Each well contains 200 microL of 0.05-0.1% of zein (Sigma), 0.25 mM ZnSO₄ and 1% of agar in 20 mM sodium acetate buffer, pH 4.5.

### DNA Manipulations

Unless otherwise stated, DNA manipulations and transformations were performed using standard methods of molecular biology as described in Sambrook et al. (1989) Molecular cloning: A laboratory manual, Cold Spring Harbor lab. Cold Spring Harbor, N.Y.; Ausubel, F. M. et al. (eds.) “Current protocols in Molecular Biology”, John Wiley and Sons, 1995; Harwood, C. R. and Cutting, S. M. (Eds.).

### Yeast Transformation

Yeast transformation was performed using the lithium acetate method. 0.5 microL of vector (digested by restriction endonucleases) and 1 microL of PCR fragments is mixed. The DNA mixture, 100 microL of YNG318 competent cells, and 10 microL of YEAST MAKER carrier DNA (Clontech) is added to a 12 ml polypropylene tube (Falcon 2059). Add 0.6 ml PEG/LiAc solution and mix gently. Incubate for 30 min at 30° C., and 200 rpm followed by 30 min at 42° C. (heat shock). Transfer to an eppendorf tube and centrifuge for 5 sec. Remove the supernatant and resolve in 3 ml of YPD. Incubate the cell suspension for 45 min at 200 rpm at 30° C. Pour the suspension to SC-glucose plates and incubate 30° C. for 3 days to grow colonies. Yeast total DNA are extracted by Zymoprep Yeast Plasmid Miniprep Kit (ZYMO research).

### DNA Sequencing

E. coli transformation for DNA sequencing was carried out by electroporation (BIO-RAD Gene Pulser). DNA Plasmids were prepared by alkaline method (Molecular Cloning, Cold Spring Harbor) or with the Qiagen® Plasmid Kit. DNA fragments were recovered from agarose gel by the Qiagen gel extraction Kit. PCR was performed using a PTC-200 DNA Engine. The ABI PRISM™ 310 Genetic Analyzer was used for determination of all DNA sequences.

### Construction of Protease Expression Vector

The Thermoascus M35 protease gene was amplified with the primer pair Prot F (SEQ ID NO: 4) and Prot R (SEQ ID NO: 5). The resulting PCR fragments were introduced into S. cerevisiae YNG318 together with the pJC039 vector (described in WO 2001/92502) digested with restriction enzymes to remove the Humicola insolens cutinase gene.

The Plasmid in yeast clones on SC-glucose plates was recovered to confirm the internal sequence and termed as pJTP001.

### Construction of Yeast Library and Site-Directed Variants

Library in yeast and site-directed variants were constructed by SOE PCR method (Splicing by Overlap Extension, see “PCR: A practical approach”, p. 207-209, Oxford University press, eds. McPherson, Quirke, Taylor), followed by yeast in vivo recombination.

### General Primers for Amplification and Sequencing

The primers AM34 (SEQ ID NO:5) and AM35 (SEQ ID NO:6) were used to make DNA fragments containing any mutated fragments by the SOE method together with degenerated primers (AM34+Reverse primer and AM35+forward primer) or just to amplify a whole protease gene (AM34+AM35).

| PCR reaction system: | Conditions: | |
|---|---|---|
| 48.5 microL H₂O | 1 | 94° C. 2 min |
| 2 beads puRe Taq Ready-To-Go PCR | 2 | 94° C. 30 sec |
| (Amersham Biosciences) | 3 | 55° C. 30 sec |
| 0.5 micro L X 2 100 pmole/microL of primers | 4 | 72° C. 90 sec |
| 0.5 microL template DNA | 2-4 | 25 cycles |
| | 5 | 72° C. 10 min |

DNA fragments were recovered from agarose gel by the Qiagen gel extraction Kit. The resulting purified fragments were mixed with the vector digest. The mixed solution was introduced into Saccharomyces cerevisiae to construct libraries or site-directed variants by in vivo recombination.

### Relative Activity Assay

Yeast clones on SC-glucose were inoculated to a well of a 96-well micro titre plate containing YPD+Zn medium and cultivated at 28° C. for 3 days. The culture supernatants were applied to a 96-well zein micro titer plate and incubated at at least 2 temperatures (ex. 60° C. and 65° C., 70° C. and 75° C., 70° C. and 80° C.) for more than 4 hours or overnight. The turbidity of zein in the plate was measured as A630 and the relative activity (higher/lower temperatures) was determined as an indicator of thermoactivity improvement. The clones with higher relative activity than the parental variant were selected and the sequence was determined.

### Remaining Activity Assay

Yeast clones on SC-glucose were inoculated to a well of a 96-well micro titre plate and cultivated at 28° C. for 3 days. Protease activity was measured at 65° C. using azo-casein (Megazyme) after incubating the culture supernatant in 20 mM sodium acetate buffer, pH 4.5, for 10 min at a certain temperature (80° C. or 84° C. with 4° C. as a reference) to determine the remaining activity. The clones with higher remaining activity than the parental variant were selected and the sequence was determined.

### Azo-Casein Assay

20 microL of samples were mixed with 150 microL of substrate solution (4 ml of 12.5% azo-casein in ethanol in 96 ml of 20 mM sodium acetate, pH 4.5, containing 0.01% triton-100 and 0.25 mM ZnSO₄) and incubated for 4 hours or longer.

After adding 20 microL/well of 100% trichloroacetic acid (TCA) solution, the plate was centrifuge and 100 microL of supernatants were pipette out to measure A440.

Expression of Protease Variants in Aspergillus oryzae

The constructs comprising the protease variant genes were used to construct expression vectors for Aspergillus. The Aspergillus expression vectors consist of an expression cassette based on the Aspergillus niger neutral amylase II promoter fused to the Aspergillus nidulans triose phosphate isomerase non translated leader sequence (Pna2/tpi) and the Aspergillus niger amyloglucosidase terminator (Tamg). Also present on the plasmid was the Aspergillus selective marker amdS from Aspergillus nidulans enabling growth on acetamide as sole nitrogen source. The expression plasmids for protease variants were transformed into Aspergillus as described in Lassen et al. (2001), Appl. Environ. Microbiol. 67, 4701-4707. For each of the constructs 10-20 strains were isolated, purified and cultivated in shake flasks.

### Purification of Expressed Variants

1. Adjust pH of the 0.22 μm filtered fermentation sample to 4.0.
2. Put the sample on an ice bath with magnetic stirring. Add (NH4)2SO4 in small aliquots (corresponding to approx. 2.0-2.2 M (NH4)₂SO₄ not taking the volume increase into account when adding the compound).
3. After the final addition of (NH4)2SO4, incubate the sample on the ice bath with gentle magnetic stirring for min. 45 min.
4. Centrifugation: Hitachi himac CR20G High-Speed Refrigerated Centrifuge equipped with R20A2 rotor head, 5° C., 20,000 rpm, 30 min.
5. Dissolve the formed precipitate in 200 ml 50 mM Na-acetate pH 4.0.
6. Filter the sample by vacuum suction using a 0.22 μm PES PLUS membrane (IWAKI).
7. Desalt/buffer-exchange the sample to 50 mM Na-acetate pH 4.0 using ultrafiltration (Vivacell 250 from Vivascience equipped with 5 kDa MWCO PES membrane) overnight in a cold room. Dilute the retentate sample to 200 ml using 50 mM Na-acetate pH 4.0. The conductivity of sample is preferably less than 5 mS/cm.
8. Load the sample onto a cation-exchange column equilibrated with 50 mM Na-acetate pH 4.0. Wash unbound sample out of the column using 3 column volumes of binding buffer (50 mM Na-acetate pH 4.0), and elute the sample using a linear gradient, 0-100% elution buffer (50 mM Na-acetate+1 M NaCl pH 4.0) in 10 column volumes.
9. The collected fractions are assayed by an endo-protease assay (cf. below) followed by standard SDS-PAGE (reducing conditions) on selected fractions. Fractions are pooled based on the endo-protease assay and SDS-PAGE.

### Endo-Protease Assay

1. Protazyme OL tablet/5 ml 250 mM Na-acetate pH 5.0 is dissolved by magnetic stirring (substrate: endo-protease Protazyme AK tablet from Megazyme—cat. #PRAK 11/08).
2. With stirring, 250 microL of substrate solution is transferred to a 1.5 ml Eppendorf tube.
3. 25 microL of sample is added to each tube (blank is sample buffer).
4. The tubes are incubated on a Thermomixer with shaking (1000 rpm) at 50° C. for 15 minutes.
5. 250 microL of 1 M NaOH is added to each tube, followed by vortexing.
6. Centrifugation for 3 min. at 16,100×G and 25° C.
7. 200 microL of the supernatant is transferred to a MTP, and the absorbance at 590 nm is recorded.

### Results

TABLE 2
Relative activity of protease variants. Numbering of
substitution(s) starts from N-terminal of the mature peptide
in amino acids 1 to 177 of SEQ ID NO: 2.

| | | Relative activity |
|---|---|---|
| Variant | Substitution(s) | 65° C./60° C. |
| WT | none | 31% |
| JTP004 | S87P | 45% |
| JTP005 | A112P | 43% |
| JTP008 | R2P | 71% |
| JTP009 | D79K | 69% |
| JTP010 | D79L | 75% |
| JTP011 | D79M | 73% |
| JTP012 | D79L/S87P | 86% |
| JTP013 | D79L/S87P/A112P | 90% |
| JTP014 | D79L/S87P/A112P | 88% |
| JTP016 | A73C | 52% |
| JTP019 | A126V | 69% |
| JTP021 | M152R | 59% |

TABLE 3
Relative activity of protease variants. Numbering of substitution(s) starts from
N-terminal of the mature peptide in amino acids 1 to 177 of SEQ ID NO: 2.

| | | Relative activity | | |
|---|---|---|---|---|
| Variant | Substitution(s) and/or deletion (S) | 70° C./65° C. | 75° C./65° C. | 75° C./70° C. |
| WT | none | 59% | 17% | |
| JTP036 | D79L/S87P/D142L | 73% | 73% | |
| JTP040 | T54R/D79L/S87P | | 71% | |
| JTP042 | Q53K/D79L/S87P/I173V | | 108% | |
| JTP043 | Q53R/D79L/S87P | | 80% | |
| JTP045 | S41R/D79L/S87P | | 82% | |
| JTP046 | D79L/S87P/Q158W | | 96% | |
| JTP047 | D79L/S87P/S157K | | 85% | |
| JTP048 | D79L/S87P/D104R | | 88% | |
| JTP050 | D79L/S87P/A112P/D142L | | 88% | |
| JTP051 | S41R/D79L/S87P/A112P/D142L | | | 102% |
| JTP052 | D79L/S87P/A112P/D142L/S157K | | | 111% |
| JTP053 | S41R/D79L/S87P/A112P/D142L/S157K | | | 113% |
| JTP054 | ΔS5/D79L/S87P | | | 92% |
| JTP055 | ΔG8/D79L/S87P | | | 95% |
| JTP059 | C6R/D79L/S87P | | | 92% |
| JTP061 | T46R/D79L/S87P | | | 111% |
| JTP063 | S49R/D79L/S87P | | | 94% |
| JTP064 | D79L/S87P/N88R | | | 92% |
| JTP068 | D79L/S87P/T114P | | | 99% |
| JTP069 | D79L/S87P/S115R | | | 103% |
| JTP071 | D79L/S87P/T116V | | | 105% |
| JTP072 | N26R/D79L/S87P | | 92% | |
| JTP077 | A27K/D79L/S87P/A112P/D142L | | 106% | |
| JTP078 | A27V/D79L/S87P/A112P/D142L | | 100% | |
| JTP079 | A27G/D79L/S87P/A112P/D142L | | 104% | |

TABLE 4
Relative activity of protease variants. Numbering of substitution(s) starts from
N-terminal of the mature peptide in amino acids 1 to 177 of SEQ ID NO: 2.

| | | Relative | Remaining | |
|---|---|---|---|---|
| | | activity | activity | |
| Variant | Substitution(s) and/or deletion(s) | 75° C./65° C. | 80° C. | 84° C. |
| JTP082 | ΔS5/D79L/S87P/A112P/D142L | 129% | | 53% |
| JTP083 | T46R/D79L/S87P/A112P/D142L | 126% | | |
| JTP088 | Y43F/D79L/S87P/A112P/D142L | 119% | | |
| JTP090 | D79L/S87P/A112P/T124L/D142L | 141% | | |
| JTP091 | D79L/S87P/A112P/T124V/D142L | 154% | 43% | |
| JTP092 | ΔS5/N26R/D79L/S87P/A112P/D142L | | | 60% |
| JTP095 | N26R/T46R/D79L/S87P/A112P/D142L | | | 62% |
| JTP096 | T46R/D79L/S87P/T116V/D142L | | | 67% |
| JTP099 | D79L/P81R/S87 P/A112 P/D142 L | | | 80% |
| JTP101 | A27K/D79L/S87P/A112P/T124V/D142L | | 81% | |
| JTP116 | D79L/Y82F/S87P/A112P/T124V/D142L | | 59% | |
| JTP117 | D79L/Y82F/S87P/A112P/T124V/D142L | | 94% | |
| JTP127 | D79L/S87P/A112P/T124V/A126V/D142L | | 53% | |

TABLE5
Relative activity of protease variants. Numbering of substitution(s) starts from
N-terminal of the mature peptide in amino acids 1 to 177 of SEQ ID NO: 2.

| | | Relative activity | | |
|---|---|---|---|---|
| Variant | Substitutions | 75° C./70° C. | 80° C./70° C. | 85° C./70° C. |
| JTP050 | D79L S87P A112P D142L | 55% | 23% | 9% |
| JTP134 | D79L Y82F S87P A112P D142L | | 40% | |
| JTP135 | S38T D79L S87P A112P A126V D142L | | 62% | |
| JTP136 | D79L Y82F S87P A112P A126V D142L | | 59% | |
| JTP137 | A27K D79L S87P A112P A126V D142L | | 54% | |
| JTP140 | D79L S87P N98C A112P G135C D142L | 81% | | |
| JTP141 | D79L S87P A112P D142L T141C M161C | 68% | | |
| JTP143 | S36P D79L S87P A112P D142L | 69% | | |
| JTP144 | A37P D79L S87P A112P D142L | 57% | | |
| JTP145 | S49P D79L S87P A112P D142L | 82% | 59% | |
| JTP146 | S50P D79L S87P A112P D142L | 83% | 63% | |
| JTP148 | D79L S87P D104P A112P D142L | 76% | 64% | |
| JTP161 | D79L Y82F S87G A112P D142L | | 30% | 12% |
| JTP180 | S70V D79L Y82F S87G Y97W A112P | | 52% | |
| | D142L | | | |
| JTP181 | D79L Y82F S87G Y97W D104P A112P | | 45% | |
| | D142L | | | |
| JTP187 | S70V D79L Y82F S87G A112P D142L | | 45% | |
| JTP188 | D79L Y82F S87G D104P A112P D142L | | 43% | |
| JTP189 | D79L Y82F S87G A112P A126V D142L | | 46% | |
| JTP193 | Y82F S87G S70V D79L D104P A112P | | | 15% |
| | D142L | | | |
| JTP194 | Y82F S87G D79L D104P A112P A126V | | | 22% |
| | D142L | | | |
| JTP196 | A27K D79L Y82F S87G D104P A112P | | | 18% |
| | A126V D142L | | | |

TABLE 5
Relative activity of protease variants. Numbering
of substitution(s) starts from N-terminal of the mature
peptide in amino acids 1 to 177 of SEQ ID NO: 2.

| | | Relative activity | |
|---|---|---|---|
| Variant | Substitutions | 75°C/70°C | 80°C/70°C |
| JTP196 | A27K D79L Y82F | 102% | 55% |
| | S87G D104P A112P | | |
| | A126V D142L | | |
| JTP210 | A27K Y82F S87G | 107% | 36% |
| | D104P A112P A126V | | |
| | D142L | | |
| JTP211 | A27K D79L Y82F | 94% | 44% |
| | D104P A112P A126V | | |
| | D142L | | |
| JTP213 | A27K Y82F D104P | 103% | 37% |
| | A112P A126V D142L | | |

### Example 3

### Temperature Profile of Selected Variants Using Purified Enzymes

Selected variants showing good thermo-stability were purified and the purified enzymes were used in a zein-BCA assay as described below. The remaining protease activity was determined at 60° C. after incubation of the enzyme at elevated temperatures as indicated for 60 min.

### Zein-BCA Assay:

Zein-BCA assay was performed to detect soluble protein quantification released from zein by variant proteases at various temperatures.

### Protocol:

8□ Mix 10u1 of 10 ug/ml enzyme solutions and 100u1 of 0.025% zein solution in a micro titer plate (MTP).
9□ Incubate at various temperatures for 60 min.
10□ Add 10u1 of 100% trichloroacetic acid (TCA) solution.
11□ Centrifuge MTP at 3500 rpm for 5 min.
12□ Take out 15u1 to a new MTP containing 100u1 of BCA assay solution (Pierce Cat #: 23225, BCA Protein Assay Kit).
13□ Incubate for 30 min. at 60° C.
14□ Measure A562.
   The results are shown in Table 6. All of the tested variants showed an improved thermo-stability as compared to the wt protease.

TABLE 6
Zein-BCA assay

| | Sample incubated 60 min at indicated temperatures (° C.) | | | | | | |
|---|---|---|---|---|---|---|---|
| | (μg/ml Bovine serum albumin equivalent peptide released) | | | | | | |
| WT/Variant | 60° C. | 70° C. | 75° C. | 80° C. | 85° C. | 90° C. | 95° C. |
| WT | 94 | 103 | 107 | 93 | 58 | 38 | |
| JTP050 | 86 | 101 | 107 | 107 | 104 | 63 | 36 |
| JTP077 | 82 | 94 | 104 | 105 | 99 | 56 | 31 |
| JTP188 | 71 | 83 | 86 | 93 | 100 | 75 | 53 |
| JTP196 | 87 | 99 | 103 | 106 | 117 | 90 | 38 |

### Example 4

### Thermostability of Protease Pfu

The thermostability of the Pyrococcus furiosus protease (Pfu S) purchased from Takara Bio Inc, (Japan) was tested using the same methods as in Example 2. It was found that the thermostability (Relative Activity) was 110% at (80° C./70° C.) and 103% (90° C./70° C.) at pH 4.5.

### Example 5: Determine Metal Ions Concentration in Liquefied Corn Mashes

Metal ions analysis was carried out by mean of Ion-Coupled Plasma Optical Emission Spectrometry (ICP-OES). Various liquefied corn mashes were collected from industrial ethanol plants across Midwest USA. The liquefacts were subjected to digestion with Nitric acid (3%, v/v) and the concentration of the respective elements such as Aluminum (Al), Boron (B), Calcium (Ca), Copper (Cu), Iron/Ferrous (Fe), Potassium (K), Magnesium (Mg), Manganese (Mn) Sodium (Na), Nickel (Ni) and Zinc (Zn) were quantified using ICP-OES. Concentrations of the individual elements were calculated using commercially available standard mix from SPEX CertiPrep (Fisher P/N CL-CAL-2).

Weigh 1 g of liquefact sample into a 125 ml graduated Nalgene bottle and fill to 100 ml mark with 3% Nitric acid and mix well. Loosen lids and allow to sit in the vent hood at room temperature for overnight. After overnight digestion, filter 10 ml of the treated mashes through a 0.45 μm syringe filter into a 15 ml centrifuge tube. The filtered samples were put on ICP-OES autosampler from Perkin Elmer model Avio 500. Blank reading was 3% Nitric acid and analysis of each sample was taken average of 3 separate readings.

Results

The metal ions concentration (mM) determined by ICP-OES of various industrial liquefied mashes showed in Table 7. Metal ions such as Ca, Cu, Fe, Mg, Mn, Ni and Zn were selected for characterization study on it effect on the activity of thermostable xylanases from Dictyoglomus thermophilum (SEQ ID NO: 1) and Thermotoga maritima (SEQ ID NO: 2).

TABLE 7

| Liquefied | Concentration (mM) | | | | | | | | | | |
|---|---|---|---|---|---|---|---|---|---|---|---|
| mashes | Al | B | Ca | Cu | Fe | K | Mg | Mn | Na | Ni | Zn |
| Mash 1 | 0.045 | 0.143 | 1.053 | 0.010 | 0.119 | 35.884 | 19.845 | 0.039 | 5.154 | 0.000 | 0.103 |
| Mash 2 | 0.009 | 0.100 | 0.878 | 0.011 | 0.166 | 37.055 | 19.056 | 0.033 | 3.971 | 0.001 | 0.105 |
| Mash 3 | 0.022 | 0.081 | 0.792 | 0.018 | 0.201 | 43.150 | 21.680 | 0.052 | 8.259 | 0.004 | 0.164 |
| Mash 4 | 0.004 | 0.123 | 0.558 | 0.013 | 0.158 | 41.228 | 21.743 | 0.044 | 4.990 | 0.004 | 0.130 |
| Mash 5 | 0.006 | 0.085 | 0.971 | 0.011 | 0.148 | 32.944 | 20.472 | 0.039 | 2.458 | 0.003 | 0.108 |
| Mash 6 | 0.006 | 0.138 | 0.510 | 0.013 | 0.142 | 41.232 | 23.598 | 0.044 | 4.709 | 0.004 | 0.109 |
| Mash 7 | 0.021 | 0.051 | 0.785 | 0.016 | 0.203 | 40.206 | 21.837 | 0.052 | 4.831 | 0.009 | 0.154 |
| Mash 8 | 0.021 | 0.140 | 1.147 | 0.012 | 0.141 | 41.521 | 22.089 | 0.046 | 6.014 | 0.001 | 0.123 |
| Mash 9 | 0.012 | 0.083 | 0.580 | 0.010 | 0.144 | 36.304 | 19.445 | 0.046 | 1.239 | 0.002 | 0.078 |
| Mash 10 | 0.053 | 0.130 | 0.770 | 0.010 | 0.138 | 39.704 | 20.819 | 0.049 | 2.135 | 0.004 | 0.097 |
| Mash 11 | 0.008 | 0.085 | 0.858 | 0.011 | 0.123 | 33.677 | 17.412 | 0.031 | 15.070 | 0.003 | 0.100 |
| Average | 0.019 | 0.105 | 0.809 | 0.012 | 0.153 | 38.446 | 20.727 | 0.043 | 5.348 | 0.003 | 0.116 |
| Minimum | 0.004 | 0.051 | 0.510 | 0.010 | 0.119 | 32.944 | 17.412 | 0.031 | 1.239 | 0.000 | 0.078 |
| Maximum | 0.053 | 0.143 | 1.147 | 0.018 | 0.203 | 43.150 | 23.598 | 0.052 | 15.070 | 0.004 | 0.154 |

### Example 6—Effect of Metal Ions on the Activity of Thermostable Xylanase from Dictyoglomus thermophilum and Thermotoga maritima

The activity of purified thermostable xylanase from Dictyoglomus thermophilum (SEQ ID NO: 1) and Thermotoga maritima (SEQ ID NO: 2) were assayed using 10 g/L wheat arabinoxylan (P-WAXYM, Megazyme) as substrate. The substrate solution was prepared by weighing out 0.5 g of wheat arabinoxylan into a beaker then add approximately 40 ml of MilliQ water. Stir vigorously with heating until 80° C. in microwave oven and repeat stir/heat step until the substrate completely dissolved. Cool down the beaker in water bath while stirring. After the solution is cooled to room temp, transfer to volumetric flask and top up with Milli Q water to 50 ml.

The substrate, buffer and metal ion mixtures were prepared by mixing 250 μl substrate solution, 200 μl 250 mM sodium acetate buffer, pH 5.0 with or without 50 μl metal ion solution of Ca, Cu, Fe, Mg, Mn and Zn at final concentration of 0, 0.0625, 0.125, 0.25 and 0.5 mM into 1.5 ml tube, and vortex. Enzyme activity assay was carried out using PCR thermal cycler. In PCR plate, dispense 80 μl of substrate/buffer/metal ion mix to the wells and reaction was initiated by adding 20 μl of appropriately diluted purified xylanase. Place the cap and incubate at 85° C. for 30 min on Veriti Thermal Cycler (Thermo Fisher Scientific). After 30 min reaction, the plate was immediately cool down in ice for 3 min.

Xylanase generated product of xylo-oligosaccharides was determined using p-hydroxybenzoic acid hydrazide solution (PAHBAH) which detect the amount of reducing sugars released. Add 40 μl of PAHBAH solution to each plate well and incubated at 55° C. for 20 min. After 20 min, transfer 100 μl of the respective PAHBAH reacted supernatant to 96-well plate and measured color developed at 405 nm using spectrophotometer. Enzyme activity was defined as the absorbance intensity at 405 nm. Control was enzyme with substrate/buffer reaction without metal ion presence. Blanks were substrate, buffer and the appropriate concentration of respective metal ion without enzyme addition.

Results

Table 8 showed that Cu, Fe and Zn at concentration as low as 0.0625 mM reduced the activity of Dictyoglomus thermophilum xylanase (SEQ ID NO: 1) by 20 to 40%, whilst in comparison the activity of Thermotoga maritima xylanase (SEQ ID NO: 2) was not affected (Table 9). At higher concentration between 0.125 to 0.25 mM of Fe and Zn which relevant to the amount detected in liquefied corn mashes as shown in Table 7, the activity of Dictyoglomus thermophilum xylanase (SEQ ID NO: 1) was significantly decreased by 50 to 60% (Table 8), compared to Thermotoga maritima xylanase (SEQ ID NO: 2) which was not much affected by Cu or Zn and less impacted by Fe (Table 9).

Unexpectedly, the results described herein suggest that a thermostable xylanase such as Thermotoga maritima xylanase (SEQ ID NO: 2) that is more tolerance to Cu, Fe or Zn inhibition presence in corn, will provide a more robust and consistent performance in liquefaction to deliver yield benefits.

TABLE 8
Effect of metal ions on Dictyoglomus thermophilum xylanase

| Concentration | Relative activity (%) | | | | | |
|---|---|---|---|---|---|---|
| (mM) | Ca | Cu | Fe | Mg | Mn | Zn |
| 0 (Control) | 100% | 100% | 100% | 100% | 100% | 100% |
| 0.0625 | 89% | 56% | 79% | 82% | 91% | 63% |
| 0.125 | 105% | 40% | 57% | 84% | 92% | 57% |
| 0.25 | 114% | 20% | 38% | 91% | 111% | 49% |
| 0.5 | 112% | 14% | 26% | 94% | 114% | 32% |

TABLE 9
Effect of metal ions on Thermotoga maritima xylanase

| Concentration | Relative activity (%) | | | | | |
|---|---|---|---|---|---|---|
| (mM) | Ca | Cu | Fe | Mg | Mn | Zn |
| 0 (Control) | 100% | 100% | 100% | 100% | 100% | 100% |
| 0.0625 | 106% | 91% | 95% | 105% | 108% | 99% |
| 0.125 | 112% | 86% | 73% | 109% | 111% | 98% |
| 0.25 | 111% | 81% | 49% | 108% | 118% | 95% |
| 0.5 | 113% | 67% | 40% | 103% | 109% | 79% |

### Example 7—Determine Residual Starch in Liquefied Corn Mashes Treated with Thermostable Xylanase from Dictyoglomus thermophilum (Dt) and Thermotoga maritima (Tm)

Liquefaction was carried out in a metal canister using Labomat BFA-24 (Mathis, Concord, N.C.). In the canister was added 37.9 g of industrial produced ground corn to 62.0 g tap water and mixed well. The target dry solid (DS) was about 33% DS. pH was adjusted to pH 5.0 with 40% v/v sulfuric acid and dry solid was measured using moisture balance (Mettler-Toledo). Alpha-amylase and protease blend consisting 2.4 ug of alpha-amylase AA2330 plus 2.7 ug of Pfu protease were dosed into the corn slurry with or without thermostable xylanase from Dictyoglomus thermophilum (SEQ ID NO: 1) and Thermotoga maritima (SEQ ID NO: 2). Each treatment was conducted in triplicate. As control, only alpha-amylase and protease were added without addition of xylanase. The xylanase dosage was 5 μg/g dry solids. Liquefaction took place in the Labomat chamber at 91° C. for 2 hr. Once liquefaction was complete, all canisters were cooled in an ice bath to room temperature before probing for residual starch assay. Transferred 4-5 g liquefied mash samples in pre-weighed 15 mL tubes. To determine residual starch, soluble material in the mash was removed by washing with MilliQ water. The washing step was performed by adding approximately 6 mL of MilliQ water then mix by vortex the sample vigorously and centrifuge at 3500 rpm for 5 min. The supernatant was carefully discarded, and the pellet washed additional 2 times with 6 mL MilliQ water as described above. After the washing step, the insoluble solids was suspended with 6 mL of 100 mM sodium acetate buffer, pH 5.0. Residual starch of the insoluble solids was determined by adding 50 μL of concentrated raw starch hydrolyzing enzymes consisting mixture of alpha-amylase and glucoamylase (Glucoamylase BL (GBL)). Enzyme reaction was carried out at 50° C. for overnight. Upon completion of incubation, vortex the tubes thoroughly and centrifuge at 3500 rpm for 5 min. The supernatant was harvested followed by syringe filtered with 0.2 μM filter. The filtrate was diluted appropriately into HPLC vial. Glucose concentration was quantified by HPLC and the calculation converted to the amount of starch.

### Result

The post liquefaction samples treated with Tm xylanase (SEQ ID NO: 2) showed lower residual starch compare to control or Dt xylanase-treated liquefact (SEQ ID NO: 1) (Table 10). The lower residual starch showed that Tm xylanase released more starch from the ground corn compare to Dt xylanase suggesting that Tm xylanase is more active hydrolyzing corn fiber and liberated more fiber-bound starch.

TABLE 10
Mean residual starch of liquefied sample treated
with or without thermostable xylanase

| | Xylanase dose | Residual starch post liquefaction |
|---|---|---|
| Treatment | (ug/gDS) | (% starch/gDS) |
| Control | 0 | 9.2% |
| Dt xylanase | 5 | 8.6% |
| Tm xylanase | 5 | 7.6% |

### Example 8—Determine Oligosaccharides Concentration in Liquefied Corn Mashes Treated with Thermostable Xylanase from Dictyoglomus thermophilum (Dt) and Thermotoga maritima (Tm)

Liquefaction was carried out in a metal canister using Labomat BFA-24 (Mathis, Concord, N.C.). In the canister was added 38.4 g of industrial produced ground corn to 61.4 g tap water and mixed well. The target dry solid (DS) was about 33% DS. pH was adjusted to pH 5.0 with 40% v/v sulfuric acid and dry solid was measured using moisture balance (Mettler-Toledo). Alpha-amylase and protease blend consisting 1.5 ug of alpha-amylase AA2330 plus 3.0 ug of Pfu protease were dosed into the corn slurry with or without thermostable xylanase from Dictyoglomus thermophilum (SEQ ID NO: 1) and Thermotoga maritima (SEQ ID NO: 2). Each treatment was conducted in triplicate. As control, only alpha-amylase and protease were added without addition of xylanase. The xylanase dosages were 2.5, 5 and 10 μg/g dry solids. Liquefaction took place in the Labomat chamber at 91° C. for 2 hr. Once liquefaction was complete, all canisters were cooled in an ice bath to room temperature before subjected to high performance liquid chromatography (HPLC) analysis. For sample preparation for HPLC analysis, approximately 5 g of liquefied mash was transferred into 15 mL centrifuge tube and centrifuge at 3500 rpm for 10 min. After centrifugation, the supernatant was harvested followed by syringe filtered with 0.2 μM filter into HPLC vial. The HPLC was equipped with Aminex HPX-42A column (Bio-Rad), run with MilliQ water as mobile phase at flow rate of 0.5 ml/min with temperature setting at 85° C. and equipped with refractive index detection. The oligosaccharides standard of glucose, DP1; maltose, DP2; maltotriose, DP3; maltotetraose, DP4; maltopentaose, DP5 and maltohexaose, DP6 were purchased commercially and used as references to quantify the short chain oligosaccharides produced from enzymatic reaction after liquefaction.

### Result

The liquefact samples treated with Tm xylanase (SEQ ID NO: 2) showed higher concentration of DP2, DP3, DP4, DP5 and DP6, compare to control or Dt xylanase (SEQ ID NO: 1)-treated liquefact (Table 11). The total amount of oligosaccharides (DP1 to DP6) increases proportionally with increase enzyme dose of Tm xylanase. Tm xylanase was more active which generate higher amount of short-chain oligosaccharide sugars after liquefaction due to its action of releasing more fiber-bound starch which allows the alpha-amylase to synergistically hydrolyze more starch that will enable high fermentation yields as compared to Dt xylanase.

TABLE 11
Mean concentration of oligosaccharides (DP1 to DP6) of liquefied
sample treated with or without thermostable xylanase

| | Xylanase | | | | | | | Total DP1 |
|---|---|---|---|---|---|---|---|---|
| | dose | DP1 | DP2 | DP3 | DP4 | DP5 | DP6 | to DP6 |
| Treatment | (ug/gDS) | (g/L) | (g/L) | (g/L) | (g/L) | (g/L) | (g/L) | (g/L) |
| Control | 0 | 9.76 | 13.02 | 29.19 | 12.10 | 21.94 | 53.26 | 139.27 |
| Dt | 2.5 | 9.57 | 12.47 | 28.48 | 11.91 | 21.17 | 51.54 | 135.14 |
| xylanase | 5 | 9.80 | 12.59 | 28.53 | 11.98 | 21.28 | 51.59 | 135.77 |
| | 10 | 9.59 | 12.40 | 28.38 | 11.93 | 21.10 | 51.07 | 134.47 |
| Tm | 2.5 | 9.78 | 13.25 | 29.65 | 12.45 | 22.23 | 53.21 | 140.57 |
| xylanase | 5 | 9.72 | 13.54 | 30.15 | 12.48 | 23.03 | 54.65 | 143.58 |
| | 10 | 9.85 | 14.24 | 31.20 | 12.89 | 23.93 | 56.15 | 148.25 |

### Example 9—Determine Temperature Optimum and Temperature Stability of Metal Ions Resistant Thermostable Xylanase from Thermotoga maritima (Tm)

This example demonstrates how to determine the temperature optimum and DSC melting point of an enzyme (e.g., metal ion inhibition resistant xylanase of the present disclosure, alpha-amylase, protease, etc.) to determine whether the enzyme is thermostable (e.g., suitable for use in liquefying step i)). The temperature optimum and stability of metal ion resistant thermostable xylanase from Thermotoga maritima (SEQ ID NO: 2) was assayed using 10 g/L wheat arabinoxylan (P-WAXYM, Megazyme) as substrate. The substrate solution was prepared by weighing out 0.5 g of wheat arabinoxylan into a beaker then add approximately 40 mL of MilliQ water. The solution was stirred vigorously with heated at 80° C. in microwave until the substrate completely dissolved. The solution was cooled by placing the beaker in water bath while stirring. After the solution cooled to room temperature, the solution was transferred to a volumetric flask and topped up with MilliQ water to 50 mL. The substrate and buffer mixtures were prepared by mixing 250 μl substrate solution, 200 μl 250 mM sodium acetate buffer, pH 5.0 into 1.5 ml tube and vortex.

The temperature optimum assay was carried out using PCR thermal cycler pre-set to 80° C., 83° C., 86° C., 89° C., 92° C. and 95° C., respectively. In a PCR plate, 80 μl of substrate/buffer was dispensed to mix to the wells and the reaction was initiated by adding 20 μl of appropriately diluted purified Tm xylanase. Place the cap and incubate the respective temperature for 30 min on Veriti Thermal Cycler (Thermo Fisher Scientific). After 30 min reaction, the plate was immediately cooled down in ice for 3 min followed by the PAHBAH assay described below.

The temperature stability assay was carried out using PCR thermal cycler pre-set to 4° C., 86° C., 89° C., 92° C., 95° C. and 98° C., respectively. In PCR plate, 20 μl of appropriately diluted purified Tm xylanase was dispensed and then capped and pre-incubated for 30 min. After 30 min incubation, the xylanase activity was determined by adding 80 μl of substrate/buffer mix to the wells and placing the cap and incubating for 30 min on a Veriti Thermal Cycler (Thermo Fisher Scientific). After a 30 min reaction, the plate was immediately cooled down in ice for 3 min followed by the PAHBAH assay described below. In addition, the melting temperature (Td) of Tm xylanase (SEQ ID NO: 2) was determined using differential scanning calorimetry (DSC).

Xylanase generated product of xylo-oligosaccharides was determined using p-hydroxybenzoic acid hydrazide solution (PAHBAH), which detects the amount of reducing sugars released. Add 40 μL of PAHBAH solution to each plate well and incubated at 55° C. for 20 min. After 20 min, 100 μL of the respective PAHBAH reacted supernatant was transferred to a 96-well plate and measured for color developed at 405 nm using a spectrophotometer. Enzyme activity was defined as the absorbance intensity at 405 nm. The control was enzyme with substrate/buffer reaction without the metal ions present. Blanks were substrate and buffer without enzyme addition.

### Results

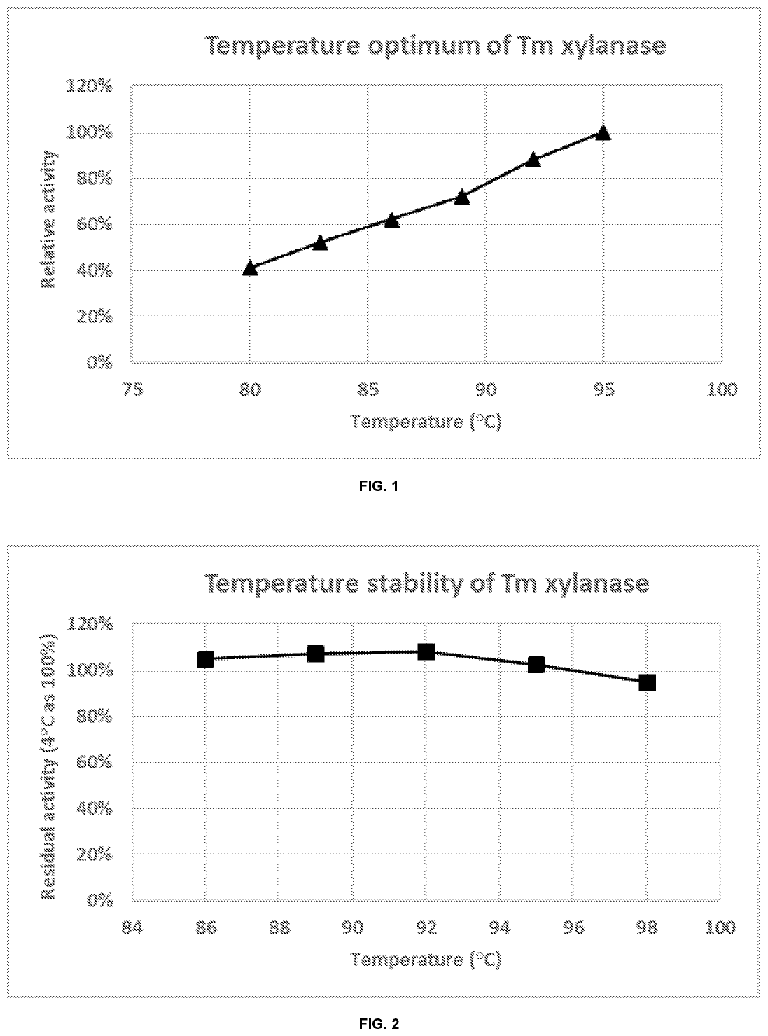

The temperature optimum of Tm xylanase (SEQ ID NO: 2) is shown in the graph of FIG. 1. The xylanase relative activity was calculated with reference to the temperature that presented the highest activity as 100%. Tm xylanase showed the highest activity at 95° C.

The temperature stability of Tm xylanase is shown in the graph of FIG. 2. The xylanase residual activity was calculated with reference to the xylanase activity pre-incubated at 4° C. as 100%. Tm xylanase is very thermostable and virtually no loss of activity at temperature up to 98° C.

The melting temperature (Td) of Tm xylanase measured on differential scanning calorimetry (DSC) has a Td value of 110° C.

1-31. (canceled)

32. A process for producing a fermentation product from a starch-containing material comprising the steps of:
i) liquefying a starch-containing material at a temperature above the initial gelatinization temperature in the presence of thermostable xylanase that is resistance to inhibition by metal ions in the liquefying starch-containing material;
ii) saccharifying using a carbohydrate-source generating enzyme; and
iii) fermenting using a fermenting organism to produce the fermentation product.

33. The process of claim 32, wherein the thermostable xylanase has a Melting Point (DSC) above 82° C.

34. The process of claim 32, wherein resistance to inhibition by metal ions in the liquefying starch-containing material is the retention of at least 60% of the relative activity of the xylanase in the presence of the average concentration of the metal ion in the liquefying starch-containing material.

35. The process of claim 32, wherein the average concentration of metal ions present in the liquefying starch-containing material ranges from 0.012 mM to 0.15 mM.

36. The process of claim 32, wherein the amount of residual starch present at the end of liquefying step i) is decreased compared to the amount of residual starch present at the end of liquefying step i) without the xylanase.

37. The process of claim 32, wherein the amount of short chain oligosaccharides present at the end of liquefying step i) is increased compared to the amount of short chain oligosaccharides at the end of liquefying step i) without the xylanase.

38. A process for decreasing the amount of residual starch present in a liquefact, comprising liquefying a starch-containing material with thermostable xylanase that is resistant to inhibition by metal ions in the liquefying starch-containing material to produce a liquefact, wherein the liquefact has a decreased amount of residual starch compared to a liquefact produced without the thermostable xylanase or when using a thermostable xylanase that is not resistant or is less resistant to inhibition by metal ions in the liquefying starch-containing material.

39. The process of claim 38, wherein the thermostable xylanase has a Melting Point (DSC) above 82° C.

40. The process of claim 38, wherein resistance to inhibition by metal ions in the liquefying starch-containing material is the retention of at least 60% of the relative activity of the xylanase in the presence of the average concentration of the metal ion in the liquefying starch-containing material.

41. The process of claim 38, wherein the average concentration of metal ions present in the liquefying starch-containing material ranges from 0.012 mM to 0.15 mM.

42. The process of claim 38, wherein the amount of residual starch present at the end of the liquefying step is decreased compared to the amount of residual starch present at the end of the liquefying step without the xylanase.

43. The process of claim 38, wherein the amount of short chain oligosaccharides present at the end of the liquefying step is increased compared to the amount of short chain oligosaccharides at the end of the liquefying step without the xylanase.

44. A process for increasing the amount of short-chain oligosaccharides present in a liquefact, comprising:
i) liquefying a starch-containing material with thermostable xylanase that is resistant to inhibition by metal ions in the liquefying starch-containing material to produce a liquefact, wherein the liquefact has an increased amount of short-chain oligosaccharides compared to a liquefact produced without the thermostable xylanase or when using a thermostable xylanase that is not resistant or is less resistant to inhibition by metal ions in the liquefying starch-containing material.

45. The process of claim 44, wherein the thermostable xylanase has a Melting Point (DSC) above 82° C.

46. The process of claim 44, wherein resistance to inhibition by metal ions in the liquefying starch-containing material is the retention of at least 60% of the relative activity of the xylanase in the presence of the average concentration of the metal ion in the liquefying starch-containing material.

47. The process of claim 44, wherein the average concentration of metal ions present in the liquefying starch-containing material ranges from 0.012 mM to 0.15 mM.

48. The process of claim 44, wherein the amount of residual starch present at the end of the liquefying step is decreased compared to the amount of residual starch present at the end of the liquefying step without the xylanase.

49. The process of claim 44, wherein the amount of short chain oligosaccharides present at the end of the liquefying step is increased compared to the amount of short chain oligosaccharides at the end of the liquefying step without the xylanase.


SEQUENCE LISTING (30 sequences)

SEQ ID NO: 1 - protein, 356 aa, Dictyoglomus thermophilum
  SIGNAL (1)..(27)
MKKPLGKIVASTALLISVAFSSSIASAKEEAKGMEIPSLKEVYKDYFTIGAAVSHLNIYHYENLLKKHFNSLTPENQMKWEVIHPKPYVYDFGPADEIVDFAMKNGMKVRGHTLVWHNQTPGWVYAGTKDEILARLKEHIKEVVGHYKGKVYAWDVVNEALSDNPNEFLRRAPWYDICGEEVIEKAFIWAHEVDPDAKLFYNDYNLEDPIKREKAYKLVKKLKDKGVPIHGIGIQGHWTLAWPTPKMLEDSIKRFAELGVEVQVTEFDISIYYDRNENNNFKVPPEDRLERQAQLYKEAFEILRKYKGIVTGVTFWGVADDYTWLYFWPVRGREDYPLLFDKNHNPKKAFWEIVKF

SEQ ID NO: 2 - protein, 328 aa, Thermotoga maritima
MQNVSLRELAEKLNIYIGFAAINNFWSLSDAEKYMEVARREFNILTPENQMKWDTIHPERDRYNFTPAEKHVEFAEENDMIVHGHTLVWHNQLPGWITGREWTKEELLNVLEDHIKTVVSHFKGRVKIWDVVNEAVSDSGTYRESVWYKTIGPEYIEKAFRWAKEADPDAILIYNDYSIEEINAKSNFVYNMIKELKEKGVPVDGIGFQMHIDYRGLNYDSFRRNLERFAKLGLQIYITEMDVRIPLSGSEEYYLKKQAEVCAKIFDICLDNPAVKAIQFWGFTDKYSWVPGFFKGYGKALLFDENYNPKPCYYAIKEVLEKKIEERK

SEQ ID NO: 3 - protein, 354 aa, Thermotoga neapolitana
MKKPLGKIVASTALLISVAFSSSIASASQDVPLRVLAEKLNIHIGFAAGNNFWSLPDAEKYMEVAKREFNILTPGNQMKWDTIHPERNRYNFEPAEKHVEFALKNDMIVHGHTLVWHNQLPGWLTGQEWSKEELLNILEDHVKTVVSHFRGRVKIWDVVNEAVSDSGTYRESIWYRTIGPEYIEKALIWAKEADPDAILIYNDYNIEEINAKSNFVYNMIKNLREKGVPIDGIGFQMHIDYRGINYESFKKNLERFAELGLQIYITEMDRGFPLGGSVGYYLKKQAEVYRRIFEICLDNPAVRAIQFWGFTDKYSWVPGFFKGYGKALIFDENYNPKPCYFAIRELMEEKLKER

SEQ ID NO: 4 - protein, 344 aa, Thermotoga naphthophila
MKILPSVLILLLGCVPVFSSQNVSLRELAEKLNIYIGFAAVNNFWSLPDAEKYMEIARREFNILTHENQMKWDTIHPERDRYNFTPAEKHVEFAEENGMIVHGHTLVWHNQLPGWLTGREWTREELLNVLEDHIKTVVSHFKGRVKIWDVVNEAVSDSGTYRESVWYKTIGPEYIEKAFRWAKEADPDAILIYNDYSIEEINAKSNFVYNMIKELKEKGVPVDGIGFQMHIDHRGLNYDSFRRNLERFAELGLQIYITEMDVRIPLSGSEEYYLKKQAEVCAKIFEICLKNPAVKAIQFWGFTDKYSWVPGFFKGYGKALLFDENYNPKPCYYAIKEVLEKKRK

SEQ ID NO: 5 - protein, 515 aa, Bacillus stearothermophilus
  mat_peptide (1)..(515)
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 6 - protein, 177 aa, Thermoascus aurantiacus
  mat_peptide (1)..(177)
TRISSCSGSRQSALTTALRNAASLANAAADAAQSGSASKFSEYFKTTSSSTRQTVAARLRAVAREASSSSSGATTYYCDDPYGYCSSNVLAYTLPSYNIIANCDIFYTYLPALTSTCHAQDQATTALHEFTHAPGVYSPGTDDLAYGYQAAMGLSSSQAVMNADTYALYANAIYLGC

SEQ ID NO: 7 - protein, 409 aa, Talaromyces leycettanus
  SIGNAL (1)..(18)
  mat_peptide (19)..(409)
MKFSNVILAASASSLVLAAPKSKTKRTSAFQWFGANESGAEFGNQNIPGTLGTDYTWPDTSTIQTLRNAGMNIFRVPFLMERLVPNQMTGSPDPTYLADLKSTVNFITGTGAYAVVDPHNYGRYYNNIITSTSDFAAFWTTVASQFASNPRVIFDTNNEYNNMDQTLVLNLNQAAINAIRAAGATSQYIFAEGNSWTGAWTWTSVNDNMKQLTDPSNKLVYEMHQYLDSDGSGTSDQCVNSTIGYDRIVSATQWLQANGKVAFLGEFAGGSNSVCEAAVTGMLDYMEQNSDVWLGAEWWAAGPWWGNYIYSMEPPSGIAYQNYLSILEPYFPGGSYSGGTGSGSGSTTTTATTTTTKVPPTSTTSSASSTGTGVAQHWGQCGGQGWTGPTTCVSPYTCQELNPYYYQCL

SEQ ID NO: 8 - DNA, 4014 bp, Thermococcus hydrothermalis
  CDS (1)..(4011)
  misc_signal (1)..(81)
  mat_peptide (82)..(4014)
atgaggcgggtggttgccctcttcattgcaattttgatgcttggaagcatcgttggagcgaacgttaagagcgttggcgcggcggagccgaagccgctcaacgtcataatagtctggcaccagcaccagccctactactacgaccctgtccaggacgtctacaccaggccctgggtcaggctccacgcggcgaacaactactggaagatggcccactacctgagccagtacccggaggttcacgccaccattgacctctcgggttcgctgatagcccagcttgccgactacatgaacggcaagaaggacacctaccagataatcaccgagaagatagccaacggggaacccctcaccgtcgacgagaagtggttcatgctccaggcaccgggagggttcttcgacaacaccatcccctggaacggtgaaccgataaccgaccccaacggcaacccgataagggacttctgggaccgctacacggagctgaagaacaagatgctcagcgcaaaggccaagtacgcaaacttcgtgactgagagccagaaggtcgctgtgacgaacgagttcacagagcaggactacatagacctagcggttctcttcaatctcgcttggattgactacaattacatcacgagcacgccggagttcaaggccctctacgacaaggttgacgagggcggctatacaagggcggacgtcaaaaccgttctcgacgcccagatctggcttctcaaccacaccttcgaggagcacgagaagataaacctcctcctcggaaacggcaacgtcgaggtcacggtcgttccctacgcccacccgataggcccgatactcaacgacttcggctgggacagcgacttcaacgaccaggtcaagaaggccgacgaactgtacaagccgtacctcggcggcggcaccgcggttccaaaaggcggatgggcggctgagagcgccctcaacgacaaaactctggagatcctcgccgagaacggctgggagtgggtcatgaccgaccagatggttctcggaaagctcggcattgagggaaccgtcgagaactaccacaagccctgggtggccgagttcaacggaaagaagatatacctcttcccaagaaatcacgatctaagtgacagagttggctttacctacagcggaatgaaccagcagcaggccgttgaggacttcgtcaacgagctcctcaagctccagaagcagaactacgatggctcgctggtttacgtggtcacgctcgacggcgagaaccccgtggagaactacccctacgacggggagctcttcctcaccgaactctacaagaagctgaccgaactccaggagcagggtctcataagaaccctcaccccgagcgagtacatccagctctacggcgacaaggccaacaagctcacacctcggatgatggagcgccttgacctcaccggagacaacgttaacgccctcctcaaggcccagagcctcggcgaactctacgacatgaccggcgttaaggaggagatgcagtggcccgagagcagctggatagacggaaccctctccacgtggataggcgagccccaggagaactacggctggtactggctctacatggccaggaaggcccttatggagaacaaggataaaatgagccaggcggactgggagaaggcctacgagtacctgctccgcgccgaggcaagcgactggttctggtggtacggaagcgaccaggacagcggccaggactacaccttcgaccgctacctgaagacctacctctacgagatgtacaagctggcaggagtcgagccgccgagctacctcttcggcaactacttcccggacggagagccctacaccacgaggggcctggtcggactcaaggacggcgagatgaagaacttctccagcatgtccccgctggcaaagggcgtgagcgtctatttcgacggcgaggggatacacttcatagtgaaagggaacctggacaggttcgaggtgagcatctgggagaaggatgagcgcgttggcaacacgttcacccgcctccaagagaagccggacgagttgagctatttcatgttcccattctcaagggacagcgttggtctcctcataaccaagcacgtcgtgtacgagaacggaaaggccgagatatacggcgccaccgactacgagaagagcgagaagcttggggaagccaccgtcaagaacacgagcgaaggaatcgaagtcgtccttccctttgactacatagaaaacccctccgacttctacttcgctgtctcgacggtcaaagatggagaccttgaggtgataagcactcctgtggagctcaagctcccgaccgaggtcaagggagtcgtcatagccgatataaccgacccagaaggcgacgaccatgggcccggaaactacacttatcccacggacaaggtcttcaagccaggtgttttcgacctcctccgcttcaggatgctcgaacagacggagagctacgtcatggagttctacttcaaggacctaggtggtaacccgtggaacggacccaacggcttcagcctccagataatcgaggtctacctcgacttcaaggacggtggaaacagttcggccattaagatgttccccgacggaccgggagccaacgtcaacctcgaccccgagcatccatgggacgttgccttcaggatagcgggctgggactacggaaacctcatcatcctgccgaacggaacggccatccagggcgagatgcagatttccgcagatccggttaagaacgccataatagtcaaggttccaaagaagtacatcgccataaacgaggactacggcctctggggagacgtcctcgtcggctcgcaggacggctacggcccggacaagtggagaacggcggcagtggatgcggagcagtggaagcttggaggtgcggacccgcaggcagtcataaacggcgtggccccgcgcgtcattgatgagctggttccgcagggctttgaaccgacccaggaggagcagctgagcagctacgatgcaaacgacatgaagctcgccactgtcaaggcgctgctactcctcaagcagggcatcgttgtgaccgacccggagggagacgaccacgggccgggaacgtacacctatccgacggacaaagttttcaagcccggtgttttcgacctcctcaagttcaaggtgaccgagggaagcgacgactggacgctggagttccacttcaaagacctcggtggaaacccgtggaacgggccgaacggcttcagcctgcagataatcgaggtatacttcgacttcaaggagggcgggaacgtctcggccattaagatgttcccggatgggcccggaagcaacgtccgtcttgatccaaatcacccatgggacctggcgcttaggatagccggctgggactacggaaacctgataattctgcccgacggaaccgcctaccaaggcgagatgcagatttccgcagatccggttaagaacgccataatagtcaaggttccaaagaagtacctgaacatatccgactacggactctacaccgccgtcatcgtgggttcccaagacgggtacggcccggacaagtggaggcccgtggccgctgaggccgagcagtggaagctcggaggcgcagacccccaggcggtcatagacaacctcgtaccaagggtcgttgatgaactcgtgccggagggcttcaagccaacgcaggaggagcagctgagcagctacgaccttgagaagaagaccctggcgacggtgctcatggtaccgctcgtcaatgggactggcggcgaggaaccaacgccgacggagagcccaacggaaacgacgacaaccacacccagcgaaacaaccaccacaacttcaacgaccaccggcccaagctcaacgaccaccagcacacccggcggaggaatctgcggcccaggcattatagcgggcctggccctgataccgctcctcctcaagaggaggaactga

SEQ ID NO: 9 - protein, 1337 aa, Thermococcus hydrothermalis
MRRVVALFIAILMLGSIVGANVKSVGAAEPKPLNVIIVWHQHQPYYYDPVQDVYTRPWVRLHAANNYWKMAHYLSQYPEVHATIDLSGSLIAQLADYMNGKKDTYQIITEKIANGEPLTVDEKWFMLQAPGGFFDNTIPWNGEPITDPNGNPIRDFWDRYTELKNKMLSAKAKYANFVTESQKVAVTNEFTEQDYIDLAVLFNLAWIDYNYITSTPEFKALYDKVDEGGYTRADVKTVLDAQIWLLNHTFEEHEKINLLLGNGNVEVTVVPYAHPIGPILNDFGWDSDFNDQVKKADELYKPYLGGGTAVPKGGWAAESALNDKTLEILAENGWEWVMTDQMVLGKLGIEGTVENYHKPWVAEFNGKKIYLFPRNHDLSDRVGFTYSGMNQQQAVEDFVNELLKLQKQNYDGSLVYVVTLDGENPVENYPYDGELFLTELYKKLTELQEQGLIRTLTPSEYIQLYGDKANKLTPRMMERLDLTGDNVNALLKAQSLGELYDMTGVKEEMQWPESSWIDGTLSTWIGEPQENYGWYWLYMARKALMENKDKMSQADWEKAYEYLLRAEASDWFWWYGSDQDSGQDYTFDRYLKTYLYEMYKLAGVEPPSYLFGNYFPDGEPYTTRGLVGLKDGEMKNFSSMSPLAKGVSVYFDGEGIHFIVKGNLDRFEVSIWEKDERVGNTFTRLQEKPDELSYFMFPFSRDSVGLLITKHVVYENGKAEIYGATDYEKSEKLGEATVKNTSEGIEVVLPFDYIENPSDFYFAVSTVKDGDLEVISTPVELKLPTEVKGVVIADITDPEGDDHGPGNYTYPTDKVFKPGVFDLLRFRMLEQTESYVMEFYFKDLGGNPWNGPNGFSLQIIEVYLDFKDGGNSSAIKMFPDGPGANVNLDPEHPWDVAFRIAGWDYGNLIILPNGTAIQGEMQISADPVKNAIIVKVPKKYIAINEDYGLWGDVLVGSQDGYGPDKWRTAAVDAEQWKLGGADPQAVINGVAPRVIDELVPQGFEPTQEEQLSSYDANDMKLATVKALLLLKQGIVVTDPEGDDHGPGTYTYPTDKVFKPGVFDLLKFKVTEGSDDWTLEFHFKDLGGNPWNGPNGFSLQIIEVYFDFKEGGNVSAIKMFPDGPGSNVRLDPNHPWDLALRIAGWDYGNLIILPDGTAYQGEMQISADPVKNAIIVKVPKKYLNISDYGLYTAVIVGSQDGYGPDKWRPVAAEAEQWKLGGADPQAVIDNLVPRVVDELVPEGFKPTQEEQLSSYDLEKKTLATVLMVPLVNGTGGEEPTPTESPTETTTTTPSETTTTTSTTTGPSSTTTSTPGGGICGPGIIAGLALIPLLLKRRN

SEQ ID NO: 10 - protein, 809 aa, Artificial Sequence
  Hybrid pullulanase of Thermoccus hydrothermalis and Thermococcus litoralis
  SIGNAL (1)..(27)
  mat_peptide (28)..(809)
MKKPLGKIVASTALLISVAFSSSIASAEEPKPLNVIIVWHQHQPYYYDPIQDIYTRPWVRLHAANNYWKMANYLSKYPDVHVAIDLSGSLIAQLADYMNGKKDTYQIVTEKIANGEPLTLEDKWFMLQAPGGFFDHTIPWNGEPVADENGNPYREQWDRYAELKDKRNNAFKKYANLPLNEQKVKITAEFTEQDYIDLAVLFNLAWIDYNYIINTPELKALYDKVDVGGYTKEDVATVLKHQMWLLNHTFEEHEKINYLLGNGNVEVTVVPYAHPIGPLLNDFGWYEDFDAHVKKAHELYKKYLGDNRVEPQGGWAAESALNDKTLEILTNNGWKWVMTDQMVLDILGIPNTVENYYKPWVAEFNGKKIYLFPRNHDLSDRVGFRYSGMNQYQAVEDFVNELLKVQKENYDGSLVYVVTLDGENPWEHYPFDGKIFLEELYKKLTELQKQGLIRTVTPSEYIQMYGDKANKLTPRMMERLDLTGDNVNALLKAQSLGELYDMTGVKEEMQWPESSWIDGTLSTWIGEPQENYGWYWLYMARKALMENKDKMSQADWEKAYEYLLRAEASDWFWWYGSDQDSGQDYTFDRYLKTYLYEMYKLAGVEPPSYLFGNYFPDGEPYTTRGLVGLKDGEMKNFSSMSPLAKGVSVYFDGEGIHFIVKGNLDRFEVSIWEKDERVGNTFTRLQEKPDELSYFMFPFSRDSVGLLITKHVVYENGKAEIYGATDYEKSEKLGEATVKNTSEGIEVVLPFDYIENPSDFYFAVSTVKDGDLEVISTPVELKLPTEVKGVVIADITDPEGDDHGPGNYT

SEQ ID NO: 11 - protein, 412 aa, Pyrococcus furiosus
  mat_peptide (1)..(412): Pyrococcus furiosus protease (Pfu)
AELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYGFEKVGYYNPTDGTWTIKVVSYSGSANYQVDVVSDGSLSQPGSS

SEQ ID NO: 12 - protein, 595 aa, Penicillium oxalicum
  mat_peptide (1)..(595): mature Penicillium oxalicum glucoamylase sequence
RPDPKGGNLTPFIHKEGERSLQGILDNLGGRGKKTPGTAAGLFIASPNTENPNYYYTWTRDSALTAKCLIDLFEDSRAKFPIDRKYLETGIRDYKSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 13 - protein, 574 aa, Trametes cingulata
MRFTLLTSLLGLALGAFAQSSAADAYVASESPIAKAGVLANIGPSGSKSNGAKAGIVIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQSYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSITVNLPASTTIEYKYIRKFNGAVTWESDPNNSITTPASGTFTQNDTWR

SEQ ID NO: 14 - protein, 583 aa, Rhizomucor pusillus
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 15 - protein, 618 aa, Talaromyces emersonii
MASLVAGALCILGLTPAAFARAPVAARATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 16 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRFSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWSAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTANFPYLVTPEQAIQREVISNGGNVFAVTDNGALSQMADVASQSSVSLVFVNADSGEGFISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 17 - protein, 459 aa, Trichoderma reesei
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 18 - protein, 250 aa, Thermoascus aurantiacus
MSFSKIIATAGVLASASLVAGHGFVQNIVIDGKKYYGGYLVNQYPYMSNPPEVIAWSTTATDLGFVDGTGYQTPDIICHRGAKPGALTAPVSPGGTVELQWTPWPDSHHGPVINYLAPCNGDCSTVDKTQLEFFKIAESGLINDDNPPGIWASDNLIAANNSWTVTIPTTIAPGNYVLRHEIIALHSAQNQDGAQNYPQCINLQVTGGGSDNPAGTLGTALYHDTDPGILINIYQKLSSYIIPGPPLYTG

SEQ ID NO: 19 - protein, 253 aa, Penicillium emersonii
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 20 - protein, 532 aa, Aspergillus fumigatus
MLASTFSYRMYKTALILAALLGSGQAQQVGTSQAEVHPSMTWQSCTAGGSCTTNNGKVVIDANWRWVHKVGDYTNCYTGNTWDTTICPDDATCASNCALEGANYESTYGVTASGNSLRLNFVTTSQQKNIGSRLYMMKDDSTYEMFKLLNQEFTFDVDVSNLPCGLNGALYFVAMDADGGMSKYPTNKAGAKYGTGYCDSQCPRDLKFINGQANVEGWQPSSNDANAGTGNHGSCCAEMDIWEANSISTAFTPHPCDTPGQVMCTGDACGGTYSSDRYGGTCDPDGCDFNSFRQGNKTFYGPGMTVDTKSKFTVVTQFITDDGTSSGTLKEIKRFYVQNGKVIPNSESTWTGVSGNSITTEYCTAQKSLFQDQNVFEKHGGLEGMGAALAQGMVLVMSLWDDHSANMLWLDSNYPTTASSTTPGVARGTCDISSGVPADVEANHPDAYVVYSNIKVGPIGSTFNSGGSNPGGGTTTTTTTQPTTTTTTAGNPGGTGVAQHYGQCGGIGWTGPTTCASPYTCQKLNDYYSQCL

SEQ ID NO: 21 - protein, 454 aa, Aspergillus fumigatus
MKHLASSIALTLLLPAVQAQQTVWGQCGGQGWSGPTSCVAGAACSTLNPYYAQCIPGATATSTTLTTTTAATTTSQTTTKPTTTGPTTSAPTVTASGNPFSGYQLYANPYYSSEVHTLAMPSLPSSLQPKASAVAEVPSFVWLDVAAKVPTMGTYLADIQAKNKAGANPPIAGIFVVYDLPDRDCAALASNGEYSIANNGVANYKAYIDAIRAQLVKYSDVHTILVIEPDSLANLVTNLNVAKCANAQSAYLECVDYALKQLNLPNVAMYLDAGHAGWLGWPANLGPAATLFAKVYTDAGSPAAVRGLATNVANYNAWSLSTCPSYTQGDPNCDEKKYINAMAPLLKEAGFDAHFIMDTSRNGVQPTKQNAWGDWCNVIGTGFGVRPSTNTGDPLQDAFVWIKPGGESDGTSNSTSPRYDAHCGYSDALQPAPEAGTWFQAYFEQLLTNANPSF

SEQ ID NO: 22 - protein, 334 aa, Penicillium aculeatum
  SIGNAL (1)..(18)
  mat_peptide (19)..(334)
MKFSNLVALAAAASSAMALPLTKKHAKRASSFEWFGSNESGAEFGSGNIPGVYGTDYIFPSTSAIQTLINNGMNIFRVTFMMERLVPNTMTGSFDAEYLSNLTSVVNYITEAGAHAVIDPHNYGRYYGSIISSTSDFQTFWKNVAGQFKSNSLVIFDTNNEYHDMDQTLVLNLNQAAINGIRAAGATSQYIFVEGNSYTGAWTWADVNDNLKNLTDPQNKIVYEMHQYLDSDGSGTSATCVSTTIGKERVTSATQWLQKNGKVGILGEFAGGVNDQCKTAITGMLSYLEDNSDVWRGAMWWAAGPWWGDYIFSLEPPSGTAYTGMWSTLKSYLA

SEQ ID NO: 23 - protein, 394 aa, Trichophaea saccata
  SIGNAL (1)..(20)
  mat_peptide (21)..(394)
MHSFFSLALAVAGLPALINAQQSAWGQCGGVGWTGATTCVSGYYCSKLNDYYSQCIPGTASTTTSAVSTTTTATSPTGSVCSGNRTKFKYFGVNESGAEFGNNVVPGTLGKDYTWPTTDSVDFFLGKGMNTFRIAFLMERLSPPAGGLTGTFDPTYLASLKNIASYITGKGGYAIIDPHNYGRYNGNIITDYTSFGTWCKNLASQFKSDSHIIFDTNNEYHDMDETLVFNLNQACINGIRAAGATSQLILIEGNSWTGAWTWISSGNAASLINLTDPNNNIAYEMHQYLDSDGSGTSPTCVSSTIGAERLAAATAWLQANNKKGFLGEIGAGSNDDCIAAVKGALCSMQEAGGVWLGALWWAAGPWWGDYYQSIEPPDGAAIARILPEALLPFL

SEQ ID NO: 24 - protein, 278 aa, Thielavia terrestris [sequence omitted]

SEQ ID NO: 25 - protein, 294 aa, Sordaria fimicola
  SIGNAL (1)..(21)
  mat_peptide (22)..(294)
MRSSTILQTGLVAVLPFAVQAASGSGKSTRYWDCCKPSCAWSGKASVNRPVLACDANNNPLNDANVKSGCDGGSAYTCANNSPWAVNDNLAYGFAATKLSGGTESSWCCACYALTFTSGPVSGKTLVVQSTSTGGDLGSNHFDLNMPGGGVGLFDGCKREFGGLPGAQYGGISSRSECDSFPAALKPGCQWRFDWFKNADNPEFTFKQVQCPSELTSRTGCKRNDDSQFPAFTPPSGGGSNPSTPTTPPSSGGGGSGCAAAMYAQCGGSGFSGCTNCPSGSTCKAINDYYHQCA

SEQ ID NO: 26 - protein, 186 aa, Thermobifida cellulosytica
ADVIGGNPYYFGGYRCSIGFSVRKGSDTGFATAGHCGETGTLTRSPEGVVAGSYFPGRDMGWVRLTGADTVTPLVNRYDGGTVTVTGSQEAVTGSSVCRSGSTTGWRCGIIQSKNQTVRYAEGTVTGLTRTTACAEAGDSGGPWLTGSQAQGVTSGGSGNCRTGGITYFQPINPLLSYFGLELVTG

SEQ ID NO: 27 - protein, 186 aa, Thermobifida fusca
AAIIGGNPYYFGNYRCSIGFSVRQGSQTGFATAGHCGSTGTRVSSPSGTVAGSYFPGRDMGWVRITSADTVTPLVNRYNGGTVTVTGSQEAATGSSVCRSGATTGWRCGTIQSKNQTVRYAEGTVTGLTRTTACAEGGDSGGPWLTGSQAQGVTSGGTGDCRSGGITFFQPINPLLSYFGLQLVTG

SEQ ID NO: 28 - protein, 186 aa, Thermobifida halotolerans
TDIIGGNPYYFDGYRCSIGFSVRRGSESGFATAGHCGEEGTETSDPEGTVAGAYFPGRDMGWVRITDADTVTPLVNRYNGENVTVAGSREAATGSSVCRSGSTTGWRCGTIRSKNQTVRYIEGTVTGLTRTTACAEGGDSGGPWLTGSQGQGVTSGGSGNCTLGGVTYFQPLNPLLSHFDLDLVTG

SEQ ID NO: 29 - protein, 325 aa, Thermococcus nautili
LVPEPTQPAQEIPWGIERVKAPDTWSITTGASNGVIEVAILDTGIDWDHPDLKDNIAWGVSTIGGEVSTDPADWYDGNGHGTHVAGTIAALNNDIGVVGVAPNVEIYAIKVLDDRGSGAYTDIAIGIEQALLGPDGILDKDGDGIIVGDPDDDAAEVISMSLGGPSDDEYLHDMIIKAYNYGVVIVAASGNEAADQPSYPAIYPEVIAVGATDSSDAVAYFSNLQPEVSAPGVDVLSTYPDDTYESLSGTSMATPHVSGVVALIQAAYYNKYGKVLPVGTFDDMNTNTVRGILHVTADDLGDPGWDIYYGYGIVRADLAVQAALG

SEQ ID NO: 30 - protein, 6 aa, Thermotoga
  MISC_FEATURE: Motif
YITEMD